(19) United States
(12) Patent Application Publication
(10) Pub. No.: US 2005/0196746 A1
(43) Pub. Date: Sep. 8, 2005

(54) High-density ion transport measurement biochip devices and methods

(76) Inventors: Jia Xu, San Diego, CA (US); Antonio Guia, San Diego, CA (US); George Walker, San Diego, CA (US); Mingxian Huang, San Diego, CA (US); Lei Wu, San Diego, CA (US); Sithiphong Khachonesin, San Diego, CA (US); Julian Yuan, Taipei (TW)

Correspondence Address: DAVID R PRESTON & ASSOCIATES APC, 12625 HIGH BLUFF DRIVE, SUITE 205, SAN DIEGO, CA, 92130, US

(21) Appl. No.: 11/033,015

(22) Filed: Jan. 10, 2005

Related U.S. Application Data
(63) Continuation-in-part of application No. 10/858,339, filed on Jun. 1, 2004.
(63) Continuation-in-part of application No. 10/760,866, filed on Jan. 20, 2004.
Application No. 10/760,866 is a continuation-in-part of application No. 10/428,565, filed on May 2, 2003, now abandoned.
(63) Continuation-in-part of application No. 10/642,014, filed on Aug. 16, 2003.
Application No. 10/642,014 is a continuation-in-part of application No. 10/351,019, filed on Jan. 23, 2003, now abandoned.
Application No. 10/642,014 is a continuation-in-part of application No. 10/104,300, filed on Mar. 22, 2002.
(60) Provisional application No. 60/474,508, filed on May 31, 2003.
(60) Provisional application No. 60/380,007, filed on May 4, 2002.
(60) Provisional application No. 60/351,849, filed on Jan. 24, 2002.
(60) Provisional application No. 60/311,327, filed on Aug. 10, 2001.
(60) Provisional application No. 60/278,308, filed on Mar. 24, 2001.
(60) Provisional application No. 60/535,461, filed on Jan. 10, 2004.
(60) Provisional application No. 60/585,822, filed on Jul. 6, 2004.

(51) Int. Cl. C12Q 1/00

(52) U.S. Cl. 435/4

(57) ABSTRACT

The present invention provides novel biochips, biochip-based devices, and device configurations that can be used for ion transport measurement. The chips, devices, and designs of the present invention are particularly suited to high-throughput assays such as compound screening assays using patch clamping techniques. The invention includes high-density biochips made by novel methods and methods of making high density biochips, and also provides novel upper chamber configurations and fluidics designs for upper chambers of ion transport measurement devices that can be used in high throughput patch clamp assays. The present invention also includes methods of using ion transport measuring chips and devices of the present invention.

This application is a continuation-in-part of U.S. patent application Ser. No. 10/858,339, filed Jun. 1, 2004 (attorney docket number ART-00107.P.5-US), which claims priority to U.S. provisional application No. 60/474,508, filed May 31, 2003 (expired), and is a continuation-in-part of U.S. patent application Ser. No. 10/760,866 (pending), filed Jan. 20, 2004, which is itself a continuation-in-part of U.S. patent application Ser. No. 10/428,565, filed May 2, 2003 (abandoned), which claims benefit of priority to U.S. patent application No. 60/380,007, filed May 4, 2002 (expired); a continuation-in-part of U.S. patent application Ser. No. 10/642,014, filed Aug. 16, 2003 (pending), which claims priority to U.S. patent application Ser. No. 10/351,019, filed Jan. 23, 2003 (abandoned), which claims priority to U.S. patent application No. 60/351,849 filed Jan. 24, 2002 (expired); and a continuation-in-part of U.S. patent application Ser. No. 10/104,300, filed Mar. 22, 2002 (pending), which claims priority to U.S. patent application No. 60/311,327 filed Aug. 10, 2001 (expired) and to U.S. patent application No. 60/278,308 filed Mar. 24, 2001 (expired). This application also claims priority to U.S. patent application No. 60/535,461 filed Jan. 10, 2004. This application also claims priority to U.S. patent application No. 60/585,822 filed Jul. 6, 2004. Each and every patent and patent application referred to in this paragraph is hereby incorporated by reference herein in its entirety.

### TECHNICAL FIELD

The present invention relates generally to the field of ion transport detection (“patch clamp”) devices, systems and methods, particularly those use of biochip technologies.

### BACKGROUND

Ion transports are channels, transporters, pore forming proteins, or other entities that are located within cellular membranes and regulate the flow of ions across the membrane. Ion transports participate in diverse processes, such as generating and timing of action potentials, synaptic transmission, secretion of hormones, contraction of muscles etc. Ion transports are popular candidates for drug discovery, and many known drugs exert their effects via modulation of ion transport functions or properties. For example, antiepileptic compounds such as phenytoin and lamotrigine which block voltage dependent sodium ion transports in the brain, anti-hypertension drugs such as nifedipine and diltiazem which block voltage dependent calcium ion transports in smooth muscle cells, and stimulators of insulin release such as glibenclamide and tolbutamine which block an ATP regulated potassium ion transport in the pancreas.

One popular method of measuring an ion transport function or property is the patch-clamp method, which was first reported by Neher, Sakmann and Steinback (Pflueger Arch. 375:219-278 (1978)). This first report of the patch clamp method relied on pressing a glass pipette containing acetylcholine (Ach) against the surface of a muscle cell membrane, where discrete jumps in electrical current were attributable to the opening and closing of Ach-activated ion transports.

The method was refined by fire polishing the glass pipettes and applying gentle suction to the interior of the pipette when contact was made with the surface of the cell. Seals of very high resistance (between about 1 and about 100 giga ohms) could be obtained. This advancement allowed the patch clamp method to be suitable over voltage ranges which ion transport studies can routinely be made.

A variety of patch clamp methods have been developed, such as whole cell, vesicle, outside-out and inside-out patches (Liem et al., Neurosurgery 36:382-392 (1995)). Additional methods include whole cell patch clamp recordings, pressure patch clamp methods, cell free ion transport recording, perfusion patch pipettes, concentration patch clamp methods, perforated patch clamp methods, loose patch voltage clamp methods, patch clamp recording and patch clamp methods in tissue samples such as muscle or brain (Boulton et al, Patch-Clamp Applications and Protocols, Neuromethods V. 26 (1995), Humana Press, New Jersey).

These and later methods relied upon interrogating one sample at a time using large laboratory apparatus that require a high degree of operator skill and time. Attempts have been made to automate patch clamp methods, but these have met with little success. Alternatives to patch clamp methods have been developed using fluorescent probes, such as cumarin-lipids (cu-lipids) and oxonol fluorescent dyes (Tsien et al., U.S. Pat. No. 6,107,066, issued August 2000). These methods rely upon change in polarity of membranes and the resulting motion of oxonol molecules across the membrane. This motion allows for the detection of changes in fluorescence resonance energy transfer (FRET) between cu-lipids and oxonol molecules. Unfortunately, these methods do not measure ion transport directly but measure the change of indirect parameters as a result of ionic flux. For example, the characteristics of the lipid used in the cu-lipid can alter the biological and physical characteristics of the membrane, such as fluidity and polarizability.

Thus, what is needed is a simple device and methods to measure ion transport activities directly. Preferably, these devices would utilize patch clamp detection methods because these types of methods represent a gold standard in this field of study. The present invention provides devices and methods, particularly miniaturized devices and automated methods, for the screening of chemicals or other moieties for their ability to modulate ion transport functions or properties.

### BRIEF SUMMARY OF THE INVENTION

The present invention recognizes that the determination of one or more ion transport functions or properties using direct detection methods, such as patch-clamping, whole cell recording, or single channel recording, are preferable to methods that utilize indirect detection methods, such as fluorescence-based detection systems.

The present invention provides biochips for ion transport measurement, ion transport measuring devices that comprise biochips, fluidics designs for ion transport measuring devices that comprise biochips, and methods of using the devices and biochips that allow for the direct analysis of ion transport functions or properties. The present invention provides biochips, devices, apparatuses, and methods that are particularly suited to high throughput ion transport measurement assays. The present invention provides biochips, devices, apparatuses, and methods that allow for automated detection of ion transport functions or properties. The present invention also provides methods of making biochips and devices for ion transport measurement that reduce the cost and increase the efficiency of manufacture, as well as improve the performance of the biochips and devices. These biochips and devices are particularly appropriate for automating the detection of ion transport functions or properties, particularly for screening purposes.

A first aspect of the present invention is a biochip that comprises at least one ion transport measuring means in the form of a hole through the biochip, in which at least a portion of the surface of the particle-sealing side of the biochip is hydrophobic. Preferably, a hydrophilic biochip of the present invention comprises a hydrophilic surface that surrounds the one or more ion transport measuring holes at the recording site area, and a hydrophobic surface that in turn surrounds the one or more recording site areas. In some preferred embodiments of this aspect of the invention, the ion transport measuring holes have counterbores that are microwell upper chambers, where the surface of the biochip has a hydrophobic surface exclusive of the microwells, which have a hydrophilic surface.

A second aspect of the present invention is a biochip for ion transport measurement that comprises a microchannel plate (MCP). An MCP ion transport measurement chip comprises at least two ion transport measuring holes in the form of microchannels through the MCP. Preferably, an ion transport measurement MCP also comprises microwells in the form of counterbores surrounding the two or more microchannels. Preferably, at least a portion of an ion transport measurement MCP is treated to increase the electrical sealing properties of the two or more ion transport measuring holes. One or more portions of an MCP ion transport measuring chip can optionally be coated with a hydrophobic material to prevent fluid contact between microwells or holes. The present invention includes methods of making MCP ion transport measuring chips. The present invention also comprises ion transport measuring devices that comprise at least one MCP ion transport measuring chip, and methods of using devices that comprise at least one MCP ion transport measuring chip for measuring one or more ion transport activities or properties of at least one particle.

A third aspect of the present invention is a flexible ion transport measurement biochip that comprises at least two ion transport measuring means in the form of holes through the flexible biochip. Preferably, a flexible ion transport measurement biochip comprises at least one flexible material that can be at least partially coated with silicon dioxide or glass. At least a portion flexible ion transport measuring chip of the present invention can be treated to improve its electrical sealing properties. One or more portions of the surface of a flexible ion transport measuring chip can be coated with a hydrophobic material to prevent fluid contact between ion transport measuring holes. A flexible ion transport measuring chip of the present invention can optionally be stored on, supported by, or dispensed from, one or more spools or one or more guide structures. The present invention includes devices that include flexible ion transport measurement biochips and methods of using such devices for measuring one or more ion transport activities or properties.

A fourth aspect of the present invention is a method of making an ion transport measurement device using theta tubing segments. The method comprises drilling holes in the septa of two or more theta tubing segments and then fusing the two or more theta tubing segments to produce an ion transport measurement device that comprises at least two ion transport measurement means in the form of holes through the septa of the tubing segments. In one embodiment, the theta tubing segments are fused one on top of another. In an alternative embodiment, the theta tubing segments are fused side-by-side. In the theta tubing-based devices of the present invention, upper and lower compartments of the theta tubing segments provide upper and lower chambers for ion transport measurement assays. In preferred embodiments, inflow and outflow conduits are attached to the openings on either side of the upper and lower compartments of each theta segment. At least a portion of the surface of the septum of a theta segment of an ion transport measurement device of the present invention can be treated to improve the electrical sealing properties of the ion transport measuring hole of the septum. The present invention includes theta tubing-based ion transport measuring devices made using the methods of the present invention, and methods of using such devices to measure one or more ion transport activities or properties of one or more particles.

A fifth aspect of the present invention is an ion transport measurement device that comprises a biochip that comprises two or more ion transport measuring holes, a common upper chamber, and an upper chamber separator unit, wherein the upper chamber separator unit comprises separator segments that can form the walls of individual upper chamber compartments when the unit is lowered onto the top of the biochip to divide the common upper chamber into at least two upper chamber compartments, each of which is in register with one of the two or more ion transport measuring holes. The present invention includes methods of using ion transport measurement devices having a biochip and multiple upper chambers formed by an upper chamber separator unit to measure one or more ion transport activities or properties.

A sixth aspect of the present invention is an ion transport measurement device that comprises a biochip that comprises two or more ion transport measuring holes, and at least two upper chambers, where the walls of the chamber are fabricated onto the biochip and comprise wax, a polymer, or an O-ring. The present invention comprises devices for ion transport measurement that comprises a chip having built-on upper chambers, and methods of using these devices for ion transport measurement assays.

A seventh aspect of the invention is an ion transport measurement device comprising a biochip that comprises at least one ion transport measuring hole and at least one flow-through upper chamber that comprises at least one inlet and at least one outlet. In some embodiments, a device comprises two or more flow-through upper chambers, and the chip comprises two or more ion transport measuring holes, each of which accesses a single flow-through upper chamber. In other embodiments, the chip comprises two or more ion transport measuring holes that access a single flow-through upper chamber. A flow-through chamber can be arranged as a channel having an inlet at one end, two or more ion transport measuring holes positioned in a linear fashion along the course of the channel, and an outlet at the opposite end. In some preferred embodiments, a flow-through chamber can have a top that is transparent, such that particles (such as cells) in the chamber can be viewed microscopically. A device of the present invention having one or more flow-through upper chambers can further comprise one or more lower chambers. The present invention also includes the use of ion transport measuring devices having flow-through upper channels to measure one or more ion transport activities or properties.

An eighth aspect of the present invention is an ion transport measuring device comprising a biochip that comprises at least two ion transport measuring holes and at least one flow-through upper chamber positioned above the biochip, and further comprising at least two delivery conduits that can be positioned over the ion transport hole recording sites to deliver liquid samples, suspensions, or solutions to ion transport recording sites. In preferred embodiments, the upper chamber comprises microwells which encompass the ion transport recording sites. In preferred embodiments, the upper surface of the chip comprises flow retarding structures that restrict the flow of fluids to the recording sites. In some preferred embodiments of this aspect, the conduits comprise multichannel pipets that can deliver solutions to a recording site. In some preferred embodiments of this aspect, the conduits comprise fluidic pipes that can deliver solutions to a recording site. In some preferred embodiments, the fluid conduits comprise funnel structures, in which solutions are delivered from the tip of the funnel and the funnel structure acts as a flow retarding structure. In some preferred embodiments, at least a portion of biochip, excluding the recording sites, is hydrophobic. Devices of the present invention having a flow-through upper chamber with overhead fluid delivery to multiple recording sites can also include two or more lower chambers, where each of the lower chambers is in register with one of the ion transport measuring holes of the chip. The present invention also includes methods of using ion transport measurement devices having an upper chamber fluid conduit delivery system to measure one or more ion transport activities or properties.

A ninth aspect of the present invention is an ion transport measuring device that comprises 1) a biochip that comprises two or more ion transport measuring holes, 2) at least two upper chambers positioned above the chip where the two or more upper chambers are in register with the two or more ion transport measuring holes, 3) a lower chamber, 4) at least two microwells on the lower surface of the chip, in which each of the two or more microwells is positioned around one of the two or more ion transport measuring holes and connected to the lower chamber, and 5) a compound delivery plate, in which the compound delivery plate has two or more drug delivery sites that can align with the two or more microwells of the chip. The compound delivery plate can be reversibly positioned under the biochip such that the two or more compound delivery sites are in close proximity to the two or more microwells to deliver compounds to the microwells. In one embodiment, the compound delivery sites are loci where compounds can be spotted or printed. In another embodiment, the compound delivery sites are apertures in the compound delivery plate through which drugs can be pumped, injected, or extruded using sonic piezo elements. Preferably, the two or more upper chambers of the device are connected to pneumatic devices that can be used to seal cells in the microwells to the underside of the chip. In some preferred embodiments, at least a portion of the lower surface of the chip that is outside of the microwells is hydrophobic. The present invention includes methods of using ion transport measurement devices having compound delivery plates for compound delivery to one or more recording sites of a chip to measure one or more ion transport activities or properties.

In a related aspect, the present invention comprises a device that comprises: 1) a biochip that comprises two or more ion transport measuring holes, 2) at least two lower chambers positioned below the chip where the two or more lower chambers are in register with the two or more ion transport measuring holes, 3) an upper chamber, 4) at least two microwells on the upper surface of the chip, in which each of the two or more microwells is positioned around one of the two or more ion transport measuring holes and connected to the upper chamber, and 5) a compound delivery plate, in which the compound delivery plate has two or more drug delivery sites that can align with the two or more microwells of the chip. The compound delivery plate can be reversibly positioned over the biochip such that the two or more compound delivery sites are in close proximity to the two or more microwells to deliver compounds to the microwells. In one embodiment, the compound delivery sites are loci where compounds can be spotted or printed. In another embodiment, the compound delivery sites are apertures in the compound delivery plate through which drugs can be pumped, pipeted, or injected. Preferably, the two or more lower chambers of the device are connected to pneumatic devices that can be used to seal cells in the microwells to the chip. In some preferred embodiments, at least a portion of the upper surface of the chip that is outside of the microwells is hydrophobic. The present invention includes methods of using ion transport measurement devices having compound delivery plates for compound delivery to one or more recording sites of a chip to measure one or more ion transport activities or properties.

An eleventh aspect of the present invention is a method of shipping ion transport devices that comprise biochips and at least one upper chamber or at least one lower chamber, in which at least one upper chamber or at least one lower chamber of the device is filled with a measuring solution and then packaged and shipped.

An twelfth aspect of the present invention comprises a method of performing excised patch ion transport measurement comprising: sealing a cell to an ion transport measuring hole in a chamber of an ion transport measuring device; adding magnetic beads to the chamber comprising the cell, in which the magnetic beads have been coated with at least one specific binding member that binds one or more molecules present on the surface of the cell; incubating the coated magnetic beads with the cell in the chamber; applying a magnet to the cell to remove the magnetic beads and a portion of the cell from the ion transport measuring site to leave an excised patch at the ion transport measuring site; and measuring ion transport activity of the excised patch.

### BRIEF DESCRIPTION OF THE DRAWINGS

FIG. 1 depicts a cross-sectional view of a portion of a chip having a hydrophobic coating and microwells.

FIG. 2 depicts one embodiment of an ion transport measuring chip made from an MCP. A) Top view. B) cross-sectional view showing etched microwells and through-holes.

FIG. 3 depicts two embodiments of a flexible chip of the present invention. A) the chip extends between two spools, with the assay area localized to the extended portion of the chip between them. B) the assay area of the chip corresponds to a portion chip that curves over a spool, which can comprise or engage chambers for ion transport assays.

FIG. 4 depicts preferred embodiments of the present invention: ion channel measuring devices that comprises theta tubing. A) a segment of theta tubing shown “face on” in which the opening for laser access (used in making the hole) is shown. B) an ion transport measuring device comprising multiple theta units arranged vertically. The upper and lower chambers of each unit have separate conduit attachments for ES (extracellular solution) and IS (intracellular solution), respectively. C) an ion transport measuring device comprising multiple theta units arranged side-by-side. Although conduits connecting with only one of the units are shown, each of the upper and lower chambers of each unit have separate conduit attachments for ES and IS, respectively.

FIG. 5 is a cross-sectional view of one embodiment of the present invention comprising a chip having a flow-through upper chamber.

FIG. 6 is a cross-sectional depiction of one embodiment of an ion transport measuring device comprising flow-through upper and lower chambers and a reservoir.

FIG. 7 depicts one embodiment of an ion transport measuring device having an upper chamber separator unit that lowers onto the chip.

FIG. 8 depicts one embodiment of a chip of the present invention in which wax forms the upper chambers. A) top view. B) cross sectional view.

FIG. 9 depicts a cross-sectional view of one embodiment of a chip of the present invention in which O-rings form the upper chambers.

FIG. 10 depicts one embodiment of an ion transport measuring device having a single flow-through upper chamber in the form of a channel that accesses multiple ion transport measuring holes of a chip.

FIG. 11 depicts one embodiment of an ion transport measuring device in which compound is delivered by fluidic pipes at ion transport measuring sites.

FIG. 12 depicts a device that has nozzle structures that interface with a fluid delivery system.

FIG. 13 depicts one embodiment of an ion transport measuring device in which compound is delivered by fluid dispensing tips at ion transport measuring sites. In this embodiment, an electrode traverses the surface of the chip. A hydrophobic layer coats the electrode, except in the immediate vicinity of microwells. A) cells have been added to an upper chamber channel comprising ES. B) cells seal to ion transport measuring holes within microwells that access the channel. C) compound drops are dispensed directly over the ion transport measuring sites. D) compound solution floods the microwell, but does not flow into neighboring microwells.

FIG. 14 depicts an ion transport chip having flow-retarding structures.

FIG. 15 depicts one embodiment of an ion transport measuring device having a compound delivery plate that delivers compounds to ion transport measuring sites.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Terms of orientation such as “up” and “down”, “top” and “bottom”, “upper” or “lower” and the like refer to orientation of parts during use of a device. Where a term is provided in the singular, the inventors also contemplate the plural of that term. Where there are discrepancies in terms and definitions used in references that are incorporated by reference, the terms used in this application shall have the definitions given herein. As employed throughout the disclosure, the following terms, unless otherwise indicated, shall be understood to have the following meanings:

“Ion transport measurement” is the process of detecting and measuring the movement of charge and/or conducting ions across a membrane (such as a biological membrane), or from the inside to the outside of a particle or vice versa. In most applications, particles will be cells, organelles, vesicles, biological membrane fragments, artificial membranes, bilayers or micelles. In general, ion transport measurement involves achieving a high resistance electrical seal of a membrane or particle with a surface that has an aperture, and positioning electrodes on either side of the membrane or particle to measure the current and/or voltage across the portion of the membrane sealed over the aperture, or “clamping” voltage across the membrane and measuring current applied to an electrode to maintain that voltage. However, ion transport measurement does not require that a particle or membrane be sealed to an aperture if other means can provide electrode contact on both sides of a membrane. For example, a particle can be impaled with a needle electrode and a second electrode can be provided in contact with the solution outside the particle to complete a circuit for ion transport measurement. Several techniques collectively known as “patch clamping” can be included as “ion transport measurement”.

An “ion transport measuring means” refers to a structure that can be used to measure at least one ion transport function, property, or a change in ion channel function, property in response to various chemical, biochemical or electrical stimuli. Typically, an ion transport measuring means is a structure with an opening that a particle can seal against, but this need not be the case. For example, needles as well as holes, apertures, capillaries, and other detection structures of the present invention can be used as ion transport measuring means. An ion transport measuring means is preferably positioned on or within a biochip or a chamber. Where an ion transport measuring means refers to a hole or aperture, the use of the terms “ion transport measuring means” “hole” or “aperture” are also meant to encompass the perimeter of the hole or aperture that is in fact a part of the chip or substrate (or coating) surface (or surface of another structure, for example, a channel) and can also include the surfaces that surround the interior space of the hole that is also the chip or substrate (or coating) material or material of another structure that comprises the hole or aperture.

A “hole” is an aperture that extends through a chip. Descriptions of holes found herein are also meant to encompass the perimeter of the hole that is in fact a part of the chip or substrate (or coating) surface, and can also include the surfaces that surround the interior space of the hole that is also the chip or substrate (or coating) material. Thus, in the present invention, where particles are described as being positioned on, at, near, against, or in a hole, or adhering or fixed to a hole, it is intended to mean that a particle contacts the entire perimeter of a hole, such that at least a portion of the surface of the particle lies across the opening of the hole, or in some cases, descends to some degree into the opening of the whole, contacting the surfaces that surround the interior space of the hole.

A “patch clamp detection structure” refers to a structure that is on or within a biochip or a chamber that is capable of measuring at least one ion transport function or property via patch clamp methods.

A “chip” is a solid substrate on which one or more processes such as physical, chemical, biochemical, biological or biophysical processes can be carried out. Such processes can be assays, including biochemical, cellular, and chemical assays; ion transport or ion channel function or activity determinations, separations, including separations mediated by electrical, magnetic, physical, and chemical (including biochemical) forces or interactions; chemical reactions, enzymatic reactions, and binding interactions, including captures. The micro structures or micro-scale structures such as, channels and wells, electrode elements, electromagnetic elements, may be incorporated into or fabricated on the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips of the present invention can vary considerably, for example, from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include wells fabricated on the surfaces.

A “biochip” is a chip that is useful for a biochemical, biological or biophysical process. In this regard, a biochip is preferably biocompatible.

A “recording site”, “ion transport measurement recording site”, or “ion transport recording site” is the area is the area immediately surrounding an ion transport measuring means (such as a hole). The area can include the bound particle and solution surrounding the bound particle. In devices that comprise microwells, the microwell defines the upper chamber recording site area.

A “microwell” in a device of the present invention is a well in a chip that has a small volumetric capacity. Preferably, the volumetric capacity of a microwell is less than about 200 microliters, and more preferably less than about 50 microliters. In devices of the present invention, a microwell surrounds an ion transport measuring hole in a chip and is prererably drilled or etched into the chip. Preferred microwells are ion transport measuring hole counterbores. Microwells preferably contain particles that are sealed to the ion transport measurement holes of a chip during use of a device that comprises microwells. Microwells can be in the upper or lower surface of a chip.

A “chamber” is a structure that comprises or engages a chip and that is capable of containing a fluid sample. The chamber may have various dimensions and its volume may vary between 0.001 microliter and 50 milliliter. In devices of the present invention, an “upper chamber” is a chamber that is above a biochip, such as a biochip that comprises one or more ion transport measuring means. In the devices of the present invention, a chip that comprises one or more ion transport measuring means can separate one or more upper chambers from one or more lower chambers. During use of a device, an upper chamber can contain measuring solutions and particles or membranes. An upper chamber can optionally comprise one or more electrodes. In devices of the present invention, a “lower chamber” is a chamber that is below a biochip. During use of a device, a lower chamber can contain measuring solutions and particles or membranes. A lower chamber can optionally comprise one or more electrodes.

A “lower chamber piece” is a part of a device for ion transport measurement that forms at least a portion of one or more lower chambers of the device. A lower chamber portion piece preferably comprises at least a portion of one or more walls of one or more lower chambers, and can optionally comprise at least a portion of a bottom surface of one or more lower chambers, and can optionally comprise one or more conduits that lead to one or more lower chambers, or one or more electrodes.

A “lower chamber base piece” is a part of a device for ion transport measurement that forms the bottom surface of one or more lower chambers of the device. A lower chamber base piece can also optionally comprise one or more walls of one or more lower chambers, one or more conduits that lead to one or more lower chambers, or one or more electrodes.

As used herein, a “platform” is a surface on which a device of the present invention can be positioned. A platform can comprises the bottom surface of one or more lower chambers of a device.

An “upper chamber piece” is a part of a device for ion transport measurement that forms at least a portion of one or more upper chambers of the device. An upper chamber piece can comprise one or more walls of one or more upper chambers, and can optionally comprise one or more conduits that lead to an upper chamber, and one or more electrodes.

An “upper chamber portion piece” is a part of a device for ion transport measurement that forms a portion of one or more upper chambers of the device. An upper chamber portion piece can comprise at least a portion of one or more walls of one or more upper chambers, and can optionally comprise one or more conduits that lead to an upper chamber, or one or more electrodes.

A “well” is a depression in a substrate or other structure. For example, in devices of the present invention, upper chambers can be wells formed in an upper chamber piece. The upper opening of a well can be of any shape and can be of an irregular conformation. The walls of a well can extend upward from the lower surface of a well at any angle or in any way. The walls can be of any shape and can be of an irregular conformation, that is, they may extend upward in a sigmoidal or otherwise curved or multi-angled fashion.

A “well hole” is a hole in the bottom of a well. A well hole can be a well-within-a well, having its own well shape with an opening at the bottom.

A “well hole piece” is a part of a device for ion transport measurement that comprises one or more well holes of the wells of the device.

When wells or chambers (including fluidic channel chambers) are “in register with” ion transport measuring means of a chip, there is a one-to-one correspondence of each of the referenced wells or chambers to each of the referenced ion transport measuring means, and an ion transport measuring means is positioned so that it is exposed to the interior of the well or chamber it is in register with, such that ion transport measurement can be performed using the chamber as a compartment for measuring current or voltage through or across the ion transport measuring means.

A “port” is an opening in a wall or housing of a chamber through which a fluid sample or solution can enter or exit the chamber. A port can be of any dimensions, but preferably is of a shape and size that allows a sample or solution to be dispensed into a chamber by means of a pipette, syringe, or conduit, or other means of dispensing a sample.

A “conduit” is a means for fluid to be transported from one area to another area of a device, apparatus, or system of the present invention or to another structure, such as a dispensation or detection device. In some aspects, a conduit can engage a port in the housing or wall of a chamber. In some aspects, a part of a device, such as, for example, an upper chamber piece or a lower chamber piece can comprise conduits in the form of tunnels that pass through the upper chamber piece and connect, for example, one area or compartment with another area or compartment. A conduit can be drilled or molded into a chip, chamber, housing, or chamber piece, or a conduit can comprise any material that permits the passage of a fluid through it, and can be attached to any part of a device. In one preferred aspect of the present invention, a conduit extends through at least a portion of a device, such as a wall of a chamber, or an upper chamber piece or lower chamber piece, and connects the interior space of a chamber with the outside of a chamber, where it can optionally connect to another conduit, such as tubing. Some preferred conduits can be tubing, such as, for example, rubber, teflon, or tygon tubing. A conduit can be of any dimensions, but preferably ranges from 10 microns to 5 millimeters in internal diameter.

A “device for ion transport measurement” or an “ion transport measuring device” is a device that comprises at least one chip that comprises one or more ion transport measuring means, at least a portion of at least one upper chamber, and, preferably, at least a portion of at least one lower chamber. A device for ion transport measurement preferably comprises one or more electrodes, and can optionally comprise conduits, particle positioning means, or application-specific integrated circuits (ASICs).

A “cartridge for ion transport measurement” comprises an upper chamber piece and at least one biochip comprising one or more ion transport measuring means attached to the upper chamber piece, such that the one or more ion transport measuring means are in register with the upper chambers of the upper chamber piece.

An “ion transport measuring unit” is a portion of a device that comprises at least a portion of a chip having an ion transport measuring means and an upper chamber, where the ion transport measuring means connects the upper chamber with a portion of a lower chamber.

A “measuring solution” is an aqueous solution containing electrolytes, with pH, osmolarity, and other physical-chemical traits that are compatible with conducting function of the ion transports to be measured.

An “intracellular solution” is a measuring solution used in the upper or lower chamber that is compatible with the electrolyte composition and physical-chemical traits of the intracellular content of a living cell.

An “extracellular solution” is a measuring solution used in the upper or lower chamber that is compatible with the electrolyte composition and physical-chemical traits of the extracellular content of a living cell.

To be “in electrical contact with” means one component is able to receive and conduct electrical signals (voltage, current, or change of voltage or current) from another component.

An “ion transport” can be any protein or non-protein moiety that modulates, regulates or allows transfer of ions across a membrane, such as a biological membrane or an artificial membrane. Ion transport include but are not limited to ion channels, proteins allowing transport of ions by active transport, proteins allowing transport of ions by passive transport, toxins such as from insects, viral proteins or the like. Viral proteins, such as the M2 protein of influenza virus can form an ion channel on cell surfaces.

A “particle” refers to an organic or inorganic particulate that is suspendable in a solution and can be manipulated by a particle positioning means. A particle can include a cell, such as a prokaryotic or eukaryotic cell, or can be a cell fragment, such as a vesicle or a microsome that can be made using methods known in the art. A particle can also include artificial membrane preparations that can be made using methods known in the art. Preferred artificial membrane preparations are lipid bilayers, but that need not be the case. A particle in the present invention can also be a lipid film, such as a black-lipid film (see, Houslay and Stanley, Dynamics of Biological Membranes, Influence on Synthesis, Structure and Function, John Wiley & Sons, New York (1982)). In the case of a lipid film, a lipid film can be provided over a hole, such as a hole or capillary of the present invention using methods known in the art (see, Houslay and Stanley, Dynamics of Biological Membranes, Influence on Synthesis, Structure and Function, John Wiley & Sons, New York (1982)). A particle preferably includes or is suspected of including at least one ion transport or an ion transport of interest. Particles that do not include an ion transport or an ion transport of interest can be made to include such ion transport using methods known in the art, such as by fusion of particles or insertion of ion transports into such particles such as by detergents, detergent removal, detergent dilution, sonication or detergent catalyzed incorporation (see, Houslay and Stanley, Dynamics of Biological Membranes, Influence on Synthesis, Structure and Function, John Wiley & Sons, New York (1982)). A microparticle, such as a bead, such as a latex bead or magnetic bead, can be attached to a particle, such that the particle can be manipulated by a particle positioning means.

A “cell” refers to a viable or non-viable prokaryotic or eukaryotic cell. A eukaryotic cell can be any eukaryotic cell from any source, such as obtained from a subject, human or non-human, fetal or non-fetal, child or adult, such as from a tissue or fluid, including blood, which are obtainable through appropriate sample collection methods, such as biopsy, blood collection or otherwise. Eukaryotic cells can be provided as is in a sample or can be cell lines that are cultivated in vitro. Differences in cell types also include cellular origin, distinct surface markers, sizes, morphologies and other physical and biological properties.

A “cell fragment” refers to a portion of a cell, such as cell organelles, including but not limited to nuclei, endoplasmic reticulum, mitochondria or golgi apparatus. Cell fragments can include vesicles, such as inside out or outside out vesicles or mixtures thereof. Preparations that include cell fragments can be made using methods known in the art.

A “population of cells” refers to a sample that includes more than one cell or more than one type of cell. For example, a sample of blood from a subject is a population of white cells and red cells. A population of cells can also include a sample including a plurality of substantially homogeneous cells, such as obtained through cell culture methods for a continuous cell lines.

A “population of cell fragments” refers to a sample that includes more than one cell fragment or more than one type of cell fragments. For example, a population of cell fragments can include mitochondria, nuclei, microsomes and portions of golgi apparatus that can be formed upon cell lysis.

A “microparticle” is a structure of any shape and of any composition that is manipulatable by desired physical force(s). The microparticles used in the methods could have a dimension from about 0.01 micron to about ten centimeters. Preferably, the microparticles used in the methods have a dimension from about 0.1 micron to about several hundred microns. Such particles or microparticles can be comprised of any suitable material, such as glass or ceramics, and/or one or more polymers, such as, for example, nylon, polytetrafluoroethylene (TEFLON™), polystyrene, polyacrylamide, sepaharose, agarose, cellulose, cellulose derivatives, or dextran, and/or can comprise metals. Examples of microparticles include, but are not limited to, plastic particles, ceramic particles, carbon particles, polystyrene microbeads, glass beads, magnetic beads, hollow glass spheres, metal particles, particles of complex compositions, microfabricated free-standing microstructures, etc. The examples of microfabricated free-standing microstructures may include those described in “Design of asynchronous dielectric micromotors” by Hagedorn et al., in Journal of Electrostatics, Volume: 33, Pages 159-185 (1994). Particles of complex compositions refer to the particles that comprise or consists of multiple compositional elements, for example, a metallic sphere covered with a thin layer of non-conducting polymer film.

“A preparation of microparticles” is a composition that comprises microparticles of one or more types and can optionally include at least one other compound, molecule, structure, solution, reagent, particle, or chemical entity. For example, a preparation of microparticles can be a suspension of microparticles in a buffer, and can optionally include specific binding members, enzymes, inert particles, surfactants, ligands, detergents, etc.

“Coupled” means bound. For example, a moiety can be coupled to a microparticle by specific or nonspecific binding. As disclosed herein, the binding can be covalent or noncovalent, reversible or irreversible.

“Micro-scale structures” are structures integral to or attached on a chip, wafer, or chamber that have characteristic dimensions of scale for use in microfluidic applications ranging from about 0.1 micron to about 20 mm. Example of micro-scale structures that can be on chips of the present invention are wells, channels, scaffolds, electrodes, electromagnetic units, or microfabricated pumps or valves.

A “particle positioning means” refers to a means that is capable of manipulating the position of a particle relative to the X-Y coordinates or X-Y-Z coordinates of a biochip. Positions in the X-Y coordinates are in a plane. The Z coordinate is perpendicular to the plane. In one aspect of the present invention, the X-Y coordinates are substantially perpendicular to gravity and the Z coordinate is substantially parallel to gravity. This need not be the case, however, particularly if the biochip need not be level for operation or if a gravity free or gravity reduced environment is present. Several particle positioning means are disclosed herein, such as but not limited to dielectric structures, dielectric focusing structures, quadropole electrode structures, electrorotation structures, traveling wave dielectrophoresis structures, concentric electrode structures, spiral electrode structures, circular electrode structures, square electrode structures, particle switch structures, electromagnetic structures, DC electric field induced fluid motion structure, acoustic structures, negative pressure structures and the like.

A “dielectric focusing structure” refers to a structure that is on or within a biochip or a chamber that is capable of modulating the position of a particle in the X-Y or X-Y-Z coordinates of a biochip using dielectric forces or dielectrophoretic forces.

A “horizontal positioning means” refers to a particle positioning means that can position a particle in the X-Y coordinates of a biochip or chamber wherein the Z coordinate is substantially defined by gravity.

A “vertical positioning means” refers to a particle positioning means that can position a particle in the Z coordinate of a biochip or chamber wherein the Z coordinate is substantially defined by gravity.

A “quadropole electrode structure” refers to a structure that includes four electrodes arranged around a locus such as a hole, capillary or needle on a biochip and is on or within a biochip or a chamber that is capable of modulating the position of a particle in the X-Y or X-Y-Z coordinates of a biochip using dielectrophoretic forces or dielectric forces generated by such quadropole electrode structures.

An “electrorotation structure” refers to a structure that is on or within a biochip or a chamber that is capable of producing a rotating electric field in the X-Y or X-Y-Z coordinates that can rotate a particle. Preferred electrorotation structures include a plurality of electrodes that are energized using phase offsets, such as 360/N degrees, where N represents the number of electrodes in the electroroation structure (see generally U.S. patent application Ser. No. 09/643,362 entitled “Apparatus and Method for High Throughput Electrorotation Analysis” filed Aug. 22, 2000, naming Jing Cheng et al. as inventors). A rotating electrode structure can also produce dielectrophoretic forces for positioning particles to certain locations under appropriate electric signal or excitation. For example, when N=4 and electrorotation structure corresponds to a quadropole electrode structure.

A “traveling wave dielectrophoresis structure” refers to a structure that is on or within a biochip or a chamber that is capable of modulating the position of a particle in the X-Y or X-Y-Z coordinates of a biochip using traveling wave dielectrophoretic forces (see generally U.S. patent application Ser. No. 09/686,737 filed Oct. 10, 2000, to Xu, Wang, Cheng, Yang and Wu; and U.S. application Ser. No. 09/678,263, entitled “Apparatus for Switching and Manipulating Particles and Methods of Use Thereof” filed on Oct. 3, 2000 and naming as inventors Xiaobo Wang, Weiping Yang, Junquan Xu, Jing Cheng, and Lei Wu).

A “concentric circular electrode structure” refers to a structure having multiple concentric circular electrodes that are on or within a biochip or a chamber that is capable of modulating the position of a particle in the X-Y or X-Y-Z coordinates of a biochip using dielectrophoretic forces.

A “spiral electrode structure” refers to a structure having multiple parallel spiral electrode elements that is on or within a biochip or a chamber that is capable of modulating the position of a particle in the X-Y or X-Y-Z coordinates of a biochip using dielectric forces.

A “square spiral electrode structure” refers to a structure having multiple parallel square spiral electrode elements that are on or within a biochip or a chamber that is capable of modulating the position of a particle in the X-Y or X-Y-Z coordinates of a biochip using dielectrophoretic or traveling wave dielectrophoretic forces.

A “particle switch structure” refers to a structure that is on or within a biochip or a chamber that is capable of transporting particles and switching the motion direction of a particle or particles in the X-Y or X-Y-Z coordinates of a biochip. The particle switch structure can modulate the direction that a particle takes based on the physical properties of the particle or at the will of a programmer or operator (see, generally U.S. application Ser. No. 09/678,263, entitled “Apparatus for Switching and Manipulating Particles and Methods of Use Thereof” filed on Oct. 3, 2000 and naming as inventors Xiaobo Wang, Weiping Yang, Junquan Xu, Jing Cheng, and Lei Wu.

An “electromagnetic structure” refers to a structure that is on or within a biochip or a chamber that is capable of modulating the position of a particle in the X-Y or X-Y-Z coordinates of a biochip using electromagnetic forces. See generally U.S. patent application Ser. No. 09/685,410 filed Oct. 10, 2000, to Wu, Wang, Cheng, Yang, Zhou, Liu and Xu and WO 00/54882 published Sep. 21, 2000 to Zhou, Liu, Chen, Chen, Wang, Liu, Tan and Xu.

A “DC electric field induced fluid motion structure” refers to a structure that is on or within a biochip or a chamber that is capable of modulating the position of a particle in the X-Y or X-Y-Z coordinates of a biochip using DC electric field that produces a fluidic motion.

An “electroosomosis structure” refers to a structure that is on or within a biochip or a chamber that is capable of modulating the position of a particle in the X-Y or X-Y-Z coordinates of a biochip using electroosmotic forces. Preferably, an electroosmosis structure can modulate the positioning of a particle such as a cell or fragment thereof with an ion transport measuring means such that the particle's seal (or the particle's sealing resistance) with such ion transport measuring means is increased.

An “acoustic structure” refers to a structure that is on or within a biochip or a chamber that is capable of modulating the position of a particle in the X-Y or X-Y-Z coordinates of a biochip using acoustic forces. In one aspect of the present invention, the acoustic forces are transmitted directly or indirectly through an aqueous solution to modulate the positioning of a particle. Preferably, an acoustic structure can modulate the positioning of a particle such as a cell or fragment thereof with an ion transport measuring means such that the particle's seal with such ion transport measuring means is increased.

A “negative pressure structure” refers to a structure that is on or within a biochip or a chamber that is capable of modulating the position of a particle in the X-Y or X-Y-Z coordinates of a biochip using negative pressure forces, such as those generated through the use of pumps or the like. Preferably, a negative pressure structure can modulate the positioning of a particle such as a cell or fragment thereof with an ion transport measuring means such that the particle's seal with such ion transport measuring means is increased.

“Dielectrophoresis” is the movement of polarized particles in electrical fields of nonuniform strength. There are generally two types of dielectrophoresis, positive dielectrophoresis and negative dielectrophoresis. In positive dielectrophoresis, particles are moved by dielectrophoretic forces toward the strong field regions. In negative dielectrophoresis, particles are moved by dielectrophoretic forces toward weak field regions. Whether moieties exhibit positive or negative dielectrophoresis depends on whether particles are more or less polarizable than the surrounding medium.

A “dielectrophoretic force” is the force that acts on a polarizable particle in an AC electrical field of non-uniform strength. The dielectrophoretic force \vec{F}_{DEP} acting on a particle of radius r subjected to a non-uniform electrical field can be given, under the dipole approximation, by:
\vec{F}_{DEP}=2πεₘr³χ_{DEP}∇Eᵣₘₛ²
where Eᵣₘₛ is the RMS value of the field strength, the symbol ∇ is the symbol for gradient-operation, εₘ is the dielectric permittivity of the medium, and χ_{DEP} is the particle polarization factor, given by:

$${χ}_{DEP} = Re ( \frac{{ɛ}_{p}^{*} - {ɛ}_{m}^{*}}{{ɛ}_{p}^{*} + 2 {ɛ}_{m}^{*}} ) ,$$

“Re” refers to the real part of the “complex number”. The symbol εₓ*=εₓ−jσₓ/2πf is the complex permittivity (of the particle x=p, and the medium x=m) and j=\sqrt{−1}. The parameters εₚ and σₚ are the effective permittivity and conductivity of the particle, respectively. These parameters may be frequency dependent. For example, a typical biological cell will have frequency dependent, effective conductivity and permittivity, at least, because of cytoplasm membrane polarization. Particles such as biological cells having different dielectric properties (as defined by permittivity and conductivity) will experience different dielectrophoretic forces. The dielectrophoretic force in the above equation refers to the simple dipole approximation results. However, the dielectrophoretic force utilized in this application generally refers to the force generated by non-uniform electric fields and is not limited by the dipole simplification. The above equation for the dielectrophoretic force can also be written as
\vec{F}_{DEP}=2πεₘr³χ_{DEP}V²∇p(x,y,z)
where p(x,y,z) is the square-field distribution for a unit-voltage excitation (Voltage V=1 V) on the electrodes, V is the applied voltage.

“Traveling-wave dielectrophoretic (TW-DEP) force” refers to the force that is generated on particles or molecules due to a traveling-wave electric field. An ideal traveling-wave field is characterized by the distribution of the phase values of AC electric field components, being a linear function of the position of the particle. In this case the traveling wave dielectrophoretic force \vec{F}_{TW-DEP} on a particle of radius r subjected to a traveling wave electrical field E=E cos(2π(ft−z/λ₀)\vec{a}ₓ (i.e., a x-direction field is traveling along the z-direction) is given, again, under the dipole approximation, by

$${{F}^{->}}_{TW - DEP} = - \frac{4 {π}^{2} {ɛ}_{m}}{{λ}_{0}} {r}^{3} {ζ}_{TW - DEP} {E}^{2} \cdot {{a}^{->}}_{z}$$

where E is the magnitude of the field strength, εₘ is the dielectric permittivity of the medium. ζ_{TV-DEP} is the particle polarization factor, given by

$${ζ}_{TW - DEP} = Im ( \frac{{ɛ}_{p}^{*} - {ɛ}_{m}^{*}}{{ɛ}_{p}^{*} + 2 {ɛ}_{m}^{*}} ) ,$$

“Im” refers to the imaginary part of the “complex number”. The symbol εₓ*=εₓ−jσₓ/2πf is the complex permittivity (of the particle x=p, and the medium x=m). The parameters εₚ and σₚ are the effective permittivity and conductivity of the particle, respectively. These parameters may be frequency dependent.

A traveling wave electric field can be established by applying appropriate AC signals to the microelectrodes appropriately arranged on a chip. For generating a traveling-wave-electric field, it is necessary to apply at least three types of electrical signals each having a different phase value. An example to produce a traveling wave electric field is to use four phase-quardrature signals (0, 90, 180 and 270 degrees) to energize four linear, parallel electrodes patterned on the chip surfaces. Such four electrodes may be used to form a basic, repeating unit. Depending on the applications, there may be more than two such units that are located next to each other. This will produce a traveling-electric field in the spaces above or near the electrodes. As long as electrode elements are arranged following certain spatially sequential orders, applying phase-sequenced signals will result in establishing traveling electrical fields in the region close to the electrodes.

“Electric field pattern” refers to the field distribution in space or in a region of interest. An electric field pattern is determined by many parameters, including the frequency of the field, the magnitude of the field, the magnitude distribution of the field, and the distribution of the phase values of the field components, the geometry of the electrode structures that produce the electric field, and the frequency and/or magnitude modulation of the field.

“Dielectric properties” of a particle are properties that determine, at least in part, the response of a particle to an electric field. The dielectric properties of a particle include the effective electric conductivity of a particle and the effective electric permittivity of a particle. For a particle of homogeneous composition, for example, a polystyrene bead, the effective conductivity and effective permittivity are independent of the frequency of the electric field at least for a wide frequency range (e.g. between 1 Hz to 100 MHz). Particles that have a homogeneous bulk composition may have net surface charges. When such charged particles are suspended in a medium, electrical double layers may form at the particle/medium interfaces. Externally applied electric field may interact with the electrical double layers, causing changes in the effective conductivity and effective permittivity of the particles. The interactions between the applied field and the electrical double layers are generally frequency dependent. Thus, the effective conductivity and effective permittivity of such particles may be frequency dependent. For moieties of nonhomogeneous composition, for example, a cell, the effective conductivity and effective permittivity are values that take into account the effective conductivities and effective permittivities of both the membrane and internal portion of the cell, and can vary with the frequency of the electric field. In addition, the dielectrophoretic force experience by a particle in an electric field is dependent on its size; therefore, the overall size of particle is herein considered to be a dielectric property of a particle. Properties of a particle that contribute to its dielectric properties include but are not limited to the net charge on a particle; the composition of a particle (including the distribution of chemical groups or moieties on, within, or throughout a particle); size of a particle; surface configuration of a particle; surface charge of a particle; and the conformation of a particle. Particles can be of any appropriate shape, such as geometric or non-geometric shapes. For example, particles can be spheres, non-spherical, rough, smooth, have sharp edges, be square, oblong or the like.

“Magnetic forces” refer to the forces acting on a particle due to the application of a magnetic field. In general, particles have to be magnetic or paramagnetic when sufficient magnetic forces are needed to manipulate particles. For a typical magnetic particle made of super-paramagnetic material, when the particle is subjected to a magnetic field \vec{B}, a magnetic dipole \vec{μ} is induced in the particle

$${μ}^{->} = {V}_{p} ( {χ}_{p} - {χ}_{m} ) \frac{{B}^{->}}{{μ}_{m}} , = {V}_{p} ( {χ}_{p} - {χ}_{m} ) {{H}^{->}}_{m}$$

where Vₚ is the particle volume, χₚ and χₘ are the volume susceptibility of the particle and its surrounding medium, μₘ is the magnetic permeability of medium, \vec{H}ₘ is the magnetic field strength. The magnetic force \vec{F}_{magnetic} acting on the particle is determined, under the dipole approximation, by the magnetic dipole moment and the magnetic field gradient:
\vec{F}_{magnetic}=−0.5Vₚ(χₚ−χₘ)\vec{H}ₘ•∇\vec{B}ₘ,
where the symbols “•” and “∇” refer to dot-product and gradient operations, respectively. Whether there is magnetic force acting on a particle depends on the difference in the volume susceptibility between the particle and its surrounding medium. Typically, particles are suspended in a liquid, non-magnetic medium (the volume susceptibility is close to zero) thus it is necessary to utilize magnetic particles (its volume susceptibility is much larger than zero). The particle velocity v_{particle} under the balance between magnetic force and viscous drag is given by:

$${v}_{particle} = \frac{{{F}^{->}}_{magnetic}}{6 π {rη}_{m}}$$

where r is the particle radius and ηₘ is the viscosity of the surrounding medium.

As used herein, “manipulation” refers to moving or processing of the particles, which results in one-, two- or three-dimensional movement of the particle, in a chip format, whether within a single chip or between or among multiple chips. Non-limiting examples of the manipulations include transportation, focusing, enrichment, concentration, aggregation, trapping, repulsion, levitation, separation, isolation or linear or other directed motion of the particles. For effective manipulation, the binding partner and the physical force used in the method should be compatible. For example, binding partner such as microparticles that can be bound with particles, having magnetic properties are preferably used with magnetic force. Similarly, binding partners having certain dielectric properties, for example, plastic particles, polystyrene microbeads, are preferably used with dielectrophoretic force.

A “sample” is any sample from which particles are to be separated or analyzed. A sample can be from any source, such as an organism, group of organisms from the same or different species, from the environment, such as from a body of water or from the soil, or from a food source or an industrial source. A sample can be an unprocessed or a processed sample. A sample can be a gas, a liquid, or a semi-solid, and can be a solution or a suspension. A sample can be an extract, for example a liquid extract of a soil or food sample, an extract of a throat or genital swab, or an extract of a fecal sample. Samples are can include cells or a population of cells. The population of cells can be a mixture of different cells or a population of the same cell or cell type, such as a clonal population of cells. Cells can be derived from a biological sample from a subject, such as a fluid, tissue or organ sample. In the case of tissues or organs, cells in tissues or organs can be isolated or separated from the structure of the tissue or organ using known methods, such as teasing, rinsing, washing, passing through a grating and treatment with proteases. Samples of any tissue or organ can be used, including mesodermally derived, endodermally derived or ectodermally derived cells. Particularly preferred types of cells are from the heart and blood. Cells include but are not limited to suspensions of cells, cultured cell lines, recombinant cells, infected cells, eukaryotic cells, prokaryotic cells, infected with a virus, having a phenotype inherited or acquired, cells having a pathological status including a specific pathological status or complexed with biological or non-biological entities.

“Separation” is a process in which one or more components of a sample is spatially separated from one or more other components of a sample or a process to spatially redistribute particles within a sample such as a mixture of particles, such as a mixture of cells. A separation can be performed such that one or more particles is translocated to one or more areas of a separation apparatus and at least some of the remaining components are translocated away from the area or areas where the one or more particles are translocated to and/or retained in, or in which one or more particles is retained in one or more areas and at least some or the remaining components are removed from the area or areas. Alternatively, one or more components of a sample can be translocated to and/or retained in one or more areas and one or more particles can be removed from the area or areas. It is also possible to cause one or more particles to be translocated to one or more areas and one or more moieties of interest or one or more components of a sample to be translocated to one or more other areas. Separations can be achieved through the use of physical, chemical, electrical, or magnetic forces. Examples of forces that can be used in separations include but are not limited to gravity, mass flow, dielectrophoretic forces, traveling-wave dielectrophoretic forces, and electromagnetic forces.

“Capture” is a type of separation in which one or more particles is retained in one or more areas of a chip. In the methods of the present application, a capture can be performed when physical forces such as dielectrophoretic forces or electromagnetic forces are acted on the particle and direct the particle to one or more areas of a chip.

An “assay” is a test performed on a sample or a component of a sample. An assay can test for the presence of a component, the amount or concentration of a component, the composition of a component, the activity of a component, the electrical properties of an ion transport protein, etc. Assays that can be performed in conjunction with the compositions and methods of the present invention include, but not limited to, biochemical assays, binding assays, cellular assays, genetic assays, ion transport assay, gene expression assays and protein expression assays.

A “binding assay” is an assay that tests for the presence or the concentration of an entity by detecting binding of the entity to a specific binding member, or an assay that tests the ability of an entity to bind another entity, or tests the binding affinity of one entity for another entity. An entity can be an organic or inorganic molecule, a molecular complex that comprises, organic, inorganic, or a combination of organic and inorganic compounds, an organelle, a virus, or a cell. Binding assays can use detectable labels or signal generating systems that give rise to detectable signals in the presence of the bound entity. Standard binding assays include those that rely on nucleic acid hybridization to detect specific nucleic acid sequences, those that rely on antibody binding to entities, and those that rely on ligands binding to receptors.

A “biochemical assay” is an assay that tests for the composition of or the presence, concentration, or activity of one or more components of a sample.

A “cellular assay” is an assay that tests for or with a cellular process, such as, but not limited to, a metabolic activity, a catabolic activity, an ion transport function or property, an intracellular signaling activity, a receptor-linked signaling activity, a transcriptional activity, a translational activity, or a secretory activity.

An “ion transport assay” is an assay useful for determining ion transport functions or properties and testing for the abilities and properties of chemical entities to alter ion transport functions. Preferred ion transport assays include electrophysiology-based methods which include, but are not limited to patch clamp recording, whole cell recording, perforated patch or whole cell recording, vesicle recording, outside out and inside out recording, single channel recording, artificial membrane channel recording, voltage gated ion transport recording, ligand gated ion transport recording, stretch activated (fluid flow or osmotic) ion transport recording, and recordings on energy requiring ion transporters (such as ATP), non energy requiring transporters, and channels formed by toxins such a scorpion toxins, viruses, and the like. See, generally Neher and Sakman, Scientific American 266:44-51 (1992); Sakmann and Heher, Ann. Rev. Physiol. 46:455-472 (1984); Cahalan and Neher, Methods in Enzymology 207:3-14 (1992); Levis and Rae, Methods in Enzymology 207:14-66 (1992); Armstrong and Gilly, Methods in Enzymology 207:100-122 (1992); Heinmann and Conti, Methods in Enzymology 207:131-148 (1992); Bean, Methods in Enzymology 207:181-193 (1992); Leim et al., Neurosurgery 36:382-392 (1995); Lester, Ann. Rev. Physiol 53:477-496 (1991); Hamill and McBride, Ann. Rev. Physiol 59:621-631 (1997); Bustamante and Varranda, Brazilian Journal 31:333-354 (1998); Martinez-Pardon and Ferrus, Current Topics in Developmental Biol. 36:303-312 (1998); Herness, Physiology and Behavior 69:17-27 (2000); Aston-Jones and Siggins, www.acnp.org/GA/GN40100005/CH005.html (Feb. 8, 2001); U.S. Pat. No. 6,117,291; U.S. Pat. No. 6,107,066; U.S. Pat. No. 5,840,041 and U.S. Pat. No. 5,661,035; Boulton et al., Patch-Clamp Applications and Protocols, Neuromethods V. 26 (1995), Humana Press, New Jersey; Ashcroft, Ion Channels and Disease, Cannelopathies, Academic Press, San Diego (2000); Sakmann and Neher, Single Channel Recording, second edition, Plenuim Press, New York (1995) and Soria and Cena, Ion Channel Pharmacology, Oxford University Press, New York (1998), each of which is incorporated by reference herein in their entirety.

An “electrical seal” refers to a high-resistance engagement between a particle such as a cell or cell membrane and an ion transport measuring means, such as a hole, capillary or needle of a chip or device of the present invention. Preferred resistance of such an electrical seal is between about 1 mega ohm and about 100 giga ohms, but that need not be the case. Generally, a large resistance results in decreased noise in the recording signals. For specific types of ion channels (with different magnitude of recording current) appropriate electric sealing in terms of mega ohms or giga ohms can be used.

An “acid” includes acid and acidic compounds and solutions that have a pH of less than 7 under conditions of use.

A “base” includes base and basic compounds and solutions that have a pH of greater than 7 under conditions of use.

“More electronegative” means having a higher density of negative charge. In the methods of the present invention, a chip or ion transport measuring means that is more electronegative has a higher density of negative surface charge.

An “electrolyte bridge” is a liquid (such as a solution) or a solid (such as an agar salt bridge) conductive connection with at least one component of the electrolyte bridge being an electrolyte so that the bridge can pass current with no or low resistance.

A “ligand gated ion transport” refers to ion transporters such as ligand gated ion channels, including extracellular ligand gated ion channels and intracellular ligand gated ion channels, whose activity or function is activated or modulated by the binding of a ligand. The activity or function of ligand gated ion transports can be detected by measuring voltage or current in response to ligands or test chemicals. Examples include but are not limited to GABA_{A}, strychnine-sensitive glycine, nicotinic acetylcholine (Ach), ionotropic glutamate (iGlu), and 5-hydroxytryptamine₃ (5-HT₃) receptors.

A “voltage gated ion transport” refers to ion transporters such as voltage gated ion channels whose activity or function is activated or modulated by voltage. The activity or function of voltage gated ion transports can be detected by measuring voltage or current in response to different commanding currents or voltages respectively. Examples include but are not limited to voltage dependent Na⁺ channels.

“Perforated” patch clamp refers to the use of perforation agents such as but not limited to nystatin or amphotericin B to form pores or perforations that are preferably ion-conducting, which allows for the measurement of current, including whole cell current.

An “electrode” is a structure of highly electrically conductive material. A highly conductive material is a material with conductivity greater than that of surrounding structures or materials. Suitable highly electrically conductive materials include metals, such as gold, chromium, platinum, aluminum, and the like, and can also include nonmetals, such as carbon, conductive liquids and conductive polymers. An electrode can be any shape, such as rectangular, circular, castellated, etc. Electrodes can also comprise doped semi-conductors, where a semi-conducting material is mixed with small amounts of other “impurity” materials. For example, phosphorous-doped silicon may be used as conductive materials for forming electrodes.

A “channel” is a structure with a lower surface and at least two walls that extend upward from the lower surface of the channel, and in which the length of two opposite walls is greater than the distance between the two opposite walls. A channel therefore allows for flow of a fluid along its internal length. A channel can be covered (a “tunnel”) or open.

“Continuous flow” means that fluid is pumped or injected into a chamber of the present invention continuously during the separation process. This allows for components of a sample that are not selectively retained on a chip to be flushed out of the chamber during the separation process.

“Binding partner” refers to any substances that both bind to the moieties with desired affinity or specificity and are manipulatable with the desired physical force(s). Non-limiting examples of the binding partners include cells, cellular organelles, viruses, particles, microparticles or an aggregate or complex thereof, or an aggregate or complex of molecules.

A “specific binding member” is one of two different molecules having an area on the surface or in a cavity that specifically binds to and is thereby defined as complementary with a particular spatial and polar organization of the other molecule. A specific binding member can be a member of an immunological pair such as antigen-antibody, can be biotin-avidin or biotin streptavidin, ligand-receptor, nucleic acid duplexes, IgG-protein A, DNA-DNA, DNA-RNA, RNA-RNA, and the like.

A “nucleic acid molecule” is a polynucleotide. A nucleic acid molecule can be DNA, RNA, or a combination of both. A nucleic acid molecule can also include sugars other than ribose and deoxyribose incorporated into the backbone, and thus can be other than DNA or RNA. A nucleic acid can comprise nucleobases that are naturally occurring or that do not occur in nature, such as xanthine, derivatives of nucleobases, such as 2-aminoadenine, and the like. A nucleic acid molecule of the present invention can have linkages other than phosphodiester linkages. A nucleic acid molecule of the present invention can be a peptide nucleic acid molecule, in which nucleobases are linked to a peptide backbone. A nucleic acid molecule can be of any length, and can be single-stranded, double-stranded, or triple-stranded, or any combination thereof. The above described nucleic acid molecules can be made by a biological process or chemical synthesis or a combination thereof.

A “detectable label” is a compound or molecule that can be detected, or that can generate readout, such as fluorescence, radioactivity, color, chemiluminescence or other readouts known in the art or later developed. Such labels can be, but are not limited to, photometric, colorimetric, radioactive or morphological such as changes of cell morphology that are detectable, such as by optical methods. The readouts can be based on fluorescence, such as by fluorescent labels, such as but not limited to, Cy-3, Cy-5, phycoerythrin, phycocyanin, allophycocyanin, FITC, rhodamine, or lanthanides; and by fluorescent proteins such as, but not limited to, green fluorescent protein (GFP). The readout can be based on enzymatic activity, such as, but not limited to, the activity of beta-galactosidase, beta-lactamase, horseradish peroxidase, alkaline phosphatase, or luciferase. The readout can be based on radioisotopes (such as ³³P, ³H, ¹⁴C, ³⁵S, ¹²⁵I, ³²P or ¹³¹I). A label optionally can be a base with modified mass, such as, for example, pyrimidines modified at the C5 position or purines modified at the N7 position. Mass modifying groups can be, for examples, halogen, ether or polyether, alkyl, ester or polyester, or of the general type XR, wherein X is a linking group and R is a mass-modifying group. One of skill in the art will recognize that there are numerous possibilities for mass-modifications useful in modifying nucleic acid molecules and oligonucleotides, including those described in Oligonucleotides and Analogues: A Practical Approach, Eckstein, ed. (1991) and in PCT/US94/00193.

A “signal producing system” may have one or more components, at least one component usually being a labeled binding member. The signal producing system includes all of the reagents required to produce or enhance a measurable signal including signal producing means capable of interacting with a label to produce a signal. The signal producing system provides a signal detectable by external means, often by measurement of a change in the wavelength of light absorption or emission. A signal producing system can include a chromophoric substrate and enzyme, where chromophoric substrates are enzymatically converted to dyes, which absorb light in the ultraviolet or visible region, phosphors or fluorescers. However, a signal producing system can also provide a detectable signal that can be based on radioactivity or other detectable signals.

The signal producing system can include at least one catalyst, usually at least one enzyme, and can include at least one substrate, and may include two or more catalysts and a plurality of substrates, and may include a combination of enzymes, where the substrate of one enzyme is the product of the other enzyme. The operation of the signal producing system is to produce a product that provides a detectable signal at the predetermined site, related to the presence of label at the predetermined site.

In order to have a detectable signal, it may be desirable to provide means for amplifying the signal produced by the presence of the label at the predetermined site. Therefore, it will usually be preferable for the label to be a catalyst or luminescent compound or radioisotope, most preferably a catalyst. Preferably, catalysts are enzymes and coenzymes that can produce a multiplicity of signal generating molecules from a single label. An enzyme or coenzyme can be employed which provides the desired amplification by producing a product, which absorbs light, for example, a dye, or emits light upon irradiation, for example, a fluorescer. Alternatively, the catalytic reaction can lead to direct light emission, for example, chemiluminescence. A large number of enzymes and coenzymes for providing such products are indicated in U.S. Pat. No. 4,275,149 and U.S. Pat. No. 4,318,980, which disclosures are incorporated herein by reference. A wide variety of non-enzymatic catalysts that may be employed are found in U.S. Pat. No. 4,160,645, issued Jul. 10, 1979, the appropriate portions of which are incorporated herein by reference.

The product of the enzyme reaction will usually be a dye or fluorescer. A large number of illustrative fluorescers are indicated in U.S. Pat. No. 4,275,149, which is incorporated herein by reference.

Other technical terms used herein have their ordinary meaning in the art that they are used, as exemplified by a variety of technical dictionaries.

Introduction

The present invention recognizes that using direct detection methods to determine an ion transport functions or properties, such as patch-clamp techniques, is preferable to using indirect detection methods, such as fluorescence-based detection systems. The present invention provides biochips and methods of use that allow for the direct detection of one or more ion transport functions or properties using chips and devices that can allow for automated detection of one or more ion transport functions or properties. These biochips and methods of use thereof are particularly appropriate for automating the detection of ion transport functions or properties, particularly for screening purposes.

As a non-limiting introduction to the breath of the present invention, the present invention includes several general and useful aspects, including:
1) a biochip that comprises at least one ion transport measuring means in the form of a hole through the biochip, in which at least a portion of the surface of the biochip is hydrophobic.
2) a biochip for ion transport measurement that comprises a microchannel plate (MCP).
3) a flexible ion transport measurement biochip.
4) methods of making an ion transport measurement device using theta tubing segments.
5) an ion transport measurement device that comprises a biochip that comprises multiple ion transport measuring holes, a common upper chamber, and an upper chamber separator unit.
6) an ion transport measurement device that comprises a biochip that comprises multiple ion transport measuring holes and multiple upper chambers, where the walls of the chambers are fabricated onto the biochip.
7) an ion transport measurement device comprising a biochip that comprises at least one ion transport measuring hole and at least one flow-through upper chamber.
8) a device comprising a biochip that comprises multiple ion transport measuring hole accessing a single flow-through upper chamber, further comprising at least two delivery conduits that can be positioned over the ion transport measuring hole recording sites to deliver solutions to the recording sites.
9) an ion transport measurement device that comprises: a compound delivery plate, in which the compound delivery plate has multiple drug delivery sites that can align with microwells on the underside of the chip to deliver compounds to ion transport recording sites.
10) an ion transport measurement device that comprises a compound delivery plate, in which the compound delivery plate has multiple drug delivery sites that can align with the two or more microwells on the upper surface of the chip to deliver compounds to ion transport recording sites.
11) methods of shipping ion transport devices that comprise biochips and at least one upper chamber or at least one lower chamber, in which the upper chamber or chambers or the lower chamber or chambers of the device are pre-filled with a measuring solution.
12) a method of performing excised patch ion transport measurement comprising:
sealing a cell to an ion transport measuring hole; adding coated magnetic beads to the chamber, removing the magnetic beads and a portion of the cell from the ion transport measuring site with a magnet to leave an excised patch at the ion transport measuring site; and measuring ion transport activity of the excised patch.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

Biochips for Ion Transport Measurement

The present invention includes chip-based devices for ion transport measurement. The ion transport measuring chips used in the present invention comprise a substrate and at least one hole through the substrate, where the hole serves as the ion transport measuring means. Preferably, the devices can be used to perform multiple ion transport assays at the same time (or in very rapid succession), and therefore preferred chips used in the methods of the present invention comprise two or more holes. For performing ion transport measurement assays, an ion transport measuring device of the present invention preferably comprises a chip, at least one upper chamber (fluid compartment) situated above the chip, and at least one lower chamber (fluid compartment) situated below the chip, in which an ion transport measuring hole through the chip provides fluid communication between a lower chamber and an upper chamber (when there is no particle sealed to the hole). The upper surface of the chip forms the bottom, or at least a portion of the bottom, of at least one upper chamber, and the lower surface of the chip forms the bottom, or at least a portion of the top, of at least one lower chamber. When a chip comprises multiple holes, upper chambers are in register with the holes when they are aligned over the chip such that each upper chamber is accessed by one hole of the chip. In the same sense, lower chambers are in register with the holes when they are aligned under the chip such that each lower chamber is accessed by one hole of the chip. The walls of upper and lower chambers can be built onto or into the chip, or can be made up of one or more separate pieces that reversibly or irreversibly engage the chip. An upper chamber may have a top or cover or may be open at the top. A lower chamber may have a bottom or may be open at the bottom. During use of an ion transport device, particles (such as cells) can be sealed to the top (or upper) surface or the bottom (or lower) surface of the chip. The entire surface of the chip (upper or lower) to which particles seal during use of the device is herein referred to as the sealing surface of the chip, regardless of whether particles seal to the particular area of the surface referred to on the “sealing surface”.

The chip can comprises any solid material such as metals, ceramics, polymers, inorganic and organic hybrid materials, plastics, silicon dioxide, or glass. The substrate can be from about 5 microns to more than 1,000 microns thick (thicker substrates may require counterbores). A substrate of from about 10 to about 200 microns in thickness is preferred. Preferably, a chip used in an ion transport measuring device is biocompatible (does not have a deleterious effect on cells) referred to herein as a “biochip”. A nonbiocompatible substrate material can be may biocompatible by coating with a suitable material.

Ion transport measuring holes can be etched, drilled, cut, punched out, milled, or bored into the substrate. In some preferred embodiments, the chip is a glass chip and the ion transport measuring holes are laser drilled. The diameter of ion transport measuring holes is preferably from about 0.2 micron to 10 microns, more preferably from about 0.5 micron to 5 microns, and most preferably from 0.5 micron to 3 microns.

A chip of the present invention used for ion transport measurement can comprise one or more microwells that encompasses an ion transport measurement hole. A microwell is a counterbore drilled or etched into the surface of a chip at the site of an ion transport measuring hole that can hold a volume of liquid (such as measurement solution) and can therefore serve as an upper or lower chamber. Drilling counterbores into a chip at the site of an ion transport measuring hole thins the chip at the site of the ion transport measuring hole, and thus reduces hole depth and hole resistance. The design and fabrication of counterbores in ion transport measuring chips is described in parent U.S. patent application Ser. No. 10/858,339, herein incorporated by reference in its entirety for all disclosure of ion transport measuring chip and chip fabrication methods. In some aspects of the present invention counterbores can be used as microwells to retain small volumes of solution such as a measuring solution or a compound solution at a recording site.

Preferably, a chip of the present invention is surface-treated to enhance its electrical sealing properties, such as by using methods described herein and in parent U.S. patent application Ser. No. 10/858,339, herein incorporated by reference in its entirety for descriptions of treatment of chips to increase electrical sealing properties.

In some preferred embodiments, an ion transport measuring chip is single-use and disposable, but this is not a requirement of the invention. In some embodiments, for example, a chamber that comprises a chip is washed or flushed out between successive uses. Depending upon the design of the device, an upper chamber piece, a lower chamber piece, or both, as well as associated electrodes (which can be part of the signal amplifier machinery or electrodes that can be attached or connected to the wells), are preferably but optionally reusable. In some embodiments of the present invention chamber electrodes can be supplied by “adaptor plates” that reversibly engage at least a portion of one or more upper chambers or one or more lower chambers. Adaptor plates can be reused by detaching the plate from a first device used in a first set of assays and attaching the plate to a second device to be used in a second set of assays. Adaptor plates can also include one or more inflow conduits, one or more outflow conduits, or one or more conduits that connects to a pneumatic device such as a pump or syringe that can be used to seal particles to ion transport measuring means of the device.

Treating Chips Comprising Ion Transport Measuring Means to Enhance the Electrical Seal of a Particle

Ion transport measuring means includes, as non-limiting examples, holes, apertures, capillaries, and needles. “Modifying an ion transport measuring means” or “Treating an ion transport measuring means” means modifying at least a portion of the surface of a chip, substrate, coating, channel, or other structure that comprises or surrounds the ion transport measuring means. The modification may refer to the surface surrounding all or a portion of the ion transport measuring means. For example, a biochip of the present invention that comprises an ion transport measuring means can be modified on one or both surfaces (e.g. upper and lower surfaces) that surround an ion transport measuring hole, and the modification may or may not extend through all or a part of the surface surrounding the portion of the hole that extends through the chip. Similarly, for capillaries, pipettes, or for channels or tube structures that comprises ion transport measuring means (such as apertures), the inner surface, outer surface, or both, of the channel, tube, capillary, or pipette can be modified, and all or a portion of the surface that surrounds the inner aperture and extends through the substrate (and optionally, coating) material can also be modified.

As used herein, “enhance the electrical seal”, “enhance the electric seal”, “enhance the electric sealing” or “enhance the electrical sealing properties (of a chip or an ion transport measuring means)” means increase the resistance of an electrical seal that can be achieved using an ion transport measuring means, increase the efficiency of obtaining a high resistance electrical seal (for example, reducing the time necessary to obtain one or more high resistance electrical seals), or increasing the probability of obtaining a high resistance electrical seal (for example, the number of high resistance seals obtained within a given time period).

Preferably, treating an ion transport measuring means to enhance the electrical sealing properties results in a change in surface properties of the ion transport measuring means. The change in surface properties can be a change in surface texture, a change in surface cleanness, a change in surface composition such as ion composition, a change in surface adhesion properties, or a change in surface electric charge on the surface of the ion transport measuring means. In some preferred aspects of the present invention, a substrate or structure that comprises an ion transport measuring means is subjected to chemical treatment (for example, treated in acid and/or base for specified lengths of time under specified conditions). For example, treatment of a glass chip comprising a hole through the chip as an ion transport measuring means with acid and/or base solutions may result in a cleaner and smoother surface in terms of surface texture for the hole. In addition, treating a surface of a biochip or fluidic channel that comprises an ion transport measuring means (such as a hole or aperture) or treating the surface of a pipette or capillary with acid and/or base may alter the surface composition, and/or modify surface hydrophobicity and/or change surface charge density and/or surface charge polarity.

Preferably, the altered surface properties improve or facilitate a high resistance electric seal or high resistance electric sealing between the surface-modified ion transport measuring means and a membranes or particle. In preferred embodiments of the present invention in which the ion transport measuring means are holes through one or more biochips, one or more biochips having ion transport measuring means with enhanced sealing properties (or, simply, a “biochip having enhanced sealing properties”) preferably has a rate of at least 50% high resistance sealing, in which a seal of 1 Giga Ohm or greater occurs at 50% of the ion transport measuring means takes place in under 2 minutes after a cell lands on an ion transport measuring hole, and preferably within 10 seconds, and more preferably, in 2 seconds or less. Preferably, for biochips with enhanced sealing properties, a 1 Giga Ohm resistance seal is maintained for at least 3 seconds.

In practice, in preferred aspects of the present invention the method comprises providing an ion transport measuring means and treating the ion transport measuring means with one or more of the following: heat, a laser, microwave radiation, high energy radiation, salts, reactive compounds, oxidizing agents (for example, peroxide, oxygen plasma), acids, or bases. Preferably, an ion transport measuring means or a structure (as nonlimiting examples, a structure can be a substrate, chip, tube, or channel, any of which can optionally comprise a coating) that comprises at least one ion transport measuring means is treated with one or more agents to alter the surface properties of the ion transport measuring means to make at least a portion of the surface of the ion transport measuring means smoother, cleaner, or more electronegative.

An ion transport measuring means can be any ion transport measuring means, including a pipette, hole, aperture, or capillary. An aperture can be any aperture, including an aperture in a channel, such as within the diameter of a channel (for example, a narrowing of a channel), in the wall of a channel, or where a channel forms a junction with another channel. (As used herein, “channel” also includes subchannels.) In some preferred aspects of the present invention, the ion transport measuring means is on a biochip, on a planar structure, but the ion transport measuring means can also be on a non-planar structure.

The ion transport measuring means or surface surrounding the ion transport measuring means modified to enhance electrical sealing can comprise any suitable material. Preferred materials include silica, glass, quartz, silicon, plastic materials, polydimethylsiloxane (PDMS), or oxygen plasma treated PDMS. In some preferred aspects of the present invention, the ion transport measuring means comprises SiOM surface groups, where M can be hydrogen or a metal, such as, for example, Na, K, Mg, Ca, etc. In such cases, the surface density of said SiOM surface groups (or oxidized SiOM groups (SiO⁻)) is preferably more than about 1%, more preferably more than about 10%, and yet more preferably more than about 30%. The SiOM group can be on a surface, for example, that comprises glass, for example quartz glass or borosilicate glass, thermally oxidized SiO₂ on silicon, deposited SiO₂, deposited glass, polydimethylsiloxane (PDMS), or oxygen plasma treated PDMS.

In preferred embodiments, the method comprises treating said ion transport measuring means with acid, base, salt solutions, oxygen plasma, or peroxide, by treating with radiation, by heating (for example, baking or fire polishing) by laser polishing said ion transport measuring means, or by performing any combinations thereof.

An acid used for treating an ion transport measuring means can be any acid, as nonlimiting examples, HCl, H₂SO₄, NaHSO₄, HSO₄, HNO₃, HF, H₃PO₄, HBr, HCOOH, or CH₃COOH can be. The acid can be of a concentration about 0.1 M or greater, and preferably is about 0.5 M or higher in concentration, and more preferably greater than about 1 M in concentration. Optimal concentrations for treating an ion transport measuring means to enhance its electrical sealing properties can be determined empirically. The ion transport measuring means can be placed in a solution of acid for any length of time, preferably for more than one minute, and more preferably for more than about five minutes. Acid treatment can be done under any non-frozen and non-boiling temperature, preferably at greater or equal than room temperature.

An ion transport measuring means, or a chip comprising an ion transport measuring means, can be treated with a base, such as a basic solution, that can comprise, as nonlimiting examples, NaOH, KOH, Ba(OH)₂, LiOH, CsOH, or Ca(OH)₂. The basic solution can be of a concentration of about 0.01 M or greater, and preferably is greater than about 0.05 M, and more preferably greater than about 0.1 M in concentration. Optimal concentrations for treating an ion transport measuring means to enhance its electrical sealing properties can be determined empirically. The ion transport measuring means can be placed in a solution of base for any length of time, preferably for more than one minute, and more preferably for more than about five minutes. Base treatment can be done under any non-frozen and non-boiling temperature, preferably at greater or equal than room temperature.

An ion transport measuring means can be treated with a salt, such as a metal salt solution, that can comprise, as nonlimiting examples, NaCl, KCl, BaCl₂, LiCl, CsCl, Na₂SO₄, NaNO₃, or CaCl, etc. The salt solution can be of a concentration of about 0.1 M or greater, and preferably is greater than about 0.5 M, and more preferably greater than about 1 M in concentration. Optimal concentrations for treating an ion transport measuring means to enhance its electrical sealing properties can be determined empirically. The ion transport measuring means can be placed in a solution of metal salt for any length of time, preferably for more than one minute, and more preferably for more than about five minutes. Salt solution treatment can be done under any non-frozen and non-boiling temperature, preferably at greater or equal than room temperature.

Where treatments such as baking, fire polishing, or laser polishing are employed, they can be used to enhance the smoothness of a glass or silica surface. Where laser polishing of a chip or substrate is used to make the surface surrounding an ion transport measuring means more smooth, it can be performed on the front side of the chip, that is, the side of the chip or substrate that will be contacted by a sample comprising particles during the use of the ion transport measuring chip or device.

Appropriate temperatures and times for baking, and conditions for fire and laser polishing to achieve the desired smoothness for improved sealing properties of ion transport measuring means can be determined empirically.

In some aspects of the present invention, it can be preferred to rinse the ion transport measuring means, such as in water (for example, deionized water) or a buffered solution after acid or base treatment, or treatment with an oxidizing agent, and, preferably but optionally, before using the ion transport measuring means to perform electrophysiological measurements on membranes, cells, or portions of cells. Where more than one type of treatment is performed on an ion transport measuring means, rinses can also be performed between treatments, for example, between treatment with an oxidizing agent and an acid, or between treatment with an acid and a base. An ion transport measuring means can be rinsed in water or an aqueous solution that has a pH of between about 3.5 and about 10.5, and more preferably between about 5 and about 9. Nonlimiting examples of suitable aqueous solutions for rinsing ion transport measuring means can include salt solutions (where salt solutions can range in concentration from the micromolar range to 5M or more), biological buffer solutions, cell media, or dilutions or combinations thereof. Rinsing can be performed for any length of time, for example from minutes to hours.

Some preferred methods of treating an ion transport measuring means to enhance its electrical sealing properties include one or more treatments that make the surface more electronegative, such as treatment with a base, treatment with electron radiation, or treatment with plasma. Not intending to be limiting to any mechanism, base treatment can make a glass surface more electronegative. This phenomenon can be tested by measuring the degree of electro-osmosis of dyes in glass capillaries that have or have not been treated with base. In such tests, increasing the electronegativity of glass ion transport measuring means correlates with enhanced electrical sealing by the base-treated ion transport measuring means. Base treatment can optionally be combined with one or more other treatments, such as, for example, treatment with heat (such as by baking or fire polishing) or laser treatment, or treatment with acid, or both. Optionally, one or more rinses in water, a buffer, or a salt solution can be performed before or after any of the treatments.

For example, after manufacture of a glass chip that comprises one or more holes as ion transport measuring means, the chip can be baked, and subsequently incubated in a base solution and then rinse in water or a dilution of PBS. In another example, after manufacture of a glass chip that comprises one or more holes as ion transport measuring means, the chip can optionally be baked, subsequently incubated in an acid solution, rinsed in water, incubated in a base solution, and finally rinsed in water or a dilution of PBS. In some preferred embodiments, the surfaces of a chip surrounding ion transport measuring means can be laser polished prior to treating the chip with acid and base. Methods and protocols for treating chips to increase their electrical sealing properties, to increase their surface hydrophilicity, and to increase their electronegativity are provided in U.S. patent application Ser. No. 10/858,339, filed Jun. 1, 2004, U.S. patent application Ser. No. 10/760,866, and U.S. patent application Ser. No. 10/760,866 all of which are hereby incorporated by reference for methods of treating chips to increase their electrical sealing properties.

The present invention includes chips such as biochips treated to enhance their electrical sealing properties, and devices comprising biochips treated to enhance their electrical sealing properties. The device can comprise at least one biochip that has been treated to enhance its electrical sealing properties, where the biochip comprises at least one hole, at least one upper chamber accessed by the at least one hole, or at least one lower chamber accessed by the at least one hole. In another embodiment, the device can comprise at least one biochip that has been treated to enhance its electrical sealing properties, where the biochip comprises at least one hole, at least one upper chamber accessed by the at least one hole and at least one lower chamber accessed by the at least one hole.

In some aspects of the present invention, it can be preferable to store an ion transport measuring means (or a chip comprising an ion transport measuring means) that has been treated to have enhanced sealing capacity in an environment having decreased carbon dioxide relative to the ambient environment. This can preserve the enhanced electrical sealing properties of the ion transport measuring means. Such an environment can be, for example, water, a salt solution (including a buffered salt solution), acetone, a vacuum, or in the presence of one or more drying agents or desiccants (for example, silica gel, CaCl₂ or NaOH) or under nitrogen or an inert gas. Where an ion transport measuring means or structure comprising an ion transport measuring means is stored in water or an aqueous solution, preferably the pH of the water or solution is greater than 4, more preferably greater than about 6, and more preferably yet greater than about 7. For example, an ion transport measuring means or a structure comprising an ion transport measuring means can be stored in a solution having a pH of approximately 8.

Glass chips that have been base treated and stored in water with neutral pH levels can maintain their enhanced sealability for as long as 10 months or longer. In addition, patch clamp chips bonded to plastic cartridges via adhesives such as UV-acrylic or UV-epoxy glues can be stored in neutral pH water for months without affecting the sealing properties.

We have also tested patch clamp biochips and cartridges that were stored in a dry environment with dessicant for 30 days. The chips were re-hydrated and tested for sealing. In one experiment, we got 6/7 seals for the dry-stored chips. Similarly, we stored mounted chips in dry environment and were able to obtain seals after a few weeks of storage.

Dehydration can, however, reduce the sealability of chemically treated chips. To improve the seal rate for dry-stored chips, NaOH, NaCl, CaCl₂ and other salt or basic solutions can be used to rejuvenate the chips out of dry storage to restore the sealability.

Hydrophilic Chip Having Hydrophobic Modifications

One aspect of the present invention is a hydrophilic biochip for ion transport measurement that comprises a substrate comprising one or more holes, one or more hydrophilic recording site areas on a surface of the substrate to which particles seal during use of the chip (the “particle-sealing side” of the chip), and at least one hydrophobic area on the same surface of the substrate, in which at least one hydrophobic area surrounds the one or more hydrophilic recording site areas; and in which the hydrophobic area can maintain an aqueous solution localized to the hydrophilic recording site area in fluid isolation.

The surface of a “hydrophilic chip” that is to be used as the particle-sealing surface is designed so that aqueous solutions such as, for example, measuring solutions used in ion transport assays, that are deposited or distributed in a hydrophilic area of the chip surface, will remain confined to hydrophilic areas of the particle-sealing surface as the solutions are repelled by hydrophobic surfaces that surround the hydrophilic areas. On a hydrophilic chip of the present invention, recording site areas on the side of the chip to be used for particle-sealing are therefore designed to be hydrophilic. Preferably, the substrate surface recording site areas are positively or negatively charged, and more preferably, the substrate surface at recording sites of a hydrophobic chip of the present invention is negatively charged to enhance the electrical sealing at the ion transport measuring hole. Negatively charged surfaces include surfaces having negative charge that is counterbalanced by noncovalently bound positive ions.

The hydrophobic surface surrounding the recording sites can extend over the entire portion of the chip, exclusive of recording site areas, or can be discontinuous. In some apsects of the present invention, the hydrophobic area of the substrate (chip) surface comprises essentially all of the surface area of said chip on its particle sealing side, excluding the one or more hydrophilic recording areas.

In preferred embodiments of a hydrophilic chip having hydrophobic areas, the chip comprises two or more holes and two or more hydrophilic recording site areas each of which is surrounded by the hydrophobic chip surface, such that an aqueous solution provided in any hydrophilic recording site area is isolated from an aqueous solution provided in any other hydrophilic recording site area. In this way, recording site areas can be maintained in fluid isolation in the absence of structural barriers.

A hydrophilic ion transport measuring biochip of the present invention that has hydrophobic surface areas can have any number of holes, from 1 to more than one thousand. In preferred embodiments, a hydrophilic ion transport measuring biochip having hydrophobic surface areas surrounding recording sites is high-density, and can be used for high throughput screening (such as, but not limited to, compound screening), and has 384 or more ion transport measuring holes. In some embodiments, a hydrophilic ion transport measuring biochip can have 1536 or more ion transport measuring holes.

Preferably, a hydrophilic recording site area of a chip of the present invention can hold a drop of aqueous liquid of a volume of from about 1 microliter to about 2 milliliters. A hydrophilic recording site surface area can preferably have a diameter of from about 25 micron to about 10 millimeters, more preferably from about 500 micron to about 2 millimeters.

A hydrophobic biochip of the present invention can comprise microwells that define the recording site area and preferably serve as upper or even lower chambers (in embodiments where cells are sealed to the bottom surface of a chip). For example, microwells that surround ion transport measuring holes be drilled or etched into a substrate of a hydrophobic chip as counterbores. The microwell surfaces of a hydrophobic chip are hydrophilic, and the microwells can retain small volumes of solutions distributed in the microwells that are repelled by surrounding hydrophobic surfaces.

A hydrophilic biochip can comprise a hydrophilic substrate that, in areas where the surface is hydrophobic, is modified to be hydrophobic or is coated with a hydrophobic material. For example, the substrate can comprise glass, silicon, silicon dioxide, quartz, or one or more hydrophilic polymers. The thickness of the hydrophilic substrate is not limiting, but can be from about 1 micron to about 2 millimeters. The substrate material can be modified by chemical or physical means to make it hydrophobic. Alternatively, the hydrophilic substrate can be coated with at least one hydrophobic plastic or polymer, such as, for example, polyethylene, polyacrylate, polypropylene, polystyrene, or polysiloxane. The thickness of the coating is also not limiting and can be as thin as one molecular layer in thickness.

A hydrophilbic biochip having one or more hydrophobic areas can be made by providing a substrate that comprises a hydrophilic material, such as, for example, glass; coating the substrate with at least one hydrophobic material (such as a hydrophobic polymer); making at least one hole through said substrate; and removing the hydrophobic substrate from the area immediately surrounding said at least one hole to define a hydrophilic recording site area. Preferably, the recording site is chemically treated, such as with a salt or base solution, to improve the electrical sealing properties of the ion transport measuring hole at the recording site. The hole can be laser drilled or etched through a substrate such as glass, for example. The hydrophobic material can be removed from recording site areas by chemical means, however, in preferred embodiments a counterbore and through-hole are laser drilled or etched into the substrate at each recording site. The drilling or etching of the counterbore removes the hydrophobic coating to produce a hydrophilic microwell at the recording site.

An alternative method of making a hydrophilic chip with hydrophobic areas is to provide a substrate that comprises a hydrophilic material; making one or more holes through said substrate; and coat at least a portion of the substrate with at least one hydrophobic material, while masking the recording site areas around the one or more holes to prevent them from receiving the hydrophobic coating.

In yet another embodiment, a chip can comprise a hydrophobic substrate material such as a polymer or plastic, and can be coated with a hydrophilic material at recording sites on the particle sealing surface of the chip. The hydrophilic chip can be made by providing a substrate that comprises a hydrophobic material; making one or more holes through the substrate; and coating the recording area surrounding the one or more holes with at least one hydrophilic material. For example, glass can be used to coat the chip surface at the one or more recording site areas. The recording site areas can preferably be chemically treated, such as with at least one salt or at least one base, to improve the electrical sealing properties of the one or more ion transport measuring holes.

A preferred embodiment of this aspect of the present invention is a hydrophilic ion transport measuring biochip having one or more hydrophobic areas that comprises ion transport measuring means in the form of holes of from about 0.2 to 10 microns in diameter that are surrounded by counterbores, where the counterbores are microwell upper chambers.

A hydrophilic/hydrophobic chip having microwell upper chambers can be made by providing a suitable substrate, such but not limited to a glass, quartz, silicon, silicon dioxide, or one or more polymers, and coating the substrate with a hydrophobic material. Suitable materials for providing a hydrophobic coating include plastics and polymers, such as, for example, polyethylene, polyacrylate, polypropylene, polystyrene, or polysiloxane. After coating the chip, two or more holes are made, such as by laser drilling into the chip. The laser drilling has the effect of melting and burning the polymer in the area surrounding the drilled hole, provided an uncoated (hydrophilic) surface in the area where a cell (or other particle) can seal. Preferably, a counterbore is also drilled into the chip, where the counterbore can serve as a microwell on the upper surface of the chip.

This design provides upper microwells (made by laser drilling) that are in liquid fluid isolation from one another, as the hydrophobic surface between wells repels aqueous liquids such as buffers and measuring solutions. The ion transport holes and areas immediately surrounding them (such as counterbore microwells) have hydrophilic surfaces that have been exposed by the laser drilling and therefore will retain buffers and solutions.

The upper microwells can optionally be connected to a common reference electrode that can traverse the chip surface. Preferably, the electrode is coated with a nonconducting (and hydrophobic) material, such as a plastic or polymer used to coat the chip surface and traverses the surfaces of the chip. The electrode can be uncoated where it contacts the microwells, so that the microwells are in electric communication without the possibility of solution exchange or mixing between wells.

One preferred embodiment of the electrode on a hydrophilic/hydrophobic chip is a metallized layer on the substrate coated by deposition, growing, condensing, or other means. The metallized layer can be removed at and near the recording sites by laser shots or masking. The hydrophobic layer is then coated on top of the metallized layer to allow for fluidic liquid separation between two adjacent recoding sites, leaving a ring of metallized layer uncovered near the recording sites to allow for electrical connection of each recording sites (in form of a hole, or a hole and a microwell) with the metallized layer which services as a reference electrode. The metallized layer can be made of any conductive material or materials including metals, non-metals, metal derivatives, or combinations thereof.

As alternatives, individual recording electrodes can also be physically or electrically connected (such as through electrolyte bridges) to each of the upper chamber microwells. In these designs, there can be individual or common lower chambers that engage the chip, and the one or more lower chambers comprise or are electrically connected to one or more reference electrodes.

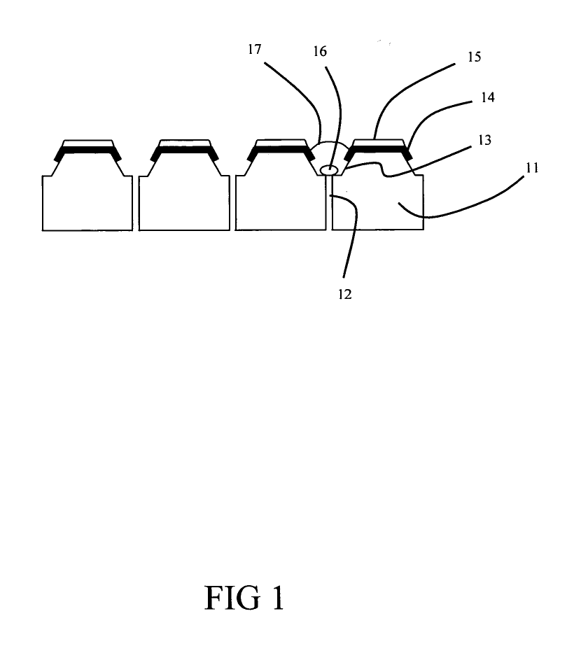

FIG. 1 shows a cross-sectional schematic view of one preferred design ion transport measuring hydrophilic/hydrophobic chip of the present invention. In this design, the chip comprises a substrate (11) such as glass or silicon dioxide that is hydrophilic, through which through holes (12) have been laser drilled to provide ion transport measuring means. Counterbores that serve as upper chamber microwells (13) have also been laser drilled into the substrate. In this design, the upper surface of the chip (the surface that serves as the sealing surface) is coated with an electrode layer (14) that contacts the microwells (13). Outside of the microwells, the electrode layer is coated with a hydrophobic material (15) that promotes fluid isolation of the microwells. The rightmost microwell in the figure is shown containing solution (17) (such as extracellular solution) that is in contact with the electrode but is excluded from the hydrophobic layer (15). A cell (16) is depicted in the well sealed to the in transport measuring hole (12).

Where the coating material is resistant to treatment chemicals, such as base and/or acid, the surface of the hole on the hydrophobic chip can be chemically treated, such as by using methods described herein, to enhance the electrical sealing properties of the chip.

The present invention also includes methods of making a hydrophilic/hydrophobic chip, and devices comprising a hydrophobic chip, where the devices can employ any feasible upper chamber, lower chamber, electrode, fluidic and pneumatic designs, such as but not limited to those described in the present application. The present invention also includes methods of using a hydrophobic ion channel measuring chip to measure ion channel activity or properties of one or more cells or particles.

Novel Ion Transport Measuring Biochip Designs

The present invention also includes novel methods of making high density and/or multiplex ion transport measuring biochips and biochips made by these methods. These devices can be used to record ion transport activity of more than one particle or cell simultaneously or in rapid sequence. In preferred aspects, the ion transport measuring biochips and biochips made by these methods are designed to be high density the ion transport measuring biochips. By “high density” is meant that the chips comprise a large number of ion transport measuring means. Typically, the ion transport measuring means are holes through the surface of the biochip, and a high density transport measuring biochip has multiple ion transport recording sites via multiple holes. In this way, multiple assays can be conducted simultaneously, or in rapid sequence, allowing for high-throughput ion transport measuring assays that can facilitate, for example, compound assays.

As used herein, “high throughput” means high quantity of independent data collected in a defined period of time. For example, 48 or more assays that can be conducted within a short time span where multiple assays are initiated simultaneously or in rapid succession, then share experimental time as parallel or multiplexed recordings, and ten completed simultaneously or independently but in parallel (less than one hour from loading of cells to completing ion channel recording, preferably, less than one half hour from loading of cells to completing ion channel recording, and more preferably, less than fifteen minutes from loading of cells to completing ion channel recording). More preferably, more than 96 high throughput ion transport measurements can be completed in less than one half hour, and more preferably yet, the high density ion transport measuring devices of the present invention are capable of performing more than 100 ion transport assays within one half hour or less. In some preferred aspects of these embodiments, high density ion transport measuring devices can perform hundreds or over one thousand assays within one half hour or less. For example, in some preferred aspects of high density ion transport measuring devices described herein, the devices can be designed to perform 384 assays or, for example, 1536 assays, within one half hour or less. For another example, 48 or more assays that can be conducted within a time span during which continuous and repetitive data sampling are performed for kinetic studies with high temporal resolution. In another example, multiple lower density assays, such as 16-assay devices, may be utilized in parallel to result in a high density assay.

While the devices herein can be described as high-throughput, the designs are not limited to high throughput uses and can be used for any number of ion transport assays, in assays that can last from seconds to several hours.

MCP-Based Chip

One aspect of the present invention is an ion transport measuring device that comprises a microchannel plate (MCP). Microchannel glass plates that comprise an array of microchannels and their fabrication are known in the art of electronics and optics for their use as electron multipliers and photomultipliers. Some aspects of their fabrication and use are described in Wiza (1979) Microchannel Plate Detectors Nuclear Instruments and Methods 162: 587-601. In brief, they can be made by providing glass fibers that have a core glass and a cladding that comprises lead glass. The fibers are arranged together side-by-side in a desirable configuration, drawn, surrounded by a glass envelope, and fused to produce a boule. The boule can be sliced (cutting perpendicular to the fiber lengths) to produce slices that are cross-sections of the boules. These slices can be finished, for example, by polishing. The cores of the glass fibers are then chemically etched away, to form the microchannel plate.

An MCP made for use as part of an ion transport measuring device can be made by fusing from 2 to over 1,000 glass fibers. An MCP ion transport measuring chip can, for example, be a high-density ion transport measuring chip that comprises 48 or more microchannels that serve as ion transport measuring holes, and preferably, 96 or more microchannels that serve as ion transport measuring holes. The core of the fibers (the portion made of etchable glass) used to make an MCP chip can be as wide as 40 microns in diameter (for chips used for ion transport assays using large cells, such as oocytes) but preferably are from 0.2 to 8 microns in diameter, and are more preferably from 0.5 to 5 microns in diameter, even more preferably from 0.5 to 3 microns in diameter, and most preferably about 2 microns in diameter. The thickness of the lead glass cladding around the core can vary depending on the desired spacing of the resulting ion transport measuring holes. The length of the fibers used in making an MCP ion transport measuring chip are not limiting, and can be of any feasible length. Preferably, after fusing the glass fibers, the boule is sliced into sections that are from about 5 microns to 5000 microns thick, most preferably from about 10 to about 50 microns thick.

The core glass fibers can be randomly arranged or configured into a pattern to make the boule.

The boule slice or wafer may be wet-etched to etch away preferentially the embedded fibers of a softer glass so as to produce micron-sized through-holes in the wafer. The softer glass fibers are more easily etched by wet etching solutions. A higher concentration, or higher reaction temperature, or combination of both, may also etch the harder glass substrate of the MCP wafer, though to a lesser degree. In this method only one side of the MCP wafer is exposed to a wet-etching compound, by floating it over a reaction chamber, or by clamping an inert gasket onto the MCP wafer such as to produce a reaction chamber with the MCP wafer at its bottom surface, under conditions that also etch the harder glass substrate. The reaction is then quenched once all of the through-holes have emerged on the opposite surface, leaving through holes that taper gradually from a larger diameter end on the etching side, to a smaller diameter emergent hole on the non-etching side. The opposing side of the MCP wafer that is not exposed to the etching compound is kept immersed in a quenching medium (such as water) that will dilute or inactivate any emerging etching compound and prevent etching on the emerging surface.

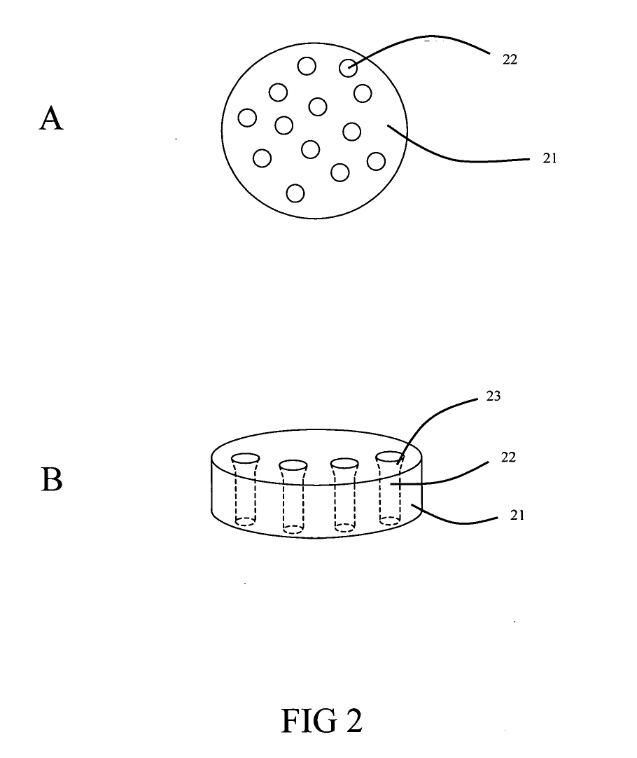

In one design, depicted in FIG. 2, the area surrounding the ion transport measuring holes on the upper side of the MCP chip (21) can be chemically wet-etched to produce microwells (23) at the upper ends of the holes (22) through the chip that can be used as upper chambers. These upper chambers can be used for measuring solution, cells or particles, and test compounds. The MCP chip can be bonded to a bottom piece that comprises one or more lower chambers. The MCP plate can also be bonded to an upper piece that comprises the ES chambers.

The surface of the MCP chip can be chemically treated, such as using methods disclosed herein, to enhance the electrical seal of a particle or membrane with the ion transport measuring means. The entire MCP chip or a portion thereof can be treated to enhance its electrical sealing properties. Preferably, at least a portion of the surface of the MCP chip to which cells or particles are to be sealed is treated with at least one salt or at least one base. One or both surfaces, or one or more portions of one or both surfaces, of the MCP chip can also be coated, or one or more portions of one or both surfaces, with one or more materials that can increase its sealing properties. In some embodiments, one or both surfaces, or one or more portions of one or both surfaces, of the MCP chip can be coated with one or more hydrophobic materials that can be used to promote fluidic isolation of individual microwells of the MCP chip. Designs in which hydrophobic surfaces are used to promote fluidic isolation of individual microwells of a chip are further described in the previous section of this application (above).

In designs in which the bottom piece forms individual lower chambers, reference electrodes can be within or electrically connected with the upper wells and recording electrodes can be within or electrically connected with the lower wells for ion transport measurement, or recording electrodes can be within or electrically connected with the upper wells and reference electrodes can be within or electrically connected with the lower wells for ion transport measurement.

In one possible design involving etched microwells on the upper surface, a common reference electrode can connect all of the upper microwells. The electrode, which can be a conductive material such as metal, can follow paths along the top surface of the MCP chip and contact measuring solution only where it contacts the interior of the microwells. The electrode can optionally be coated with a nonconductive material where it traverses the chip surface, and be exposed where it contacts the interior of the wells.

Where a device comprising an MCP chip is configured to have a common electrode that contacts multiple lower wells of the device, the same design can be used.

In designs in which the bottom piece forms a bottom chamber that contacts more than one ion transport measuring hole, the bottom chamber preferably comprises or is in electrical contact with a reference electrode, and individual upper chambers comprise individual recording electrodes. Alternatively, the bottom piece can comprise multiple lower chambers with individual recording electrodes, and the device has a common upper chamber with a reference electrode. In this embodiment, compounds can be added using compound delivery mechanisms such as, for example, fluid block delivery, chamber separators, or other mechanisms described herein.

In an alternative design for an ion transport measuring device that comprises an MCP chip, upper chambers can be constructed by attaching a manufactured piece that comprises well openings such that each well of the upper chamber piece aligns with one of the ion transport measuring holes (the microchannels of the MCP). Individual upper chambers preferably have a volume of from about 0.5 microliters to about 5 milliliters, and more preferably from about 2 microliters and about 2 milliliters, and more preferably yet between about 10 microliters and about 0.5 milliliter. The upper chamber piece can be irreversibly or reversibly attached to the MCP ion transport measuring chip using gaskets, clamps, adhesives, welding, or other means. The upper chamber piece can comprise glass, ceramics, coated metals, or (preferably) plastics or polymers. In one preferred embodiment, the upper chamber piece comprises a separate MCP. In this design, the glass fibers used to make the upper chamber piece MCP are of a wider diameter than those used to make the ion transport measuring chip MCP. The glass fibers used to make the upper chamber piece MCP also comprise a cladding of sufficient thickness to provide chamber spacing over the ion transport measuring holes of the ion transport measuring chip MCP. Conduits can connect to the wells of the upper chamber piece for the addition of solutions, cells, or compounds. Alternatively, a fluid dispensing device can interface with the upper chamber wells to dispense solutions, cells, or compounds.

A lower chamber piece can also comprise multiple chambers that connect to individual ion transport holes of the MCP chip. The lower chamber piece can be constructed by attaching a manufactured piece that comprises wells spaced such that each well of the lower chamber piece aligns with one of the ion transport measuring holes (the microchannels of the MCP). The lower chamber piece can be irreversibly or reversibly attached to the MCP ion transport measuring chip using gaskets, clamps, adhesives, welding, or other means. The upper chamber piece can comprise glass, ceramics, coated metals, or (preferably) plastics or polymers. In one embodiment, the lower chamber piece comprises a separate MCP. In this design, the glass fibers used to make the upper chamber piece MCP are of a wider diameter than those used to make the ion transport measuring chip MCP. The glass fibers used to make the lower chamber piece MCP also comprise a cladding of sufficient thickness to provide chamber spacing over the ion transport measuring holes of the ion transport measuring chip MCP. Conduits can connect to the wells of a lower chamber piece for the addition of solutions, and allowing pneumatic control. The lower well electrodes can optionally be provided by a separate adaptor plate that can reversibly engage the lower wells and can also optionally comprise connections to pneumatic devices for pressure control.

In using devices having individual upper chambers and individual lower chambers recording electrodes (or connections to recording electrodes) can be provided in or attached to upper chambers, and reference electrodes (or connections to reference electrodes) can be provided in or attached to lower chambers. In the alternative, recording electrodes (or connections to recording electrodes) can be provided in or attached to lower chambers, and reference electrodes (or connections to reference electrodes) can be provided in or attached to upper chambers.

In some preferred embodiments, however, a device that comprises an MCP ion transport measuring chip can have a single lower chamber that accesses all ion transport measuring holes of the MCP chip. In this case, the lower chamber can also comprise ceramics, coated metals, glass, plastics, or polymers, and preferably connects to conduits that connect to pressure sources and can deliver and remove fluids to and from the chamber. Pressure control may be performed from either bottom chambers or upper chambers, or both. In these embodiments, the lower chamber preferably comprises or is in electrical connection with a reference electrode during use of the device, and each upper chamber comprises or is in electrical connection with a recording electrode during use of the device.

In some other preferred embodiments, a device that comprises an MCP ion transport measuring chip can have a single upper chamber that accesses all ion transport measuring holes of the MCP chip. In this case, the upper chamber can also comprise ceramics, coated metals, glass, plastics, or polymers and it preferably comprises or is in electrical connection with a reference electrode during use of the devices. In this case each lower chamber comprises or is in electrical connection with a recording electrode during use of the device. Pressure control may be performed from either bottom chambers or upper chambers or both.

The present invention comprises ion transport measuring devices comprising an MCP chip having greater than two through holes, and at least one upper chamber. Preferably an ion transport measuring device comprising an MCP chip has multiple upper chambers that are reversibly or irreversibly attached to the MCP chip. Preferably an ion transport measuring device that comprises an MCP chip can be reversibly or irreversibly attached to at least one lower chamber. The present invention also comprises ion transport measuring devices comprising an MCP chip having multiple microchannel through holes, and an MCP chip having multiple microchannel upper chambers.

The present invention also comprises methods of using MCP chips for measuring ion transport activity and properties, as well as for other assays.

Flexible Ion Transport Measurement (ITM) Chip

Another aspect of the present invention is a method of making a flexible ion transport measuring biochip that comprises a flexible sheet of material, preferably coated with glass, comprising multiple ion transport measuring holes. The flexible sheet of material can be wound around a spool and unwound to form either a curved or an essentially flat surface for ion transport measurement. Alternatively, the flexible sheet of material can be curved to form a tube, on the surface of which ion transport measurement assays can be performed.

The method comprises: providing a substrate that comprises a sheet of flexible material; creating (for example, by laser drilling, chemical etching, micromachining, molding, etc) at least two holes in the substrate that extend through the substrate; and optionally coating the substrate with SiO₂ or glass to provide an ITM chip.

The substrate can comprise any material that can be provided as a thin sheet (for example, of within the range of between 5 and 5000 microns in thickness) and has a flexibility that allows the sheet to be curved completely around (such as to make a tube) yet is hard and rigid enough to allow manufacture of ion transport measuring holes through the substrate (that is, holes of a diameter within the range of from about 0.2 to about 8 microns in diameter, although larger diameters can be used depending on the cell type to be assayed). For example, rubber, plastics, polymers or other flexible sheet materials can be used. One such material is polyimide or Kapton. Kapton sheets of from about 5 to 5000 microns in thickness, preferably from about 10 to about 200 microns in thickness, can be laser drilled to produce through holes of within the range of from about about 0.2 to about 8 microns in diameter, preferably from about 0.5 to 5 microns in diameter, and more preferably from about 0.5 to about 3 microns in diameter. Counterbores that can be used as microwells can also optionally be drilled into the polyimide sheet, as described herein in parent U.S. application Ser. No. 10/858,339, incorporated herein in its entirety for its disclosure of counterbores and fabrication of counterbores. From 2 to over 50,000,000 holes can be drilled into a single polyimide sheet, depending on the application, which can be further rolled around a spool. For example, where a flexible biochip is to be used as a “chip roll” in which section of the flexible biochip are used to be used sequentially, the sheet can comprise a very large number of holes, a subset of which are to be used in any given assay.

Before or after laser drilling of holes in the flexible substrate, the substrate is preferably treated or coated with a material that allows for efficient and high-resistance sealing of particles such as cells to the ion transport measuring holes. The modification of the surface can be any modification that promotes high-resistance sealing of particles such as cells to the ion transport measuring holes of the chip. Preferably, the modification makes at least a portion of the surface of the flexible chip to which particles are sealed during use of the chip more hydrophilic, and more preferably, the modification makes at least a portion of the surface of the chip where particles seal negatively charged. The modification can comprise coating the surface with organic or inorganic molecules, synthetic molecules (for example, polymers) or naturally occurring ones, in liquid or non-liquid form. The coated surface can be hydrophobic or hydrophilic, charged or noncharged, and can be linked to the substrate covalently or non-covalently. The coating can be further modified to make it more hydrophilic. In one preferred embodiment, the substrate is coated with glass and the chip is treated with at least one salt or at least one base to improve its electrical sealing properties.

If the coating is a naturally rigid material, such as glass, the coating should be thin enough, or physio-chemically altered to permit curving of the coated flexible sheet. The coating thickness can range from a single molecule layer to several micrometer. The optimal thickness for the degree of curvature that is desirable (depending on the application) can be determined empirically. The degree of curvature required in the use of the device that comprises the flexible biochip can also be adjusted (for example, by adjusting spool diameter, if the substrate is to be wound around a spool, or by adjusting tube diameter, if the substrate is to form a tube structure) to accommodate the coating if necessary.

The coating can be applied in any appropriate way: vapor deposition, dipping, soaking, direct application, spraying, “painting”, chemical grafting etc. If the coating is a polymer, in some cases polymerization can be promoted on the substrate surface. The coating can be adhered to the substrate by absorption or chemical bonding. A glass coating can be applied, for example, by vapor deposition (if the substrate material is resistant to the heat required, or by allowing solgel (hydrolyzed siloxane) to polymerize to glass as it dehydrates on the substrate surface.

A flexible chip can be designed such that at least a portion of the surface of the chip is hydrophilic and at least a portion of the surface of the chip is hydrophobic. For example, a flexible chip fabricated with a hydrophobic substrate (such as a hydrophobic polymer) can be coated with a hydrophilic material in the area immediately surround the ion transport measuring means. The coating can be applied to both surfaces of the chip, or only the surface to which particles seal during use of the chip for ion transport measurement. In another example, a flexible chip fabricated with a hydrophobic substrate (such as a hydrophobic polymer) can be surface-modified to be hydrophilic in the area immediately surround the ion transport measuring means, such as by heating, oxidation, chemical treatment, etc. In yet another example, a hydrophilic chip substrate or coating on a substrate can be coated with a hydrophobic material or treated to make at least a portion of the chip surface apart from recording site areas hydrophobic.

The surface of the flexible chip or portions thereof can optionally be chemically treated, such as by using the methods described herein, to improve the electrical sealing properties of the chip. For example, at least a portion of the flexible chip can be treated with at least one salt or at least one base.

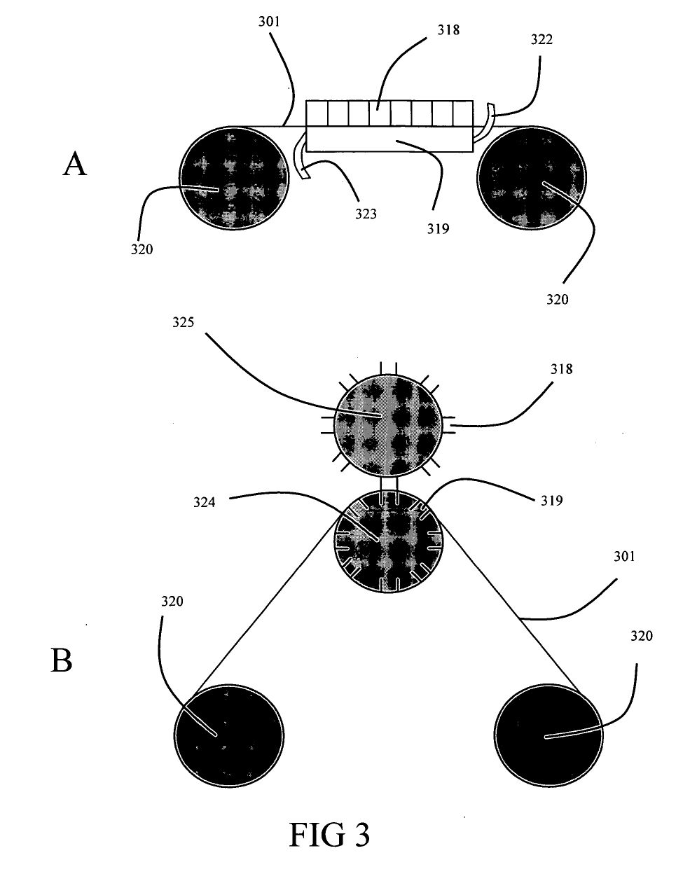

In one aspect of this embodiment of the present invention, an ion transport measuring device can be made using a flexible ion transport measuring biochip of the present invention that is wound around a spool (see FIG. 3). In this embodiment, the leading edge of the flexible biochip extends from the spool to either a second spool, or to a guide into which is inserted. The second spool or guide is positioned at a particular distance from the first spool such that an expanse of the flexible biochip is extended to be used for ion transport assays. The extended portion of the flexible biochip (301) can be essentially flat (FIG. 3A) or somewhat curved (FIG. 3B). Preferably, the extended portion of the flexible biochip comprises multiple ion transport measuring that matches the number of wells in multi-well plate for compound testing. Preferably, the extended portion of the flexible biochip comprises at least 8 ion transport measuring holes, more preferably, at least 12 ion transport measuring holes, even more preferably, at least 48 ion transport measuring holes, and yet more preferably, at least 96 ion transport measuring holes. For example, the extended portion of the flexible biochip can comprise 384 or 1536 ion transport measuring holes.

The present invention includes flexible ion transport measuring biochips made using these methods, and devices that include flexible ion transport measuring biochips.

An upper chamber piece can engage the upper side of the flexible biochip and a lower chamber piece can engage the lower side of the flexible biochip. In preferred aspects of these embodiments, the upper and lower chamber pieces are reusable, and the flexible biochip is single-use. In these aspects, the upper and lower chamber pieces reversibly engage the flexible biochip for ion transport assays. Upon completion of a set of assays, the upper and lower chamber pieces disengage and move away from the flexible biochip, a new section of the flexible biochip is unwound from the spool as the leading edge is pulled through guides and the old portion is optionally wound on a second spool, similar to camera film winding (in an alternative the used section can be pulled through guides and clipped off, similar to use of a tape dispenser). The new section of the flexible biochip that is unwound from the spool is to be used in the subsequent assay. The upper chamber piece and lower chamber piece (preferably one or both is reusable, but this is not a requirement of the present invention) now move to engage the new extended portion of the flexible biochip.

In FIG. 3A, the flexible biochip (301) has an extended portion between two spools (320) that engages an upper chamber piece having multiple upper chambers (318) and a lower chamber piece having a single lower chamber (319). An inflow conduit (322) and an outflow conduit (323) engage the lower chamber for directing solutions into and out of the chamber, and can also connect to pneumatic devices for applying pressure to the lower chamber.

In aspects in which the extended portion of the flexible biochip is somewhat curved, such as by curving against the surface of another, “chamber spool”, in which the contact surface of the spool also comprises the upper or lower chamber pieces, the upper and lower chamber pieces can be adapted to fit a curved biochip.

The upper chamber piece, the lower chamber piece, or both can be part of a chamber “wheel” in which multiple chamber pieces, each of which is used in performing a set of assays, can sequentially engage the flexible biochip. as shown in FIG. 3B. For example, a first set of assays can be performed using the first extended portion of the flexible biochip (301) and a first lower chamber piece (319) that is part of a lower chamber wheel (324) and can rotate below the surface of the flexible biochip. Upon completion of the first set of assays, the used portion of the flexible biochip (301) is pulled away from the wheel (324) as a new portion of flexible biochip (301) comes into proximity with the lower chamber wheel (324). During this period of time, the lower chamber wheel (324) rotates so that the used lower chamber piece (319) moves away from the assay site, and a new chamber piece comprising lower chambers (comprising measuring solution) also attached to the wheel engages the new extended portion of flexible biochip at the assay site. In the meantime, the used lower chambers can be washed as they turn with the wheel to be re-used with a new strip of the flexible biochip. In this depiction, an upper chamber wheel (325) provides upper chamber pieces (318) that also engage the flexible chip (301), and can rotate sequentially to engage the chip for ion transport assays and disengage the chip when a set of assays is complete, preferably to be washed and re-used in subsequent assays.

Various other upper and lower chamber configurations can be combined with the flexible biochip. For example, an upper chamber piece that engages the flexible biochip can have multiple upper chambers, such that each ion transport measuring hole is associated with a single upper chamber, and a lower chamber piece can also have multiple lower chambers, such that each ion transport measuring hole is associated with a single lower chamber. It is also possible to have a single lower chamber that accesses all of the ion transport holes used in an assay and multiple individual upper chambers. In other cases a single upper chamber that accesses all of the ion transport holes used in an assay and multiple individual lower chambers. Different chamber arrangements can have different electrode connections, connections to fluidic channels for the addition and removal of solutions and cells, and connection to pneumatic devices for sealing particles to ion transport measuring holes by the application of pressure.

In one preferred design, both the upper chamber piece and the lower chamber piece comprise multiple chambers that align with the extended portion of the flexible biochip such that each ion transport measuring hole is associated with a single upper chamber and a single lower chamber. In this design, cells, extracellular solutions, and compounds can be added to the top chambers either by individual conduits or by fluid dispensing systems. Pneumatic conduits connect with the lower chambers to produce high resistance seals. Electrodes can be provided in the reusable chamber pieces, or can be provided in fluid conduits or as part of the ion transport recording machinery that can be brought into electrical contact with the chambers through electrolyte bridges.

In yet another aspect of a flexible ion transport measurement biochip, the flexible biochip can form an at least partially tubular structure. The flexible biochip can form at least a portion of a tube. Where the flexible biochip does not form the complete circumference of a tube, the same flexible substrate material or a different material can form the remainder of the circumference of the tube. For example, the same flexible substrate material or a different material can form a basin or bottom surface of a trough-like structure that is continuous with the curved chip but can be at least in part flat or have a lesser degree of curvature. In this embodiment, the interior of the “tube” can form a single intracellular chamber, and an “upper” chamber piece can fit around the tube to provide upper chambers. In this aspect, cells, measuring solution (such as extracellular solution) and compounds can be added to individual upper chambers that can also contain, or be in electrical connection with, recording electrodes. The inner tube chamber can be a common chamber that has fluidic and pneumatic connections for providing measuring solutions and applying pressure for sealing of cells or particles to ion transport measuring holes. Preferably in this embodiment the lower chamber comprises or is in electrical connection with a reference electrode.

The present invention also includes a method of using a flexible biochip for measuring ion transport activity or properties. The flexible biochip can be part of a device in which sections of the flexible biochip are sequentially unwound for sequential sets of assays, or can be used as an at least partly curved surface.

The flexible biochip concept can be applied to not only ion transport assays, but also other high-throughput tests, in which a expanse of the biochip is used for testing at a time, where the top and optional the bottom surface of the biochip can be engaged in activities such as reagent delivery, detection, separation, etc.

Theta Tubing-Based Chip

Another aspect of the present invention is a method of making a multiplex ion transport measuring device using theta tubing. Either semicircular or rectangular theta tubing can be used, however, in some cases rectangular theta tubing can be preferred because the septum between the theta openings (referred to herein as “compartments”) is typically of a more uniform thickness in rectangular theta tubing. In this method, multiple segments of theta tubing can be stacked on top of one another or arranged side-by-side, where each segment comprises an ion transport measuring means (recording site).

The method comprises: providing at least two segments of theta tubing, each of which comprises an upper compartment and a lower compartment, where the upper compartment and lower compartment is separated by a glass septum; cutting an opening in the top of the theta tubing segments to provide access to the upper compartment; using the access at the top of the upper compartment to make at least one hole through the glass septum that separates the upper and lower compartments of each piece of theta tubing; and attaching the at least two segments of theta tubing one on top of another, such that the bottom compartment of a second theta tubing segment is on top of the upper compartment of a first theta tubing segment.

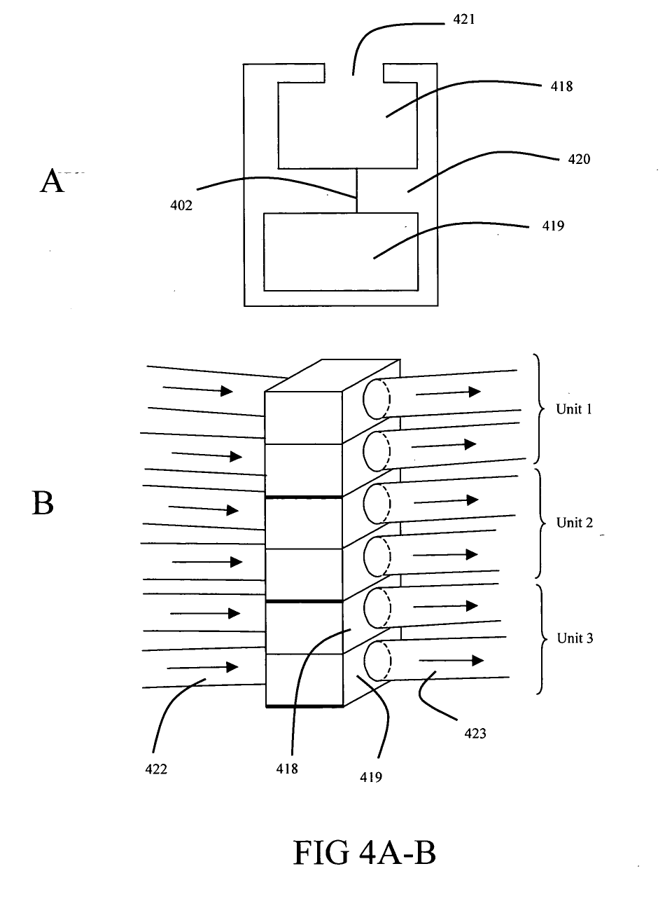

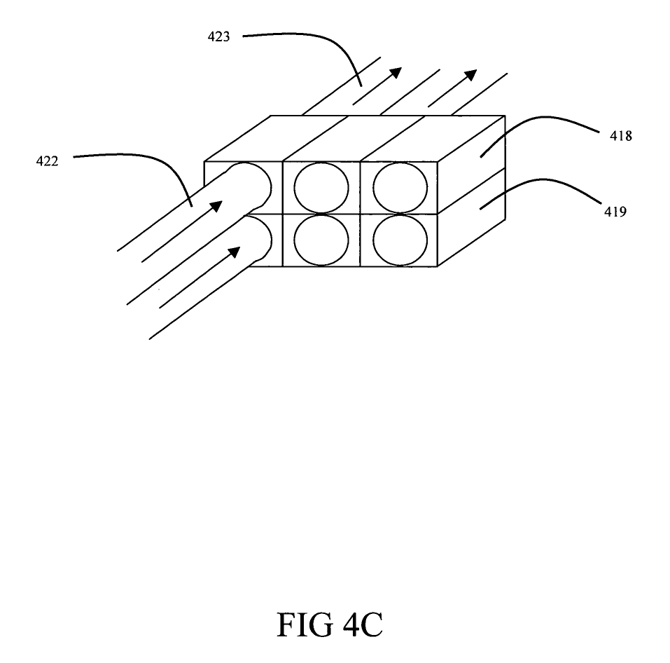

Preferably, openings cut in the top that are made to provide access for laser drilling or etching of the hole are sealed prior to stacking the theta tubing segments on top of one another. FIG. 4A depicts a theta segment having an upper compartment (418) and a lower compartment (419) in which a hole has been cut in the top (421) for laser access to drill an ion transport measuring hole (402) through the septum (420) of the segment. Rubber, polymers, or even glass can be used to close the opening using adhesives or heat for sealing. For example, the opening in the top of the top compartment can be sealed when the theta segments are stacked on top of one another, preferably by placing a gasket (such as a piece of flexible rubber, plastic, or silicone) over the opening and stacking the next theta segment on top of it. The gasket can be held in place by adhesives clamps, or f heat can also be used to attach the stacked units to one another. Sealing of the hole can be done such that a port is left in the top of the top chamber. In embodiments where the units are attached side-by-side, the port can be used for adding compounds or cells.

In the assembled device, each theta tubing segment comprises at least one (preferably one) ion transport recording site, and each theta tubing segment comprises an ion transport recording unit, having an upper chamber (upper compartment of the theta tubing segment) and a lower chamber (lower compartment or opening of the theta tubing segment). The multiple ion transport measuring units can be arranged vertically as depicted in FIG. 4B, with the upper chambers (418) and lower chambers (419) of each unit open on either side and connected to inflow conduits (422) on one side and outflow conduits (423) on the other side. In an alternative design, depicted in FIG. 4C, multiple ion transport measuring units can be arranged side-by-side, with the upper and lower chambers of each unit open each open on either side and connected to inflow conduits (422) on one side and outflow conduits (423) on the other side.

The open sides of each chamber are used to attach conduits for fluid flow, cell and compound delivery, and pneumatic control. In some preferred embodiments of the present invention, depicted in FIGS. 4B and 4C, individual conduits for providing extracellular solution, compounds, and cells, are attached to one side of each upper compartment of the theta structure, and individual conduits lead out of each upper chamber at the opposite side of the theta structure. In these designs, individual conduits providing intracellular solution can be attached to one side of each lower compartment of the theta structure, and individual conduits for outflow of intracellular solution lead out of each lower chamber at the opposite side of the theta structure. Pressure can be applied either from the intracellular inflow conduit or the intracellular outflow conduit.

Many different arrangements are possible for providing solutions, compounds, cells or particles, and pressure to a theta multiplex ion transport measuring device. For example, cells can be introduced to the lower chamber, and pressure for sealing of cells can be applied to the upper chamber. Conduits can be arranged in any way that can provide pressure for particle sealing and fluid flow for the addition of solutions, compounds, and particles such as cells.

In making the device, commercially available theta tubing can be used. The glass tubing can be cut into segments of any size that will allow the segment to function as ion transport measuring unit. For example, in some preferred embodiments, the segments can be from about 0.1 mm to about 80 mm in width, more preferably from about 1 mm to about 10 mm in width. The volumes of the upper and lower chambers of the units can be the same or different. Preferably, the extracellular chamber has internal measurements of at least 20 microns by 20 microns, and the intracellular chamber has internal measurements of at least 10 microns by 10 microns.

Dimensions of the ion transport measuring through holes that are made (for example, by laser drilling or etching) into the theta separator segments are preferably from about 0.3 to about 8 microns in diameter. The ion transport measuring holes can also include etched or laser drilled counterbores, as described previously in this application.

As described herein, the surface of the theta segment can be treated or coated to promote sealability of the surface as described previously in this application.

From two to 100 or more theta segments can be attached in vertical or parallel orientation (see FIGS. 4B and 4C). Attachment can be through the use of adhesives, gaskets, and the like. As mentioned above, the opening in the upper chamber can be sealed before or during attachment of the units. Conduits for the addition of solutions, cells, and compounds, and for the application of pressure can be attached both open ends of the chamber in any functional way, and can also use gaskets, adhesive, adaptors, etc. Electrodes, if provided within the chambers, can be inserted into chambers before or after assembling the multiplex structure.

Electrodes can be situated within upper and lower chambers of the segments. Alternatively, for a theta multiplex device, an electrode provided external to a chamber can be in electronic contact with one or more upper chambers through an electrolyte (solution) bridge. For example, one or more electrodes can be provided in one or more conduits leading to one or more upper chambers of the device, or provided as part of the ion transport recording machinery (signal source/amplifier) such that the electrode or electrodes are in electrical contact with an ion transport measuring solution. Similarly, one or more electrodes can be provided in one or more conduits leading to one or more lower chambers of the device, or provided as part of the ion transport recording machinery (signal source/amplifier) such that the electrode or electrodes are in electrical contact with an ion transport measuring solution. In some preferred embodiments of the present invention in which a device is used for whole cell ion transport measurement, the upper chamber of each theta ion transport measuring unit is the “extracellular chamber” that comprises or is in electrical contact with a reference electrode. In this case, multiple upper chambers can optionally be in electrical contact (for example, through conduits that provide solution bridges) with a single reference electrode.

Many other electrode arrangements are possible, however, including but not limited to a single reference electrode in electrical contact with multiple lower chambers (which can be “intracellular” or “extracellular” chambers of the units), individual reference electrodes for each lower chamber, individual reference electrodes for each upper chamber, etc. Recording electrodes can also be provided within chambers or in electrical contact (for example, through conduits that provide solution bridges) with chambers.

The present invention also includes ion transport measuring devices made using the methods of the present invention. These ion transport measuring devices comprise at least two attached theta tubing segments, wherein the theta separator segment of each of the theta tubing segments comprises an ion transport measuring hole. Preferably, the upper and lower chambers of each theta tubing segment comprises or is in electrical contact with at least one electrode. Preferably, a theta ion transport measuring device comprises conduits that attach to upper and lower chambers of each theta tubing segment. The theta ion transport measuring device can comprise any functional arrangement of electrodes or electrical connections to electrodes, and any functional arrangement of fluidic and pneumatic structures (such as conduits, valves, and can connect systems for controlling fluid flow and pressure (for example, pumps), and electronic equipment for ion transport measurement.

For example, the lower chamber of a theta ion transport measuring unit preferably engages an inflow conduit on one side of the lower chamber and an outflow conduit on the opposite side of the lower chamber. A lower chamber can include or contact a lower chamber electrode that is preferably introduced into the lower chamber via an inflow or the outflow conduit. The lower chamber electrode in this embodiment can be, for example, a wire electrode inserted into the conduit. The electrode can be a common electrode (that contacts more than one lower chamber) or an individual electrode, in which each lower chamber contacts or comprises its own electrode. An individual lower chamber electrode can also be positioned in a lower chamber. A lower chamber common or individual electrode can also be part of a separate part of an apparatus used for ion transport measurement (such as for example, a signal amplifier) that during use of the device, is positioned such that it is in electrical contact with one or more lower chambers. This can be achieved, for example, by putting the one or more lower chamber electrodes in contact with a salt bridge (such as a solution-filled conduit) that engages the lower chamber. A lower chamber also preferably engages at least one conduit that provide pneumatic control of the ion transport recording unit. For example, the outflow conduit of a lower chamber can be connected to a pressure source such as a pump or syringe.

The upper chamber of a theta ion transport measuring unit preferably also engages an inflow conduit on one side of the lower chamber and an outflow conduit on the opposite side of the lower chamber. An upper chamber can include or contact an upper chamber electrode that is preferably introduced into the lower chamber via an inflow or the outflow conduit. The upper chamber electrode in this embodiment can be, for example, a wire electrode inserted into a conduit that leads to the chamber. The electrode can be a common electrode (that contacts more than one upper chamber) or an individual electrode, in which each lower chamber contacts or comprises its own electrode. An individual upper chamber electrode can also be fabricated into or positioned in an upper chamber. An upper chamber common or individual electrode can also be part of a separate part of an apparatus used for ion transport measurement (such as for example, a signal amplifier) that during use of the device, is positioned such that it is in electrical contact with one or more lower chambers. This can be achieved, for example, by putting the one or more upper chamber electrodes in contact with a salt bridge (such as a solution-filled conduit) that engages the lower chamber.

The present invention also includes methods of using ion transport measuring devices comprising at least two attached theta tubing segments to measure at least one ion transport activity or property of at least one particle (such as a cell). Methods of ion transport measurement are well known in the art and also described herein.

The present device can be used for any type of ion transport measurement, including whole cell, single channel, outside-out patch and inside-out patch recording. The multiplex theta device can be used for testing the effect of known and unknown compounds on ion transport activity of cells and particles.

Upper Chamber Designs

Flow-Through Upper Chamber

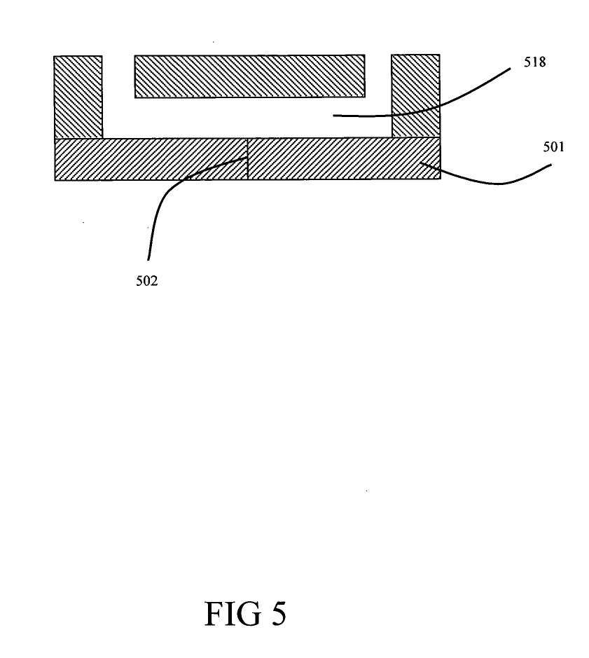

Another aspect of the present invention is a device for ion transport measurement that comprises a chip having at least one ion transport measuring hole and at least one upper chamber, where the one or more upper chambers comprise at least two openings, in which one of the openings is on one side of the one or more ion transport measuring holes and another of the openings is on the other side of the one or more ion transport measuring holes. In this device, a simple version of which is depicted in FIG. 5, the upper chamber (518) is a “flow-through” chamber that is accessed by at least one ion transport measuring hole (502) through a chip (501).

Fluids such as solutions and suspensions (for example, measuring solutions, wash solutions, samples comprising particles such as cells, or compound solutions) can be added through a first opening on one end of the flow-through chamber and removed from the chamber via an opening on another end of the flow-through chamber. Fluid flow through the chamber can be provided by pumps or syringe mechanisms, and preferably the flow rate is regulable. Preferably, fluid flow into and out of the chamber is via inflow and outflow conduits that engage the openings at either end of the chamber. Preferably, fluid flow into and out of the chamber can also be controlled by valves that can permit flow into or out of the chamber, or close off flow into or out of the chamber.

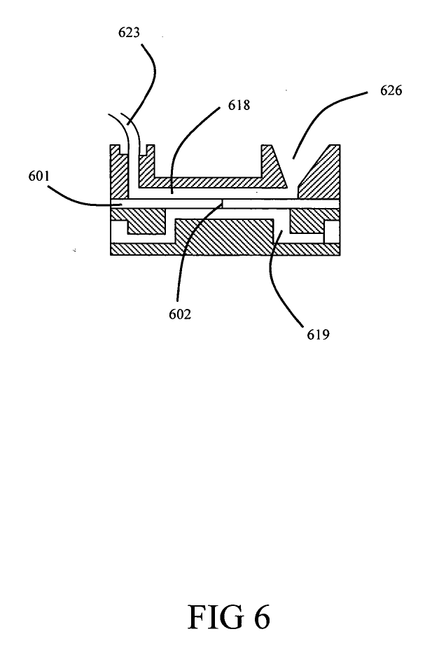

In some embodiments of this device, one of the two or more openings is directly or indirectly connected to a reservoir at its end where cells and, potentially, compounds can be added to the upper chamber such as by a fluidic system or pipette. For example, the device depicted in FIG. 6 has a flow-through upper chamber (618) and a flow-through lower chamber (619) separated by a chip (601) that comprises an ion transport measuring hole (602). The flow-through upper chamber (618) connects to a conduit (623) at one end of the chamber, and a reservoir (626) for the addition of sample at the other end of the chamber. The device shown in FIG. 6 can be a single-unit device, or a device of the present invention can comprise multiple ion transport recording units, each comprising a flow-through upper chamber and a flow-through lower chamber connected by an ion transport measuring means.

The one or more upper chambers of a device of the present invention can comprise an electrode, or, during use of the device, can be in electrical contact with an electrode that can be part of the signal amplifier machinery or can be provided in tubing leading to the chamber.

In preferred embodiments of this aspect of a device of the present invention, a device has a single upper chamber with two openings, one on either side of the one or more ion transport measuring holes, such that measuring solution buffers, or compound containing solutions (such as Extracellular Solution, ES) can flow through the upper chamber. For example, measuring solution can be pumped through the upper chamber to fill or wash the chamber. In embodiments in which an opening directly or indirectly accesses a reservoir outside the chamber, particles such as cells and compounds can optionally be added to the upper chamber via the reservoir. In the alternative, solutions, particles, or compounds can be added to the upper chamber at an opening that does not provide access to a reservoir. Preferably, at least a portion of the upper surface of a flow-through upper chamber device is transparent, so that cells in the upper chambers can be viewed microscopically.

A device of the present invention can have multiple flow-through upper chambers, each of which is accessed by a single ion transport measuring hole, or can have multiple flow-through upper chambers, each of which is accessed by multiple ion transport measuring holes, or, in some preferred embodiments, can have a single flow-through upper chamber that is accessed by multiple ion transport measuring holes.

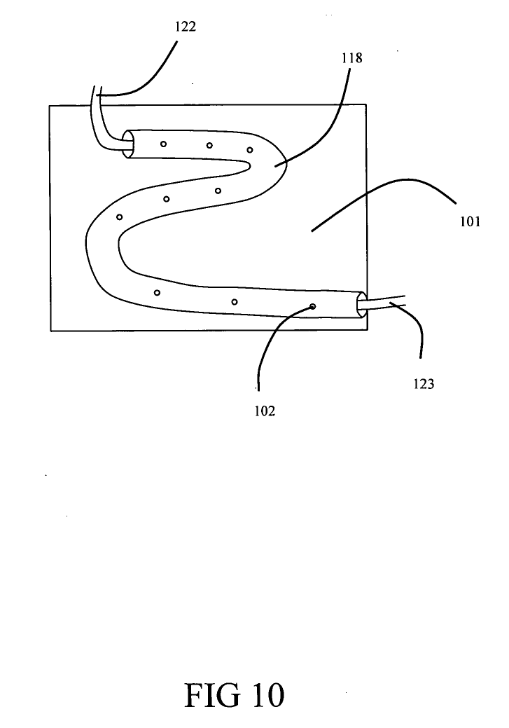

One preferred embodiment is a device having a chip that comprises two or more ion transport measuring holes that access a single flow-through upper chamber. In this embodiment, the flow-through chamber can be arranged as a channel having an inlet at one end, two or more ion transport measuring holes positioned in a linear fashion along the course of the channel, and an outlet at the opposite end. An upper chamber channel can be straight or curved, and a chip can optionally engage more than one flow-through upper channel. FIG. 10 depicts a device comprising a chip (101) that has multiple ion transport measuring holes (102) that access a flow through upper chamber channel (118) that engages an inflow conduit (122) at one end of the channel (118) and an outflow conduit (123) at the other end of the channel (118). A chip of a device that comprises a flow-through upper chamber channel can comprise one or more holes, preferably four or more holes, more preferably 16 or more holes, and more preferably yet 48 or more holes, all of which can access a single channel. A device comprising an upper chamber channel that accesses multiple ion transport measuring holes of a chip preferably also comprises multiple lower chambers, each of which accesses on of the ion transport measuring holes of the chip. Preferably, the upper chamber comprises, contacts, or, during use of the device, is in electrical contacts with an electrode that serves as a common upper chamber electrode, and each of the multiple lower chambers comprises, contacts, or, during use of the device, is in electrical contacts with an individual electrode that serves as a recording electrode. Preferably, the lower chambers have inflow and outflow conduits for the addition and removal of measuring solutions, and are connected to at least one pneumatic device for applying pressure through the lower chambers to seal particles in the upper chamber channel to ion transport measuring holes.

In some preferred designs, a flow-through upper chamber ion transport measuring device comprises an upper chamber piece that forms at least the walls of the one or more flow-through upper chambers, and a chip that comprises at least one ion transport measuring hole that forms the bottom of the chamber. The upper chamber piece can reversibly or irreversibly engage the chip such that a fluid impermeable seal is formed between the upper chamber piece and the chip. It is also possible to have an upper chamber piece that forms the walls of at least one upper chamber and also comprises lower surfaces of the chambers. In this case, the upper chamber piece itself comprises one or more ion transport measuring holes that are machined, etched, or drilled into the bottom surface of the one or more upper chambers, where the bottom surface of the upper chamber piece serves as the “chip” or substrate where particle sealing takes place.

Preferably, the upper chamber piece also forms the tops of the chambers. In one alternative, the upper chamber piece can reversibly or irreversibly engage a top piece that forms the top of the one or more upper chambers. In some preferred embodiments at least a portion of the top surface of the chambers is transparent, allowing cells or other particles being assayed to be viewed microscopically. In some embodiments, however, the upper chamber or chambers of a device of the present invention can be open at the top.

An upper chamber piece can be made of any suitable material, including but not limited to, one or more plastics, one or more polymers, glass, one or more ceramic materials, coated metals, or combinations thereof. Nonlimiting examples of plastics that can be used in the manufacture of upper chamber pieces include, but are not limited to polyallomer, polypropylene, polystyrene, polycarbonate, cyclo olefin polymer, polyimide, paralene, PDMS, polyphenylene ether/PPO, Noryl®, and Zeonor®. Glass and transparent polymers are preferred transparent materials, with transparent polymers such as polycarbonate and polystyrene having the advantage of easier manufacture.

The design and dimensions of a flow-through upper chamber piece, as well as the dimensions of upper chambers, can vary according to the preferences of the user and are not limiting to the present invention. For example, the volumetric capacity of the one or more flow-through upper chambers formed by the piece can vary from about ten microliters to about 100 milliliters or more, depending in part on the number of ion transport measuring holes that access an upper chamber.

The upper chamber or chambers of a device of the present invention can optionally comprise one or more electrodes. Electrodes can be attached to or fabricated on the walls of the chamber or chambers, or can be attached to or fabricated on the bottom surface of an upper chamber, such as on the surface of a chip that forms the bottom of the upper chamber or chambers. In preferred embodiments in which a flow-through upper chamber is accessed by more than one ion transport measuring hole, an upper chamber can comprise a single electrode that can be used as a common reference electrode during ion transport measurement assays. In alternative designs, an electrode or electrodes can be in electrical contact with the one or more upper chambers during use of a device. In these designs, an electrode can be provided in a conduit leading to an upper chamber, or can be part of another machine or device (such as, for example, a signal amplifier) that is connected through a salt bridge (for example, measuring solution in a conduit connected to the device) to the upper chamber.

Electrodes can comprise conductive materials such as metals that can be shaped into wires. Various metals, including aluminum, chromium, copper, gold, nickel, palladium, platinum, silver, steel, and tin can be used as electrode materials. For electrodes used in ion channel measurement, wires made of silver or other metal halides are preferred, such as Ag/AgCl wires.

In using device of the present invention having one or more flow-through upper chambers, the device can engage a lower chamber piece. The lower chamber piece can be in the form of a tray or tank, and preferably has at least one inlet and at least one outlet for allowing measuring solution (such as IS, intracellular solution) to flow into the chamber and for the application of pressure for sealing particles to the one or more ion transport measuring holes. In some preferred embodiments the lower chamber is also a single flow-through channel, with an opening at one end for the introduction of solutions such as measuring solution, and an opening at the other end for outflow of solutions, and preferably, connection to pneumatic devices for applying pressure to seal particles to the one or more ion transport measuring holes.

The present invention also includes ion transport measuring devices with one or more flow-through upper chambers that also comprise one or more lower chambers. In these devices, each of the one or more lower chambers is accessed by one or more ion transport measuring holes of the device. In preferred designs, a chip that comprises one or more ion transport measuring holes forms the upper surface of the one or more lower chambers. Preferably, at least one pneumatic device is connected to one or more lower chambers of a device of the present invention, such as, for example, a pump or syringe that is connected to a lower chamber via a conduit. The pneumatic device can provide pressure control to seal a particle in an upper chamber in fluid communication with the lower chamber to an ion transport measuring hole of the chip.

Several designs of ion transport measuring devices that comprise flow-through upper chambers and at least one lower chamber are possible. For example, a device can have multiple flow-through upper chambers, each of which is accessed by one ion of multiple transport measuring hole, and a single lower chamber, where the single lower chamber is in fluid communication with multiple upper chambers via multiple ion transport measuring holes. In an alternative embodiment, a single flow-through upper chamber (such as the upper chamber channel depicted in FIG. 10) is accessed by multiple ion transport measuring holes, each of which accesses one of multiple lower chambers. In another design, a device has a chip comprising one or more ion transport measuring holes, one or more flow-through upper chambers, and one or more lower chambers (preferably also having a flow-through design). Each of the flow-through upper chambers is accessed by a single ion transport measuring hole that also accesses a single lower chamber. For example, FIG. 6 depicts a biochip having a single ion transport measuring hole, a flow-through upper chamber and a flow-through bottom chamber, where the cells can be viewed through the top of the upper chamber using a microscope. The invention also includes devices having multiple upper chambers, and multiple lower chambers, where each of the multiple upper chambers is in fluid communication with one of the multiple lower chambers via one of multiple ion transport measuring holes.

In preferred embodiments of aspects of the present invention having a chip comprising one or more ion transport measuring holes and at least one flow-through upper chamber, an upper chamber piece that forms at least the walls of one or more upper chambers is reversibly or irreversibly attached to the upper surface of a chip that forms the bottoms of the upper chambers, and a lower chamber piece that forms at least the walls of one or more lower chambers is reversibly or irreversibly attached to the lower surface of a chip that forms the tops of the upper chambers. The lower chamber piece can comprise any suitable material, including but not limited to, one or more plastics, one or more polymers, glass, one or more ceramic materials, coated metals, or combinations thereof. Nonlimiting examples of plastics that can be used in the manufacture of lower pieces include, but are not limited to polyallomer, polypropylene, polystyrene, polycarbonate, cyclo olefin polymer, polyimide, paralene, PDMS, polyphenylene ether/PPO, Noryl®, and Zeonor®. Glass and transparent polymers are some preferred transparent materials, with transparent polymers such as polycarbonate and polystyrene having the advantage of easier manufacture.

The design and dimensions of a lower chamber piece, as well as the dimensions of lower chambers, can vary according to the preferences of the user and are not limiting to the present invention. For example, the volumetric capacity of the one or more upper chambers formed by the piece can vary from about one microliter to about 100 milliliters or more, depending in part on the number of ion transport measuring holes that access a lower chamber.

The lower chamber or chambers of a device of the present invention can optionally comprise one or more electrodes. Electrodes can be attached to or fabricated on the walls or lower surface of the chamber or chambers, or can be attached to or fabricated on the bottom surface of a chip that forms the upper surface of a lower chamber or chambers. In preferred embodiments in which a flow-through lower chamber is accessed by more than one ion transport measuring hole, a lower chamber can comprise a single electrode that can be used as a common reference electrode during ion transport measurement assays. In alternative designs, an electrode or electrodes can be in electrical contact with the one or more lower chambers during use of a device. In these designs, an electrode can be provided in a conduit leading to a lower chamber, or can be part of another machine or device (such as, for example, a signal amplifier) that is connected through a salt bridge (for example, measuring solution in a conduit connected to the device) to a lower chamber.

Upper Chamber Separator Unit

In yet another aspect of the present invention, an ion transport measuring device comprises a chip comprising two or more ion transport measuring holes; a common upper chamber positioned above the chip, such that the chip forms the bottom of the common upper chamber and the two or more ion transport measuring holes of the chip access the common upper chamber, and an upper chamber separator unit that can be reversibly lowered onto the chip to separate the common upper chamber into multiple individual upper chamber compartments that are in fluidic isolation from one another. The upper chamber separator unit comprises multiple separator segments that contact the upper surface of the chip within the upper chamber to form at least a portion of the walls of the multiple individual upper chamber compartments, each of which is in register with an ion transport measuring hole of the chip.

The physical separator can reversibly fasten on to the substrate. The upper chamber separator can comprise any fluid-impermeable material, and preferably comprises a compressible material (such as a polymer) where the separator segments contact the surface of the chip to seal against the chip so that the separator forms fluid-impermeable separated upper chamber compartments. Preferably, the upper chamber separator comprises one or more top pieces attached to the separator segments to serve as tops or lids of the upper chamber compartments. Preferably, the one or more top pieces comprise openings that can be used, for example, for compound delivery to the upper chambers, or for electrode contact with the upper chamber compartments. At least a portion of a top piece can optionally be transparent so that particles such as cells in the upper chamber compartments can be viewed microscopically.

The devices of the present invention that comprise physical separator units for forming chambers can comprise ion transport chips as they are known in the art and described herein, including, for example, planar chips, flexible chips, and MCP chips. Chips used in the devices can be treated, such as using methods described herein, to improve their sealing properties. For example, a chip used in a device of the present invention can be made more electronegative by, for example, chemically treating the chip with at least one salt or at least one base.

The upper chamber of a device can comprise an electrode. For example, an electrode layer that can serve as a common upper chamber electrode can be fabricated onto the upper surface of the chip. In this embodiment, the device comprises or engages multiple lower chambers, each of which comprises or contacts an individual electrode. In an alternative design, the upper chamber separator unit can comprise either a common electrode or individual electrodes that contact each upper chamber compartment when the separator unit is positioned on the chip. The one or more electrodes can be attached to the upper chamber separator, or inserted through conduits that engage the upper chamber separator, or can be in electrical communication with the upper chamber compartments via one or more conduits that engage the upper chamber separator.

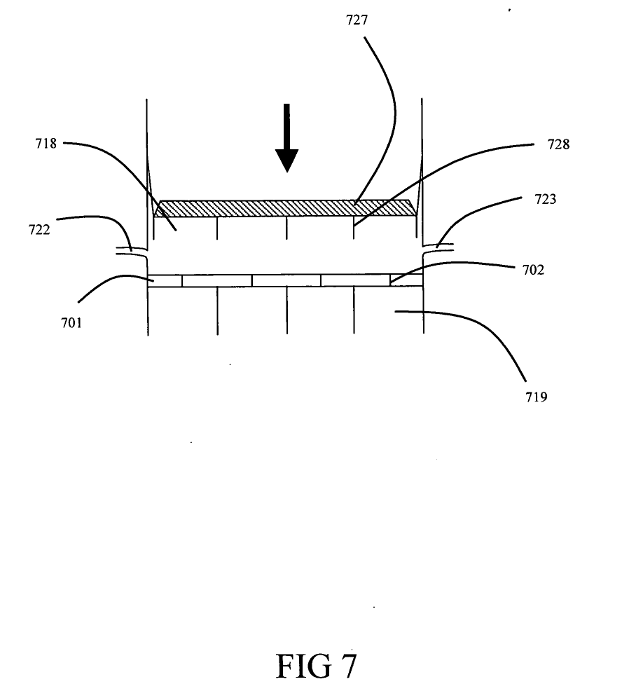

Preferably, a device with an upper chamber separator unit further comprises or engages one or more lower chambers. For example, a device can further comprise a common lower chamber positioned beneath the chip, where the chip forms the top of the common lower chamber and the two or more ion transport measuring holes access the lower chamber. In an alternative embodiment, the device can further comprise two or more lower chamber positioned beneath the chip, where the chip forms the tops of the multiple lower chambers, each of which is aligned with a single ion transport measuring hole. In the embodiment depicted in FIG. 7, the ion transport measuring device comprises a chip (701) having multiple ion transport measuring holes (702), each of which accesses an independent lower chamber (719). The device has a common flow-through upper chamber (718) that is accessed by multiple ion transport measuring holes (702). The upper chamber engages an inflow counduit (722) and an outflow conduit (723). The device also comprises an upper chamber separator unit (727) comprising separator segments (728) that, when the separator unit is lowered onto the chip within the common upper chamber, form independent upper chamber compartments, each of which is accessed by a single ion transport measurement hole of the chip.

Preferably, the one or more lower chambers of a device that comprises an upper chamber separator are connected to at least one pneumatic device for the application of pressure to the one or more lower chambers for sealing particles in the upper chamber to ion transport measuring holes. For example, the one or more lower chambers can comprise a conduit that can connect to a pump or syringe for applying negative pressure to the one or more lower chambers.

Where the upper chamber separator unit provides individual electrodes, the device can optionally have a common lower chamber that comprises or contacts a common electrode or multiple lower chambers that comprise or contact a common electrode or individual electrodes.

In embodiments where the device comprises multiple lower chambers, each of the multiple lower chambers can each comprise or contact an individual electrode. The lower well electrodes can optionally be provided by a separate adaptor plate that can reversibly engage the lower wells and can also optionally comprise connections to pneumatic devices for pressure control. In this case, a common electrode (for example, an electrode that traverses the upper surface of the chip) can contact or be positioned within the upper chamber or be brought into electrical contact with the multiple upper chamber compartments.

The upper chamber separator unit can be lowered into the upper chamber (which can be in the form of a tank with ion transport measuring holes in the bottom) after measuring solution and cells (or other particles) have been introduced into upper chamber and preferably after particles have been sealed to the ion transport measuring holes. Preferably, the upper chamber is a flow-through upper chamber that connects to inflow and outflow conduits that can be used for adding measuring solutions and cells before the separator unit is lowered onto the chip, and for washing the chamber after assays have been completed and the separator unit has been raised off the chip.

In embodiments in which the separator unit comprises multiple electrodes, each of the separate electrodes can contact a separate chamber when the separator engages the chip. In these embodiments, the device can also comprise or engage a common lower chamber that can comprise or contact a common electrode that can be used as a reference electrode. In an alternative, a common upper chamber electrode can be built onto the upper surface of the chip.

After cells have sealed to the chip and the separator unit has formed separate upper chambers, compounds can be added to the individual upper chambers, either by conduits or fluid dispensing systems. In embodiments in which the separator unit also forms the tops of upper chamber compartments, solution dispensing can occur through openings in the separator unit. Ion transport recording can then be performed using upper chamber recording electrodes and a bottom chamber reference electrode, or preferably, a common upper chamber reference electrode and recording electrodes that contact the lower chambers of the device.

FIG. 7 depicts an ion transport measuring device of the present invention having a common upper chamber that is divided into separate upper chamber compartments by an upper chamber separator unit. In FIG. 7A, a device is shown having a chip (701) comprising ion transport measuring holes (702) and a common flow-through upper chamber (708) with an inlet (709) and an outlet (710). The device also has multiple lower chambers (711) in register with the ion transport measuring holes (702).

Devices Comprising Chips Having Built-on Upper Wells

In a related aspect, the present invention comprises a chip comprising at least one ion transport measuring hole, in which the chip comprises at least one upper well on its top surface surrounding an ion transport measuring hole and the upper well is built onto the chip. In one preferred embodiment, the well comprises a layer of wax.

The chip can comprises any hard material such as metals, ceramics, polymers, inorganic and organic hybrid materials, plastics, silicon dioxide, or glass, and the ion transport measuring holes can be etched, laser drilled, cut, punched out, or bored into the material. In preferred embodiments, the chip is a glass chip and the ion transport measuring holes are laser drilled. Preferably, the chip is surface-treated, such as by using methods described herein.

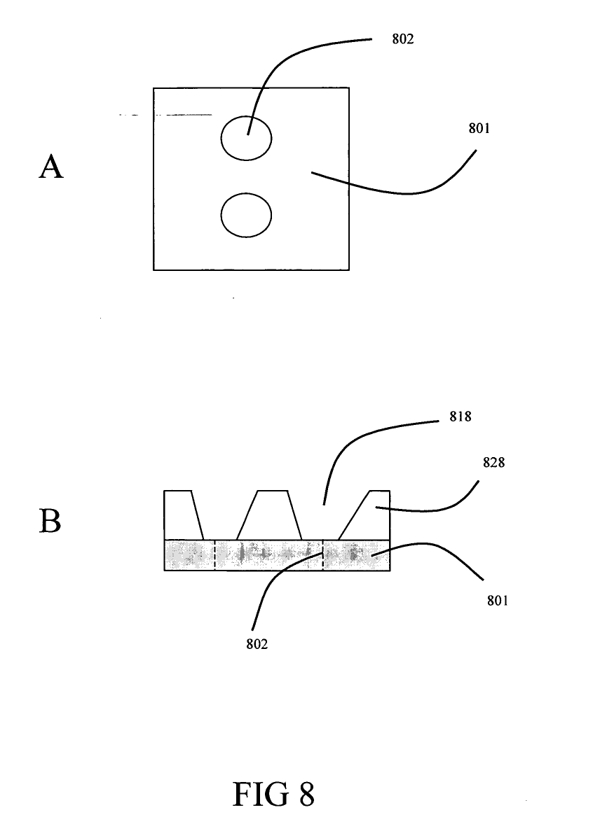

Preferably, the wax on the upper surface of the chip forms individual wells, after ion transport measuring holes is created through the chip. FIG. 8A depicts an overhead view of a chip (801) having two upper chambers (818). FIG. 8B depicts the chip (801) in cross section, showing the upper chambers (818) made of wax wells (828) surrounding ion transport measuring holes (802).

As in the previous embodiment, during use, the chip is assembled with one or more structures to form an ion transport measuring device. In this case, however, the chip comprises upper chamber wells on its surface, and the chip engages a structure that preferably comprises as upper piece top surface that forms the top of the upper wells, as well as a lower chamber piece. The wax-formed upper chamber structures on the upper surface of the chip are at least somewhat compressible, allowing sealing of the upper chamber structures to the upper piece surface when the device is assembled.

The upper piece top surface that engages the chip can also include conduits and, optionally, electrodes that can connect with the one or more upper chambers of the device when the device is assembled.

Preferably, the chip comprises multiple wax-formed upper chambers and the lower chamber piece it assembles with has multiple isolated lower chamber wells, but other designs are possible. For example, the chip can have a single wax-formed upper chamber, and can be assembled with a structure that comprises multiple isolated lower chambers. In an alternative, the chip comprises multiple wax-formed upper chambers and the lower chamber piece has a single common lower chamber.

In preferred embodiments, the chip having wax-formed upper chambers is single-use and disposable, and the lower chamber piece and the structure that comprises the upper piece top surface, as well as associated electrodes (which can be part of the signal amplifier machinery or electrodes that can be attached or connected to the wells), are reusable.

Another material that can be used for forming wells on the surface of a biochip is SU-8. SU-8 is a photo-curable epoxy oligomer, commonly used for computer chip manufacture. To make one or more wells on the surface of a chip using SU-8, the liquid form of the oligomer is distributed on the surface of the chip. A mask is used to pattern one or more wells. Light induces polymerization of SU-8 in areas not covered by the mask. After polymerization, the unpolymerized SU-8 is washed away to leave chamber walls that comprise SU-8 polymer.

Chip with O-Ring Upper Chambers

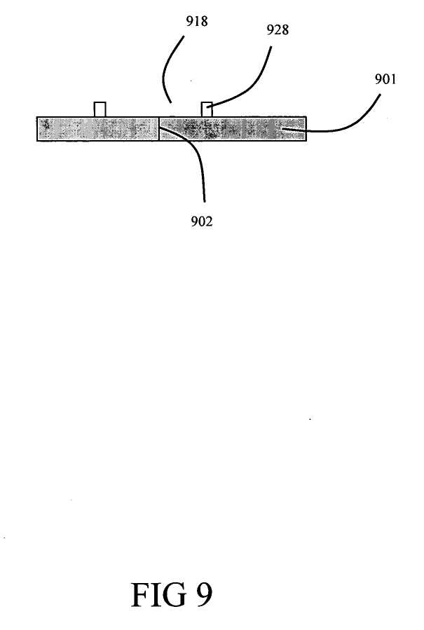

In a related aspect of the present invention, a chip comprising at least one ion transport measuring hole is provided with at least one O-ring that forms an upper chamber around the at least one ion transport measuring hole. FIG. 9 is a cross-sectional view showing a chip (901) having an ion transport measuring hole (902) surrounded by an O-ring (928) that forms an upper chamber (918).

The chip having O-ring upper chambers can be assembled with at least one structure to form an ion transport measuring device.

The chip can comprises any hard material such as metals, ceramics, polymers, plastics, silicon dioxide, or glass, and the ion transport measuring holes can be etched, laser drilled, cut, punched out, or bored into the material. In preferred embodiments, the chip is a glass chip and the ion transport measuring holes are laser drilled. Preferably, the chip is surface-treated to increase its sealing properties, such as by using methods described herein.

To assemble an ion transport measuring device, the chip preferably engages a structure that preferably comprises as upper piece top surface that forms the top of the upper wells that can be reversibly attached to the top of the chip, as well as a lower chamber piece that can be reversibly attached to the bottom of the chip. The upper chamber O-ring structures on the upper surface of the chip are at least somewhat compressible, allowing sealing of the upper chamber structures to the upper piece surface when the device is assembled. The O-ring can also be sealed to the top of chip surface using an adhesive.

The upper piece surface can also include conduits and, optionally, electrodes that can connect with the one or more upper chambers of the device when the device is assembled.

Preferably, the chip comprises multiple O-ring upper chambers and the lower chamber piece has multiple isolated lower chamber wells, but other designs are possible. For example, the chip can have a single O-ring upper chamber, and can be assembled with a structure that comprises multiple isolated lower chambers. In an alternative, the chip comprises multiple O-ring upper chambers and the lower chamber piece has a single common lower chamber well.

In preferred embodiments, the chip having O-ring upper chambers is single-use and disposable, and the upper piece surface and lower chamber piece, as well as associated electrodes (which can be part of the signal amplifier machinery or electrodes that can be attached or connected to the wells), are reusable.

Fluidic Systems

Overhead Delivery of Solutions to Ion Transport Recording Sites in Flow-Through Upper Chambers

The present invention provides novel fluidic systems for delivering solutions, compounds, and particles (such as cells) to compartments of ion transport measuring devices. These fluidic systems can be applied to a number of chip designs and device designs that may vary in their structures, electrode arrangements, or pressure systems.

In some preferred embodiments of the novel fluidic systems of the present invention, an ion transport measuring device comprises a biochip comprising two or more ion transport measuring holes, and a flow-through upper chamber positioned above the biochip that is accessed by the two or more ion transport measuring holes. The two or more ion transport recording holes access the one or more upper chambers at ion transport measurement recording sites. The device further comprises a fluid delivery system comprising two or more fluid delivery units, each of which can be aligned directly over one of the two or more ion transport recording sites each of which encompasses an ion transport measurement hole. Solutions (including test compound solutions) can be added to ion transport recording sites that surround the ion transport recording holes through the fluid delivery units that can be positioned over the ion transport recording sites. Preferably, the fluid delivery units can align directly over and in close proximity to the ion transport measuring holes of the chip. The fluid delivery units can comprise, for example, pipets, conduits, pipes, tips, or sonic actuators. The diameter of the dispensing opening of a fluid delivery unit is preferably less than half the distance between ion transport measuring holes of the chip. Preferably, the diameter of the dispensing opening of a fluid delivery unit is between about 50 microns and 5000 microns, more preferably between about 200 microns and about 2000 microns.

The fluid delivery units are preferably part of a fluid delivery array (for example, a multichannel pipet array) of a fluidics block that can be reversibly positioned over the upper chamber such that individual delivery units of the array align with ion transport measurement recording sites of the device. Preferably, positioning of the delivery units is automated.

The flow-through upper chamber comprises at least one inlet and at least one outlet that can allow for fluid flow through the chamber. Chamber solutions (such as measuring solutions such as ES) and cells or compounds can be added via an upper chamber inlet. An electrode, such as a reference electrode, can optionally be provided within or, during use of the device, in electrical connection with the flow-through upper chamber.

In some preferred embodiments, a flow-through upper chamber of a device of the present invention comprises an upper surface that comprises two or more openings, in which each of the two or more openings is aligned over one of the two or more ion transport recording sites. The openings provide access of the fluid delivery units to ion transport recording sites of an upper chamber. In other embodiments, a chamber does not have an upper surface, and each of the two or more fluid delivery units can be aligned directly over the one of the two or more ion transport measuring recording sites and solutions can be added via fluid delivery units that are positioned over ion transport recording sites.

These embodiments of the present invention that include ion transport measuring devices having flow through upper chambers and overhead delivery systems can comprise ion transport chips as they are known in the art and described herein, including, for example, planar chips, flexible chips, chips having hydrophobic modifications, and MCP chips. Preferably, a chip of a device having a flow-through upper chamber and an overhead delivery fluid system comprises two or more microwells, in which each of the two or more ion transport measuring holes of the chip is surrounded by a microwell on the upper surface of the chip. Such microwells can define ion transport measurement recording sites of the upper chamber of a device.

Preferably, a chip of a device having a flow-through upper chamber and an overhead delivery fluid system comprises four or more holes, more preferably 16 or more, more preferably yet 48 or more, and most preferably 96 or more.

Where feasible, chips used in the devices can be treated, such as using methods described herein, to improve their sealing properties. For example, at least a portion of a chip used in a flow-through, overhead fluid delivery device of the present invention can be treated to make the surface of an ion transport measuring means or surrounding an ion transport measuring means more electronegative. For example, at least a portion of a chip used in a device of the present invention can be treated with at least one salt or at least one base.

Preferably, the ion transport measuring device further comprises one or more lower chambers positioned below the chip in register with the two or more ion transport measuring holes of the chip. In preferred embodiments, the chip engages a lower chamber piece that comprises at least the walls of two or more individual lower chambers, such that each ion transport measuring hole of the biochip accesses its own lower chamber. The lower chambers preferably each comprise or contact an individual electrode, or during use of the device are in electrical connection with individual recording electrodes. The lower chambers also preferably are connected to one or more pumps or other pressure-generating devices, and engage conduits for the addition and removal of measuring solution. The lower well electrodes can optionally be provided by a separate adaptor plate that can reversibly engage the lower wells and can also optionally comprise connections to pneumatic devices for pressure control. In other embodiments, the device comprises a single common lower chamber that comprises, contacts, or, during use of the device, can be in electrical contact with a common electrode.

Various chamber and electrode designs can be used with these devices. For example, the upper surface of the chip can comprise microwells at the individual recording sites, and the upper surface of the chip can comprise a common reference electrode that is coated with a hydrophobic material except where it contacts the microwells. In this case, the device has multiple independent lower wells, each of which is associated with a single ion transport measuring hole. Each lower well comprises or contacts an independent electrode that can be used for ion transport recording.

In an alternative, the device can have a single bottom chamber that comprises or contacts a reference electrode. Individual recording electrodes can be provided in connection with the upper microwells. The individual upper chamber electrodes can be inserted into the microwells, for example. In one embodiment, the recording electrodes can be attached to the compound delivery system, such that positioning of the compound delivery system over the microwells can also serve to position and dip an electrode into the microwell.

During use of a device of the present invention having at least one flow-through upper chamber and an overhead solution delivery system, there is continuous flow of chamber solution (such as a measuring solution) through the upper chamber, in which the chamber solution enters through a chamber inlet and exits through a chamber outlet. A test solution, such as a compound solution, is added to a recording site via a fluid delivery unit such that the downward directed flow of delivery system solution from the overhead delivery system to the recording site directs fluid flow down toward and then away from the ion transport measuring hole, opposing “lateral” fluid flow of chamber solution to the site, so that during the fluid delivery period, fluid flow is outward from each recording site, and each recording site is covered by test solution that is not significantly diluted by chamber solution. The proximal and relatively rapid, relatively high-volume flow of compound solution from the fluid delivery units above the recording sites permits a window of time for ion transport measurement in which each recording site experiences an essentially undiluted compound concentration.

At the same time, flow of test solutions away from each of the two or more recording sites (to which test solutions are being delivered simultaneously) is accomplished by the continuous flow-through of chamber solution, which carries delivery solutions away from recording sites and out of the chamber. This provides effective fluid isolation of recording sites during ion transport measurement.

After ion transport recording, the flow of solution from fluid delivery units is halted, while fluid flow through the chamber continues. This allows the chamber, including the recording sites, to be washed. During the chamber wash, the fluid delivery units, which can be part of a fluidics block, can optionally move away from their recording site positions over the upper chamber, optionally be washed or flushed, and filled with a second set of test solutions. The fluidics block comprising the delivery units can then position back over the upper chamber, such that the individual fluid delivery units are aligned over individual recording sites, and a second set of compounds can be delivered to the recording sites. A second set of ion transport measurement assays can be performed as the second set of compounds is delivered to the recording sites.

The fluid isolation of recording sites during compound solution delivery and ion transport measurement can optionally be promoted by the use of flow retarding structures, upper chamber microwells, hydrophobic modifications to at least some portions a chip surface, or combinations of these, as discussed below.

The present invention includes methods of using ion transport measuring devices that include flow-through upper chambers and overhead fluid delivery systems to measure ion transport function and properties. In preferred embodiments, the methods are high throughput.

In a preferred embodiment, measuring solution is added to the one or more lower chambers of a device that comprises a chip having microwells that surround the holes of the chip and comprise the ion transport recording sites of the upper chamber, and measuring solution and cells are introduced into the upper chamber through conduits attached to one or more inlets. Pressure is applied through the lower chamber or chambers to seal particles against the ion transport measuring holes of the chip. Sealing preferably occurs in the presence of complete solution superfusion of the upper chamber. After the seals have formed, solution is removed from the upper channel, with the exception of the microwells, which in the case of a coated surface electrode, are in electrical connection with a reference electrode. At this time compounds are applied to the microwells by positioning the compound delivery system over the biochip and dispensing compound drops over the microwells. Ion transport measurement can then be performed on the cells sealed at the microwells.

In using this type of device, a single cell type can be added to this type of device via an inlet in the flow through upper chamber for screening different compound solutions that are delivered through openings in the upper chamber over the ion transport recording sites. Preferably, solution such as measuring solution flows continuously through the chamber during compound delivery and ion transport measurement.

Alternatively, different cell types or particles comprising different ion transports can be added at different ion transport recording sites. Immediately after cell addition, which can be through the fluid delivery units, pressure applied from the bottom of the chip can allow the cells (or other particles) to seal at ion transport measuring holes. In this way, a particular ion transport recording site can have a particular type or cell or particle sealed to it. Compounds can optionally be added through the chamber inlet or through openings in the chamber that are localized over the recording sites, and ion transport recordings can simultaneously measure the response of various cell types or ion channel types to one or more compounds. Optionally, the upper chamber channel can be flushed to remove the compound of interest, and a second compound can be added by pushing or pumping a second compound-containing solution into the channel. In this way, multiple compounds (or different concentrations of one or more compounds) can be assayed for their effects on one or more cell types or one or more ion transport types.

Flow Retarding Structures

A device of the present invention that comprises a chip with multiple ion transport measuring holes, a flow-through upper chamber, and an overhead fluid delivery system that can deliver solutions to individual recording sites can also comprise two or more flow-retarding structures that inhibit fluid flow to the ion transport recording sites of the device.

During the of such a device, ion transport measurement is performed as the upper chamber experiences continuous flow of chamber solution (such as a measuring solution) through the chamber, in which the chamber solution enters through a chamber inlet and exits through a chamber outlet, and as delivery solution (such as a compound solution) is delivered directly to two or more ion transport measuring sites via the overhead delivery system. One or more flow-retarding structures can be constructed that restrict the flow of chamber solution to ion transport recording sites, while still allowing the sites to be in fluid communication with the chamber.

Flow-retarding structures can be of any shape or size, as long as they permit fluid communication between recording sites and the chamber yet restrict laminar flow of chamber solution to the sites. Preferably, a flow-retarding structure is designed such that the flow of chamber solution to a recording site is essentially eliminated when a delivery solution is being delivered to the recording site via the overhead delivery system the chamber is experiencing continuous fluid flow of chamber solution. During this process the design permits fluid flow out of the recording site to the chamber. Thus structures for retarding fluid flow of chamber solution to a recording site can be designed and tested empirically for their effectiveness (for example using dye solutions) under various conditions of overhead delivery (including flow rate, aperture size of delivery unit, proximity of delivery unit to ion transport recording site, etc.) and fluid flow through the chamber (including flow rate, chamber dimensions, microwell dimensions, etc.).

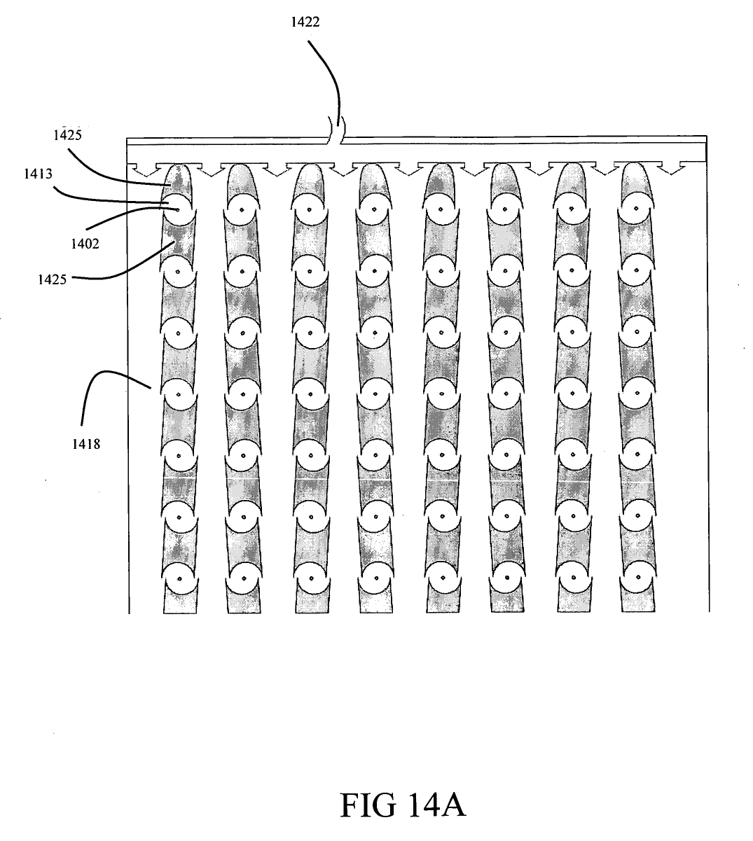

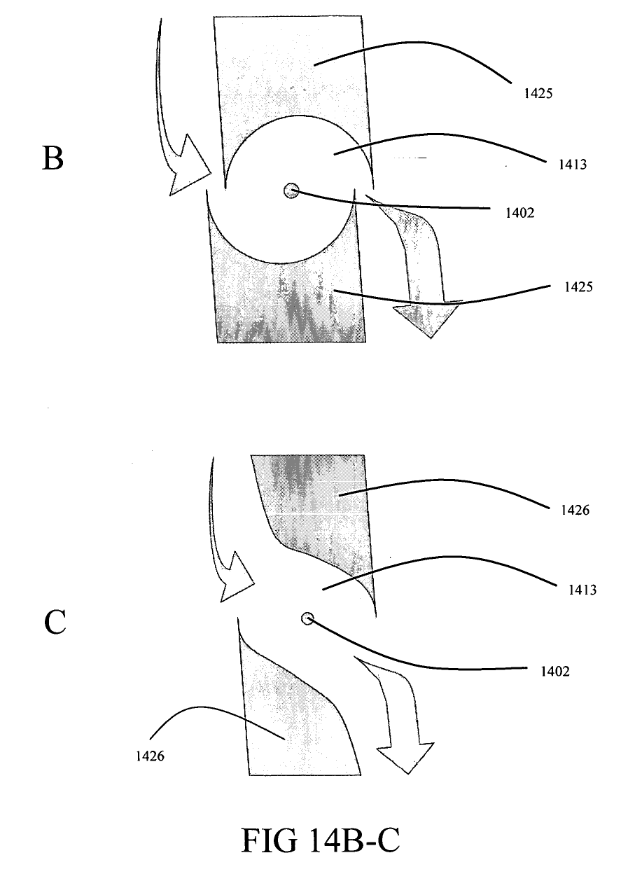

One example of flow-retarding structures is depicted in FIG. 14A. This figure depicts a portion of flow-through upper chamber (1418) depicting an inflow conduit (1422) flow-retarding structures (1425) positioned around each ion transport measuring recording site (1413), each of which surrounds an ion transport measuring hole (1402). FIGS. 14B and 14C are enlargements of two designs of flow-retarding structures (1425) surrounding a recording site (1413) that comprises an ion transport measuring hole (1402). The arrows show the pattern of flow of chamber fluid during washing of the chamber.

Fluidic Pipe Delivery

In some preferred aspects of the present invention, an ion transport measuring device comprises a biochip comprising two or more ion transport measuring holes, and at least one flow-through upper chamber positioned above the biochip that comprises two or more ion transport recording sites, each of which encompasses one of the two or more ion transport measuring holes of the biochip. The two or more ion transport recording holes access the one or more upper chambers at recording sites. The device further comprises a fluid delivery system comprising two or more fluid delivery units in the form of fluidic pipes, each of which can be aligned directly over one of the two or more ion transport recording sites. Solutions (including test compound solutions) can be added to ion transport recording sites that surround the ion transport recording holes through the fluid delivery units that can be positioned over the ion transport recording sites.

The flow-through upper chamber comprises at least one inlet and at least one outlet that can allow for fluid flow through the chamber. Chamber solutions (such as measuring solutions such as ES) and cells or compounds can be added via an upper chamber inlet. An electrode, such as a reference electrode, can optionally be provided within or, during use of the device, in electrical connection with the flow-through upper chamber.

In some preferred embodiments, a flow-through upper chamber of a device of the present invention comprises an upper surface that comprises two or more openings, in which each of the two or more openings is aligned over one of the two or more ion transport recording sites. The openings provide access of the fluidic pipes to ion transport recording sites of an upper chamber. In other embodiments, a chamber does not have an upper surface, and each of the two or more fluidic pipes can be aligned directly over the one of the two or more ion transport measuring recording sites and solutions can be added via fluidic pipes that are positioned over ion transport recording sites.

Some preferred devices of the present invention comprise a chip that comprises at least two ion transport measuring holes; at least one flow-through upper chamber that is accessed by the at least two ion transport measuring holes; and at least two fluidic pipes that can be positioned over the at least one upper chamber, in which each of the at least two pipes aligns directly over and in close proximity to an ion transport measuring hole. The pipes are connected to conduits of a fluidics system that feeds solutions, such as test compound solutions, through the pipes to ion transport recording sites during ion transport measurement assays.

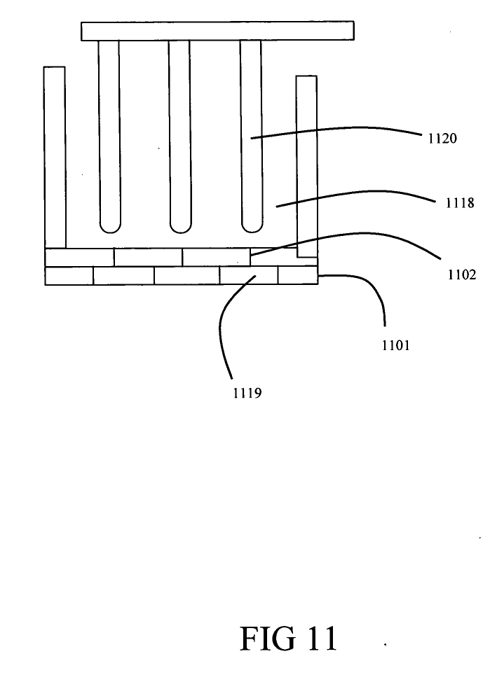

FIG. 11 depicts one embodiment of a fluidic pipe overhead delivery system. In this embodiment, a device has a common upper flow-through chamber (1118) (inlet and outlet not depicted) positioned over a chip (1101) that comprises multiple ion transport measuring holes (1102). In this embodiment, the device also comprises multiple lower chambers (1119), each of which is in register with a single ion transport measuring hole (1102). Fluidic pipes (1120) are depicted positioned over the ion transport measuring holes (1102). The pipes are used for delivery of solutions, such as test compound solutions, to ion transport recording sites during ion transport measurement assays.

A pipe used as a fluid delivery unit comprises a conduit outlet at the delivery end (the end that is positioned over the ion transport recording site during use of the device) that can provide continuous flow of a solution to the ion transport recording site. Preferably, the opposite end of the pipe connects to at least one solution reservoir. In some preferred aspects of the invention, the pipe connects to two or more sources of solutions, at least one of which can be an assay solution (for example, a test compound solution) and at least one other or which can be a wash solution or a standard measuring solution (such as ES). Preferably, the pipe engages a conduit that engages a valve that allows switching between solutions that flow through the pipe. For example, the valve, which preferably can be automatically controlled, can allow compound solution to flow through the pipe for a period of time, followed by wash solution, optionally followed by a second compound solution.

The present invention also includes methods of using ion transport measuring devices that comprise pipe arrays for delivering compounds at ion transport measuring sites of upper chambers. In broad outline, such methods include: providing measuring solution in the lower chambers of the device; providing particles in measuring solution in an upper chamber of the device; sealing particles at ion transport measuring holes; providing continuous flow of measuring solution through the upper chamber; positioning an array of pipes over the upper chamber; delivering compounds continuously at recording sites through the pipes, and measuring ion transport function or properties. The upper chamber of the ion transport measuring device can optionally be flushed after ion transport measurement, and optionally wash solution followed by a new compound solution can be added to upper chamber recording sites using the pipe array. The process can be repeated multiple times.

The pipes can also be used to deliver tiny amount of compounds in drops to cells already sealed to the recording sites in low volume of solutions, such as, for example, those in the microwells of a chip that comprises a hydrophobic surface between the microwells. In this case, chamber solution is removed from the upper chamber, with the exception of the microwells, prior to overhead delivery of test solutions. Fusion of the test solution drop and the small volume of measuring solution at the recording sites allows for fast and efficient compound delivery. The fused drops in the microwell recording sites will not fuse together to cross-contaminate since the drops are bounded by hydrophobic coatings. Wash out can be achieved by flushing the entire upper chamber with wash solution and subsequent removal of wash solution. The recording sites are then ready to receive the next delivery of compounds.

Multichannel Pipet Delivery of Compounds

In some preferred aspects of the present invention, an ion transport measuring device comprises a biochip comprising two or more ion transport measuring holes, and at least one flow-through upper chamber positioned above the biochip that comprises two or more ion transport recording sites, each of which encompasses one of the two or more ion transport measuring holes of the biochip. The two or more ion transport recording holes access the one or more upper chambers at recording sites. The device further comprises a fluid delivery system comprising two or more fluid delivery units in the form of multichannel pipets, each of which can be aligned directly over one of the two or more ion transport recording sites. Solutions (including test compound solutions) can be added to ion transport recording sites that surround the ion transport recording holes through the multichannel pipets that can be positioned over the ion transport recording sites.

The flow-through upper chamber comprises at least one inlet and at least one outlet that can allow for fluid flow through the chamber. Chamber solutions (such as measuring solutions such as ES) and cells or compounds can be added via an upper chamber inlet. An electrode, such as a reference electrode, can optionally be provided within or, during use of the device, in electrical connection with the flow-through upper chamber.

In some preferred embodiments, a flow-through upper chamber of a device of the present invention comprises an upper surface that comprises two or more openings, in which each of the two or more openings is aligned over one of the two or more ion transport recording sites. The openings provide access of the pipets to ion transport recording sites of an upper chamber. In other embodiments, a chamber does not have an upper surface, and each of the two or more pipets can be aligned directly over the one of the two or more ion transport measuring recording sites and solutions can be added via pipets that are positioned over ion transport recording sites.

Some preferred devices of the present invention comprise a chip that comprises at least two ion transport measuring holes; at least one flow-through upper chamber that is accessed by the at least two ion transport measuring holes; and at least two multichannel pipets that can be positioned over the at least one upper chamber, in which each of the at least two pipes aligns directly over and in close proximity to an ion transport measuring hole.

A multichannel pipet used as a fluid delivery unit can dispense solution directly to an ion transport recording site. Preferably, the pipet is part of a fluidic block that can move from an uptake position for receiving compound for dispensing to the dispensing position over the upper chamber. In some preferred aspects of the invention, the pipet can be used to sequentially dispense two or more different compound solutions that are dispensed in successive assays. Between assays, the chamber is washed using the flow-through conduits.

The present invention also includes methods of using ion transport measuring devices that comprise dispensing pipet arrays for delivering compounds at ion transport measuring sites of upper chambers. In broad outline, such methods include: providing measuring solution in the lower chambers of the device; providing particles in measuring solution in an upper chamber of the device; sealing particles at ion transport measuring holes; providing continuous flow of measuring solution through the upper chamber; positioning an array of dispensing pipets over the upper chamber; dispensing compounds at recording sites through the pipets, and measuring ion transport function or properties. The upper chamber of the ion transport measuring device can optionally be flushed after ion transport measurement, and optionally new compound solutions can be added to upper chamber recording sites using the pipet array. The process can be repeated multiple times.

In some preferred aspects of the present invention, the chip comprises microwells that comprise ion transport recording sites, and the top surface of the chip, with the exception of the microwell surfaces, is preferably hydrophobic to aid in maintaining fluid separation between microwells when fluid is removed from the upper chamber.

In this embodiment, the compound delivery system can deliver compound solution from pipets over the microwells in droplets that localize to the microwells and do not spread to other wells due in part to the hydrophobicity of the chip upper surface. Preferably, the compound drops are very large compared to the microwell volume, so that there is little compound dilution when it is delivered. In this case, after particle sealing, chamber solution is removed from the upper chamber, with the exception of the microwells, prior to overhead delivery of test solutions.

Fusion of the compound drop and the small volume of solutions at the recording sites allows for fast and efficient compound delivery. The fused drops will not fuse together to cross-contaminate recording sites since the drops are bounded by the hydrophobic chip surface outside the microwells.

Wash out can be achieved by flushing the entire upper chamber with wash solution and subsequent removal of wash solution. After washout, recording sites are ready to receive the next delivery of compounds.

Sonic Actuators

In a related embodiment, the fluid delivery units are sonic actuators that can be part of a block or plate comprising solutions in wells that is localized over the ion transport recording sites of a device. Activation of a sonic actuator on the plate causes a droplet of solution from a well associated with actuator to be ejected out of the well to the ion transport measuring site. In a preferred aspect of this embodiment, the compound delivery system can deliver compound solution from delivery wells positioned over the upper chamber microwells in droplets that localize to the microwells and do not spread to other wells. The fluid isolation of the microwells can be promoted by having a hydrophobic chip surface outside the microwells. In this case, after particle sealing, chamber solution is removed from the upper chamber, with the exception of the microwells, prior to overhead delivery of test solutions. Fusion of the compound drop and the small volume of solutions at the recording sites allows for fast and efficient compound delivery. The fused drops will not fuse together to cross-contaminate recording sites since the drops are bounded by the hydrophobic chip surface between microwells.

Wash out can be achieved by flushing the entire upper chamber with wash solution and subsequent removal of wash solution. After washout, recording sites are ready to receive the next delivery of compounds.

In an alternative, the sonic actuators can be part of a block or plate that is positioned under the lower microwells of an ion transport measuring device. In this case, particles such as cells are in the lower chamber and are sealed to the lower surface of the chip by pneumatic devices connected to the upper wells. After particle sealing, chamber solution is removed from the lower chamber, with the exception of the microwells, prior to delivery of test solutions from below. Activation of a sonic actuator on the plate causes a droplet of solution from a well associated with actuator to be ejected upward out of the well to the ion transport measuring site of a lower well. Fusion of the ejected compound drop and the small volume of solution at a recording site allows for fast and efficient compound delivery. In both of these variations, a droplet of fluid ejected by a sonic actuator fuses with the small amount of solution surrounding the sealed cell at the ion transport measuring site. Ion transport measurement can be performed after compound solution delivery, and washout of the flow-through upper or lower chamber can be performed after recordings have been performed.

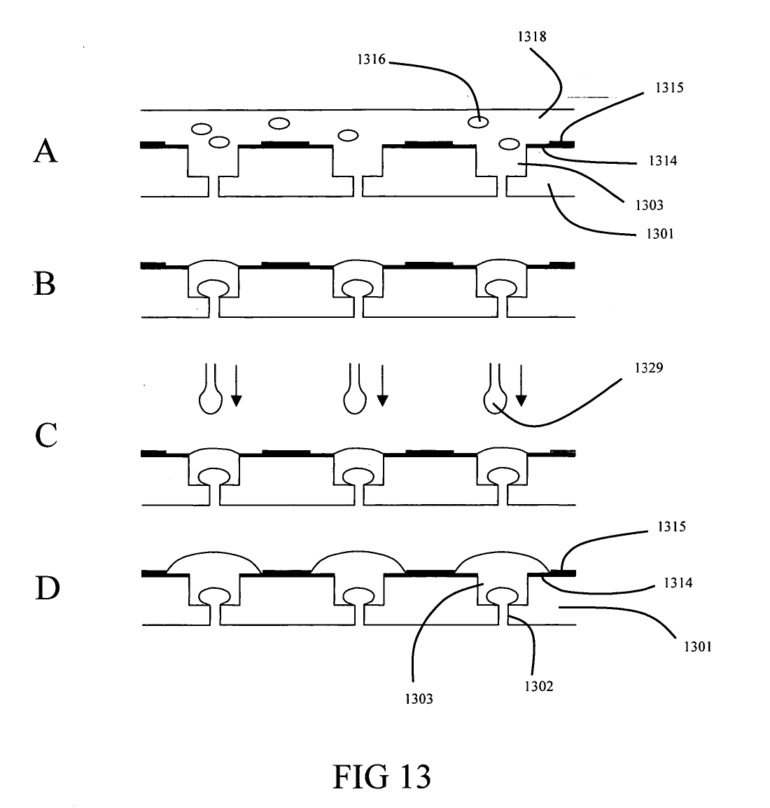

FIG. 13 depicts compound delivery from a fluid delivery unit positioned over microwells of a flow-through upper chamber of a device of the present invention. In FIG. 13A, the flow-through upper chamber is perfused with chamber (measuring) solution that comprises cells (1316). The upper chamber has microwells (1303) surrounding ion transport measuring holes (1302) in the chip (1301). The chip also compises a hydrophobic coating (1315) that surrounds but does not contact the microwells. In FIG. 13B, cells (1316) are sealed to ion transport measuring holes (1302) in the microwells (1303). This can be accomplished by the application of pressure via conduits that engage the lower chambers and lead to a pneumatic device. In FIG. 13C, a drop of compound solution (1329) is dispensed, from, for example, a sonic actuator plate or multichannnel pipet. In FIG. 13D, the drop of compound solution has fused with the solution in the microwell (1303). The microwells (1303) are in fluid isolation from one another.

Nozzles

In some preferred embodiments of the novel fluidic systems of the present invention, an ion transport measuring device that comprises a biochip comprising two or more ion transport measuring holes, and at least one flow-through upper chamber positioned above the biochip, and a fluid delivery system comprising two or more fluid delivery units can further comprise two or more nozzle structures positioned over the ion transport measuring sites that can engage the fluid delivery units of the fluid delivery system.

The outflow nozzle structures can be reversibly or irreversibly aligned over the chip such that a single nozzle is positioned over each ion transport recording site, or can be can be a part of the piece that comprises the upper chamber walls. In either case, for the dispensing of solutions to ion transport measuring sites, the nozzles are positioned over the ion transport measurement recording sites of the device such that the fluid delivery units of the overhead fluid delivery system can dispense fluid into the nozzles that then flows to the ion transport measurement recording sites.

Preferably, when the fluid delivery system is aligned over the chip, the outflow nozzles are positioned close to the surface of the measuring solution of the upper chamber, but not in contact with it. Preferably, the nozzle is at the end of a funnel structure, the nozzle diameter is greater than ten times the diameter of the cells, and the funnel size is large enough to allow the compound solution within it to flow out of the nozzle over sufficient time that more compound solution can be delivered to the funnels (such as by dispensing pipette tips) without creating bubbles within the funnel or nozzle area.

Preferably, the device comprises or engages at least two lower chambers in register with the two or more holes of the chip. During ion transport measurement, each of the individual lower chambers preferably comprises or is in electrical contact with a recording electrode.

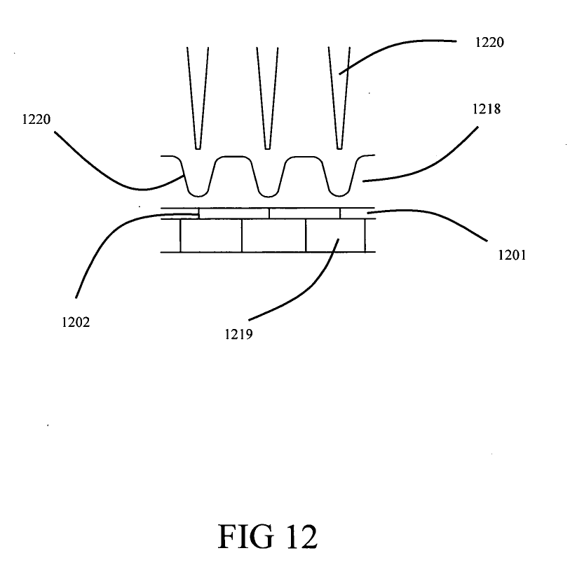

One design of this aspect of the present invention is depicted in FIG. 12. In this embodiment, a device has a common upper flow-through chamber (1218) (inlet and outlet not depicted) positioned over a chip (1201) that comprises multiple ion transport measuring holes (1202). In this embodiment, the device also comprises multiple lower chambers (1219), each of which is in register with a single ion transport measuring hole (1202). Fluidic pipes (1120) are depicted positioned over the ion transport measuring holes (1202). The device also comprises nozzles (1221) positioned over the ion transport measuring holes (1202).

In using the device, lower chambers are filled with measuring solutions, and the the upper chamber is filled with measuring solution and cells (or other particles) are added. Cells (or other particles) are sealed to the ion transport measuring holes by applying suction to the lower chambers. By controlling fluid flow through the upper chamber, an even but shallow bath is produced that has continuous flow. The fluidic delivery units, (for example, pipets or pipes) for compound addition are positioned over the holes on the chip such that the outflow nozzles are close to the surface of the measuring solution within the upper chamber, but not in contact with it. After control currents are recorded, compound solutions are added to the nozzles from above, such as by pipets or fluidic pipes.

As compound solutions flow through the nozzles to ion transport recording sites, the fluid delivery system comprising an array of pipets or fluidic pipes can move away from the for uptake of other compound solutions (in the case of pipets) or for flushing the delivery units of a first compound solution prior to filling them with a second compound solution. The fluidics block comprising the array of fluid delivery units can then move back to the nozzles over the ion transport recording sites for delivery of a second set of compound solutions to the ion transport recording sites.

The present invention includes ion transport measuring devices that include a biochip comprising ion transport two or more ion transport measuring holes and a compound delivery system that can deliver compound or solution to each ion transport measurement site individually, and nozzles positioned over each ion transport recording site. In preferred embodiments, the devices are high-throughput devices that comprise at least 48, at least 96, or at least 384 ion transport measuring sites and a corresponding number of nozzles for dispensing compounds over the ion transport measuring sites.

Device Having Compound Delivery Plate

Yet another aspect of the present invention is an ion transport measuring device that comprises a substrate comprising at least two ion transport measuring holes, at least two upper chambers in register with the two or more ion transport measuring holes; at least two lower microwells, each of which is positioned around an ion transport measuring hole, and each of which is connected to a common lower chamber channel; and a compound delivery plate, in which the compound delivery plate has drug delivery sites in register with the lower microwells, where the compound delivery plate can reversibly come into contact with the lower microwells. In this design, depicted in FIG. 13, the two or more upper chambers are connected to a pneumatic system for sealing cells to the ion transport measuring holes on the lower side of the substrate and each of the upper chambers comprises or is in electrical contact with an individual (recording) electrode.

Electrical and pneumatic connection to the upper wells of the ion transport measuring chip can optionally be provided by a separate adaptor plate. Preferably, each independent upper well connects to a separate recording electrode.

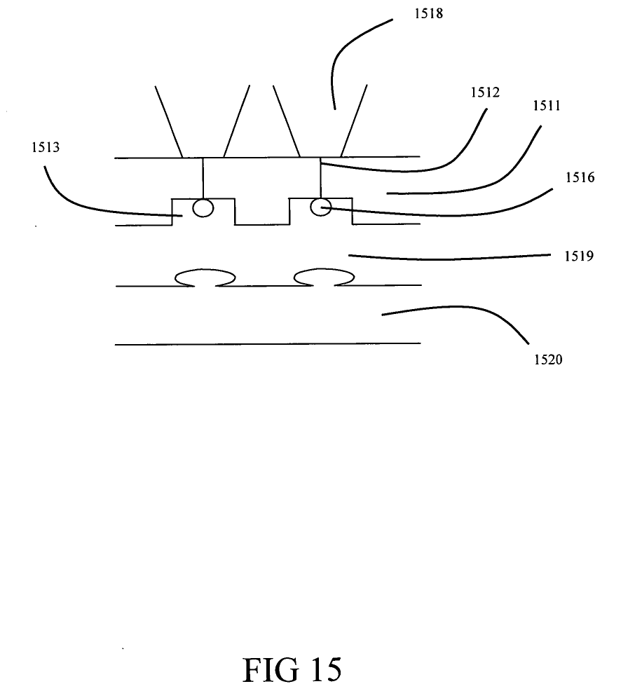

FIG. 15 depicts a device in which the lower channel (1519) which accesses the lower microwells (1513)) is a flow-through chamber having fluid flow of measuring solution through the channel. The bottom surface of the chip (1511), with the exception of the microwell surfaces, is preferably hydrophobic to aid in maintaining fluid separation between microwells when fluid is removed from the lower chamber. A reference electrode can preferably be provided on the lower surface of the chip, connected to the compound delivery plate (1520), or in electrical contact with the lower channel.

In some preferred designs, at the time of operation of the device, the drug delivery sites have compounds spotted, or printed on them in drops or dried form.

In operation, measuring solution is added to the upper chambers, and measuring solution and cells are introduced into the lower chamber channel through conduits. Pressure is applied to the upper wells (either individually or commonly connected to pressure controls) to pull cells up from the lower channel into the lower microwells and seal them against the ion transport measuring holes of the chip. Sealing occurs in the presence of complete solution superfusion of the bottom chamber. After the seals have formed, solution is removed from the channel, with the exception of the microwells, which in the case of coated surface electrodes, are in electrical connection with the electrodes. At this time compounds are applied to the microwells as the delivery plate is brought into contact with the lower surfaces of the microwells. Ion transport measurement can then be performed.

The same device can be used in inverted orientation, with cells sealing to the top of the chip, and the compound delivery plate is positioned above the chip to apply compounds from the top side of the chip.

### FURTHER EMBODIMENTS

The present invention includes chips, devices, and methods for ion transport measurement that can be used to efficiently assay test particles such as cells. The devices allow ion transport assays that can be used in a variety of ion transport measurement applications, including but not limited to determining the effects of compounds (such as compounds of interest or test compounds) on ion transport activity. The assays can also be used to assess cell condition, “sealability”, responsiveness to compounds or treatments before performing a battery of tests using the cells, or to rapidly determine the effects of growth conditions, developmental stages, hormone responsiveness on the ion transport activity of cells. In some embodiments, the ion transport measuring devices can be used for other assays in addition to ion transport measurement assays. In some embodiments, the ion transport measuring devices can designed such that cells in a chamber of the device can be microscopically viewed before, during, and/or immediately after an ion transport measurement assay.

Method for Performing Excised Patch Voltage Clamp Recordings

Excised voltage clamp recordings such as inside-out or outside in configurations as known in the art of voltage clamp studies can be performed by any planar or non-planar electrode configurations known in the art, or described in this application or previous applications. This is done by adding magnetic beads labeled with antibody(s) against common cell surface markers after the cell is sealed to the ITM sites; incubation to allow for bead binding to the cell surface; and applying magnets to the beaded sealed cells from the open access. Magnetic forces will remove the beads, together with associated cell membrane, which allows the formation of “excised patch” configuration at the ion transport measuring sites for single channel or macropatch recordings.

Method of Shipping Ion Transport Measuring Chips The present also provides methods for shipping ion transport measuring chip and devices in which the upper and lower chambers of the devices or chips are pre-filled with an ion transport measuring solution. For example, where the devices are intended for use in performing ion transport measurement assays on whole cells, the devices can be packed with upper and lower chambers filled with intracellular solution (1S). This can reduce the time required to perform an assay, and also can reduce the complexity of the machinery that interfaces with the device and provides fluidic controls and conduits, since the machinery does not need to add measuring solution to, for example the lower chambers of a device, but instead can simply flush the upper chamber with an appropriate measuring solution such as extracellular solution (ES) prior to adding cells. This increases the efficiency and reduces the time needed for assays, such as high throughput screens. (In cases such as that described in Aspect 28, where cells are distributed in lower chambers, the machine flushes the lower chamber with, for example, ES, prior to adding cells.) The devices or chips can be shipped in blister packs that lock in the measuring solution, and the entire assembly can optionally be kept refrigerated until use. The measuring solution used can be specialized for different types of ion transport assays, different cell types, and the like. The measuring solution can also be simplified for more general use with more than one cell or assay type.

To use devices shipped in measuring solution, after flushing extracellular solution through and adding cells to the one or more upper chambers, a vacuum can be applied to the one or more intracellular chambers that already contain IS to seal cells to ion transport measuring holes.

The aspects of the invention disclosed herein can be combined to make new embodiments that are also within the scope of the invention. The aspects of the invention disclosed herein, such as, but not limited to chip designs, chamber designs, electrode arrangements and connections, through-hole designs and manufacture, fluidics arrangements, etc. can be combined with other features described herein, known in the art, or features that are developed in the future.

All references cited herein, including patents, patent application, and publications, are incorporated herein by reference in their entireties.

All headings are for the convenience of the reader and should not be used to limit the meaning of the text that follows the heading, unless so specified.

1-174. (canceled)

175. A hydrophilic biochip for ion transport measurement comprising a substrate that comprises:
one or more holes;
one or more hydrophilic recording site areas, wherein each of said one or more hydrophilic recording site areas on the particle-sealing side of said biochip; and
at least one hydrophobic area comprising the surface of said substrate surrounding said at least one hydrophilic recording site area;
wherein said at least one hydrophobic area can maintain an aqueous solution localized to said nonhydrophobic recording site area in fluid isolation.

176. The biochip of claim 175, wherein said substrate comprises two or more holes and two or more hydrophilic recording site areas, wherein the area immediately surrounding each of said two or more holes is hydrophilic, and wherein an aqueous solution provided in the hydrophilic recording site area surrounding any of said two or more holes is isolated from an aqueous solution provided in the hydrophilic recording site area surrounding any other of said two or more holes.

177. The biochip of claim 176, wherein said one or more hydrophilic recording site areas of said substrate is negatively charged.

178. The biochip of claim 177, wherein negative charges of said one or more hydrophilic recording site areas are counterbalanced by noncovalently bound positive charges.

179. The biochip of claim 175, wherein said one or more hydrophilic recording site areas can hold a drop of aqueous liquid of a volume of from about 1 microliter to about 2 milliliters.

180. The biochip of claim 175, wherein said one or more hydrophilic recording site areas have a diameter of from about 25 micron to about 10 millimeters.

181. The biochip of claim 175, wherein said biochip comprises a hydrophilic substrate, wherein said one or more hydrophobic barrier areas comprise a hydrophobic modification or coating on the surface of said hydrophilic substrate.

182. The biochip of claim 176, wherein said hydrophilic substrate comprises glass, silicon, silicon dioxide, quartz, or one or more polymers.

183. The biochip of claim 182, wherein said hydrophilic substrate is from about 1 micron to about 2 millimeters thick.

184. The biochip of claim 182, wherein said hydrophobic modification or coating comprises coating of at least one plastic or at least one polymer.

185. The biochip of claim 184, wherein said coating comprises a layer of said hydrophobic material of at least 1 molecular layer in thickness.

186. The biochip of claim 175, further comprising one or more microwells, wherein each of said one or more microwells surrounds one of said one or more holes.

187. The biochip of claim 175, wherein said one or more holes have a diameter of between about 0.2 micron and about 10 microns.

188. The hydrophobic ion transport measurement chip of claim 187, comprising at least eight holes.

189. A method of making a hydrophilic chip, comprising:
providing a substrate that comprises a hydrophilic material;
coating said substrate with at least one hydrophobic material;
making at least one hole through said substrate; and
removing said hydrophobic substrate from an area immediately surrounding said at least one hole.

190. The method of claim 189, further comprising chemically treating said area immediately surrounding said at least one hole to improve its electrical sealing properties.

191. The method of claim 190, wherein said chemically treating comprises treating said area immediately surrounding said at least one hole with at least one salt or at least one base.

192. The method of claim 189, wherein said removing comprises drilling or etching at least one microwell around said at least one hole.

193. A method of making a hydrophilic chip, comprising:
providing a substrate that comprises a hydrophilic material;
making at least one hole through said substrate; and
coating at least a portion of said substrate with at least one hydrophobic material, wherein from an area immediately surrounding said at least one hole is masked to prevent it from receiving said coating.

194. A method of making a hydrophilic chip, comprising:
providing a substrate that comprises a hydrophobic material;
making at least one hole through said substrate; and
coating an area immediately surrounding said at least one hole with at least one hydrophilic material.

195. The method of claim 194, further comprising chemically treating said area immediately surrounding said at least one hole to improve its electrical sealing properties.

196. The method of claim 195, wherein said chemically treating comprises treating said area immediately surrounding said at least one hole with at least one salt or at least one base.

197. A method of making an ion transport measurement microchannel plate (MCP), comprising:
a) providing an MCP comprising at least two microchannels;
b) chemically treating at least one surface of said microchannel plate or a portion thereof to increase the electrical sealing properties of said at least two microchannels.

198. A method of making a flexible chip for ion transport measurement, comprising:
a) providing a substrate comprising at least one flexible material; and
b) making at least one hole through said substrate to make a flexible ion transport measuring chip.

199. The method of claim 198, wherein said making at least one hole comprises laser drilling, chemical etching, molding, milling, or micromachining at least one hole.

200. The method of claim 198, further comprising making at least a portion of the particle-sealing surface of said flexible ion transport measuring chip hydrophilic.

201. The method of claim 200, further comprising coating at least a portion of said substrate with silicon dioxide or glass.

202. The method of claim 200, further comprising chemically treating said flexible chip to increase its electrical sealing properties.

203. The method of claim 202, wherein said treating comprises treating with at least one salt or at least one base.

204. The method of claim 198, further comprising drilling at least one counterbore for said at least one hole.

205. The flexible ion transport measuring chip made by the method of claim 198, wherein said substrate comprises rubber, at least one plastic, or at least one polymer.

206. The flexible ion transport measuring chip made by the method of claim 198, wherein said substrate is between about 5 microns and about 5000 microns thick.

207. A flexible chip extension device comprising:
a) the flexible chip of claim 205;
b) a first spool around which said flexible chip is wound to produce a chip roll having a leading edge; and
c) a second spool or guide positioned at a distance from said first spool that engages said leading edge.

208. An ion transport measuring device comprising:
a) the flexible chip extension device of claim 207,
b) at least one upper chamber piece that forms at least the walls of at least two upper chambers; and
c) at least one lower chamber piece that forms at least the walls of at least one lower chamber.

209. The ion transport measuring device of claim 208, wherein said at least one lower chamber is one lower chamber.

210. The ion transport measuring device of claim 209, further comprising:
d) at least two upper chamber electrodes, wherein each of said at least two electrodes contacts or can be positioned to be in electrical contact with one of said at least two upper chambers; and
e) a lower chamber electrode that contacts or can be positioned to be in electrical contact with said one lower chamber.

211. A method of using the ion transport measuring device of claim 210, comprising:
a) connecting said two or more upper chamber electrodes and said lower chamber electrode to two or more signal amplifiers;
b) dispensing a sample comprising at least one particle into at least one upper chamber of the device of claim 210;
c) sealing at least one particle to at least one of said two or more holes of said device of claim 210; and
d) measuring ion transport activity of said at least one particle.

212. The flexible chip of claim 205, wherein said flexible chip forms a cylinder.

213. A method of making an ion transport measuring device, comprising:
a) providing at least two theta tubing segments, wherein each theta tubing segment comprises an upper compartment and a lower compartment separated by a glass septum;
b) cutting openings in the tops of said at least two theta tubing segments to provide access to the upper compartments of said at least two theta tubing segments;
c) using said openings to laser drill or etch at least one hole through the glass septum of each of said at least two theta tubing segments;
d) sealing said openings in the tops of said theta tubing segments after laser drilling or etching said at least one hole;
e) attaching said at least two theta tubing segments on top of one another or side-by-side; and
f) attaching conduits to said upper compartments and said lower compartments.

214. A device for ion transport measurement, comprising:
a) a chip comprising one or more ion transport measuring holes; and
b) one or more upper chambers situated above said chip such that each of said one or more upper chambers is accessed by at least one of said one or more ion transport measuring holes;
further wherein said at least one upper chamber comprises at least two openings,
wherein at least one of said at least two openings is at least one inlet on one side of said at least one ion transport measuring hole, and at least one other of said at least two openings is at least one outlet on the opposite side of said at least one ion transport measuring hole.

215. The device of claim 214, wherein said at least one outlet engages an outflow conduit.

216. The device of claim 215, wherein said at least one inlet engages an inflow conduit.

217. The device of claim 216, wherein said at least one inlet connects to a reservoir.

218. The device of claim 214, wherein said one or more upper chambers have a top surface that is transparent.

219. The device of claim 214, wherein said one or more upper chambers is one upper chamber.

220. The device of claim 219, wherein said one or more ion transport measuring holes are two or more ion transport measuring holes, further wherein each of said one or more upper chambers is accessed by at least two of said two or more ion transport measuring holes and further wherein said at least one inlet engages an inflow conduit and said at least one outlet engages an outflow conduit.

221. The ion transport measuring device of claim 220, further comprising at least two fluid delivery units that can be positioned over said at least one upper chamber, wherein each of said at least two fluid delivery units aligns directly over and in close proximity to one of said two or more ion transport measuring holes at two or more recording sites.

222. The ion transport measuring device of claim 221, wherein said at least two fluid delivery conduits units comprise multichannel pipets or fluidic pipes.

223. The device of claim 222, wherein said at least two fluid delivery units comprise funnel structures, wherein said funnel structures can restrict the flow-through of fluids at said two or more recording sites.
